(19) United States
(12) Patent Application Publication
(10) Pub. No.: US 2024/0399129 A1
(43) Pub. Date: Dec. 5, 2024

(54) KINETIC VACUUM TREATMENT DEVICES, SYSTEMS, AND METHODS

(71) Applicant: Inovio Pharmaceuticals, Inc., Plymouth Meeting, PA (US)

(72) Inventors: Paul Fisher, San Marcos, CA (US); Ryne Contreras, Oceanside, CA (US); Alison A. Generotti, Ocean City, NJ (US); Jay McCoy, Temecula, CA (US); Brenden Zounes, San Diego, CA (US)

(21) Appl. No.: 18/648,280

(22) Filed: Apr. 26, 2024

Related U.S. Application Data
(60) Provisional application No. 63/498,690, filed on Apr. 27, 2023.

(51) Int. Cl. A61M 37/00

(52) U.S. Cl. CPC A61M 37/00; A61M 2037/0007

(57) ABSTRACT

A method of enhancing delivery of an agent into tissue includes placing a housing that defines a chamber adjacent to a surface of the tissue, thereby locating the chamber adjacent to the injection site at which the agent was injected into the tissue, applying vacuum pressure to the chamber, thereby drawing a portion of the tissue through an opening of the chamber and into the chamber, and moving the housing relative to the tissue while the vacuum pressure is applied for enhancing agent delivery in the tissue.

### CROSS-REFERENCE TO RELATED APPLICATIONS

The present application claims the benefit of U.S. Provisional Application No. 63/498,690, filed Apr. 27, 2023, the entire contents of which are incorporated herein by this reference.

### TECHNICAL FIELD

The present invention relates to devices, assemblies, and systems for gripping and/or deforming tissue with vacuum pressure and delivering fluid into the tissue.

### BACKGROUND

Numerous and varied techniques have been developed for enhancing delivery of agents in vivo into tissue. Some of these techniques involve modes of injecting the agent into the tissue. Such modes including certain types of needle injection (e.g., Mantoux injection, side-port injection) and other injection types, such as jet injection. Other techniques for enhancing agent delivery in vivo involve modes of spreading or dispersing an injected agent within the tissue and/or across various layers of tissue or across the same layer of tissue. Iontophoresis, microneedle arrays, suction-cup or vacuum-cup treatments are examples of such modes. Yet other techniques for enhancing agent delivery in vivo involve modes that increase agent uptake directly into target cells within the tissue. Electroporation and sonoporation are examples of such a mode. Additional techniques for enhancing agent delivery in vivo involve modes that include pairing the agent with an adjuvant known to enhance the immune response(s) elicited by the agent. Some techniques accomplish multiple of the foregoing modes for enhancing agent delivery in vivo. For example, vacuum-assisted electroporation (VEP) treatments have been shown to enhance agent delivery in vivo by mode of increasing injectate dispersion within tissue and by mode of increasing agent uptake directly into target cells within the tissue.

### SUMMARY

According to an embodiment of the present disclosure, a method of enhancing delivery of an agent into tissue includes placing a housing that defines a chamber adjacent to a surface of the tissue, thereby locating the chamber adjacent to the injection site at which the agent was injected into the tissue, applying vacuum pressure to the chamber, thereby drawing a portion of the tissue through an opening of the chamber and into the chamber, and moving the housing relative to the tissue while the vacuum pressure is applied for enhancing agent delivery in the tissue.

According to another embodiment of the present disclosure, a method of enhancing delivery of an agent into tissue includes injecting an agent into tissue of the subject, thereby defining an injection site at a surface of the tissue, placing a housing that defines a chamber at or adjacent the injection site, and applying vacuum pressure to the chamber, thereby drawing a portion of the tissue through an opening of the chamber and into the chamber. While the vacuum pressure is applied, the housing is moved relative to the tissue for enhancing agent delivery in the tissue.

According to an additional embodiment of the present disclosure, a system for vacuum-enhanced agent delivery into tissue in vivo includes a housing defining a chamber and an opening into the chamber, and at least one port extending through the housing. The at least one port is remote from the at least one opening and is connectable to a vacuum source, such that the at least one port is configured to communicate vacuum pressure from the vacuum source to the chamber. The housing is configured to communicate a vacuum field to a portion of the tissue and thereby draw the portion of tissue through the opening and at least momentarily hold the portion of the tissue in the chamber. The housing is further configured to move relative to the tissue while the vacuum field is communicated to the tissue, wherein the relative motion deforms at least some of the tissue. The system includes one or more features that are disposable between a distal end of the housing and a surface of the tissue for reducing sliding friction between the housing and the tissue.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing summary, as well as the following detailed description of illustrative embodiments of the present application, will be better understood when read in conjunction with the appended drawings. For the purposes of illustrating the features of the present application, there are shown in the drawings illustrative embodiments. It should be understood, however, that the application is not limited to the precise arrangements and instrumentalities shown. In the drawings:

FIG. 1A is a diagrammatical view of a kinetic vacuum treatment system that employs a vacuum cup, according to an embodiment of the present disclosure;

FIG. 1B is a perspective view of the vacuum cup illustrated in FIG. 1A, showing a vacuum chamber of the cup, according to an embodiment of the present disclosure;

FIG. 1C is a sectional side view of the vacuum cup illustrated in FIG. 1B, taken along a sectional plane that extends along a central axis of the cup;

FIG. 1D is another sectional side view of the vacuum cup illustrated in FIG. 1B, showing a mound of tissue drawn into the vacuum chamber responsive to application of vacuum pressure, and also showing fluid injectate dispersed within the skin layer of the tissue;

FIG. 2A is a perspective view of a vacuum cup having a centerpost positioned within the vacuum chamber, according to another embodiment of the present disclosure;

FIG. 2B is a sectional side view of the vacuum cup illustrated in FIG. 2A, taken along a sectional plane that extends along a central axis of the cup;

FIG. 2C is another sectional side view of the vacuum cup, similar to that shown in FIG. 2A, showing a mound of tissue drawn into the vacuum chamber and into contact with the centerpost, responsive to application of vacuum pressure, and also showing fluid injectate dispersed within the skin layer of the tissue;

FIG. 3A is a sectional side view of a vacuum cup, similar to the cup shown in FIGS. 1A-1D, having an injection channel for an injection needle, according to another embodiment of the present disclosure;

FIG. 3B is a sectional side view of a vacuum cup, similar to the cup shown in FIGS. 2A-2C, having a jet injector that extends into the vacuum chamber, according to another embodiment of the present disclosure;

FIG. 4A is a perspective view of a vacuum cup having electrodes for electroporating tissue drawn in to the vacuum chamber, according to another embodiment of the present disclosure;

FIG. 4B is a sectional side view of the vacuum cup illustrated in FIG. 4A;

FIG. 5A is a perspective view of a handle assembly that includes a vacuum cup, according to another embodiment of the present disclosure;

FIG. 5B is a partially exploded view of the handle assembly illustrated in FIG. 5A, showing the vacuum cup detached from a mounting formation of the handle assembly, the vacuum cup rotated into a bottom view for illustrative purposes, according to an embodiment of the present disclosure;

FIGS. 6A-6G are sectional side views of tissue showing representative stages of an exemplary kinetic vacuum treatment that employs the vacuum cup illustrated in FIGS. 1A-1D, according to an embodiment of the present disclosure;

FIG. 6H is a sectional side view of tissue showing representative stages of another exemplary kinetic vacuum treatment;

FIGS. 7A-7F are plan views showing exemplary kinetic movements of a vacuum cup for providing kinetic vacuum treatments, according to embodiments of the present disclosure; FIG. 7A shows side-to-side translational cup movements; FIG. 7B shows up-and-down or forward-and-backward translational cup movements; FIG. 7C shows circuitous translational cup movements; FIG. 7D shows rotational (e.g., twisting) cup movements; FIG. 7E shows a particular exemplary sequence of side-to-side translational cup movements; and FIG. 7F shows an exemplary unidirectional translation cup movement;

FIG. 8 is a diagram of images showing gene expression in guinea pig skin after intradermal injections of a plasmid encoding the gene for green fluorescent protein (GFP) and then treated with various techniques and devices each using vacuum treatment;

FIG. 9 is a diagram of images showing gene expression in guinea pig skin after intradermal injections of a plasmid encoding the gene for green fluorescent protein (GFP) and then treated with various kinetic vacuum (KV) treatments (and one static vacuum (SV) treatment) using various vacuum cup designs;

FIGS. 10A and 10B are charts showing binding ELISA immunogenicity data in guinea pigs at two (2) weeks (FIG. 10A) and four (4) weeks (FIG. 10B) after intradermal injections of a plasmid, followed by various treatments, including kinetic vacuum (KV) treatments, static vacuum (SV) treatments, vacuum electroporation (VEP) treatments, and injection-only (INJ) treatments;

FIGS. 10C and 10D contain photographs showing the treatment effects on skin tissue at the treatment sites for the study shown in FIGS. 10A-10B immediately following the treatments (FIG. 10C) and at seven (7) days post-treatment (FIG. 10D).

FIGS. 11A and 11B are charts showing binding ELISA immunogenicity data in guinea pigs at two (2) weeks (FIG. 11A) and four (4) weeks (FIG. 11B) after intradermal injections of a plasmid, followed by various treatments, including kinetic vacuum (KV) treatments, static vacuum (SV) treatments, vacuum electroporation (VEP) treatments, and injection-only (INJ) treatments, as a follow-up to the study shown in FIGS. 10A-10B to confirm the ELISA results thereof;

FIGS. 12A and 12B are charts showing binding ELISA immunogenicity data in guinea pigs at two (2) weeks (FIG. 12A) and four (4) weeks (FIG. 12B) after intradermal injections of a plasmid at different volumes, followed by kinetic vacuum (KV) treatments;

FIG. 13 is a chart showing binding ELISA immunogenicity data in naïve guinea pigs at two (2) weeks after intradermal plasmid injections followed by various treatments, including vacuum electroporation (VEP) treatments, kinetic vacuum (KV) treatments, static vacuum (SV) treatments, and injection only (INJ) treatments;

FIG. 14 is a chart comparing binding ELISA immunogenicity data in guinea pigs at two (2) weeks after intradermal plasmid injections followed by various kinetic vacuum treatments that employed different amounts of cup translations across the injection site, with one such treatment including hyaluronidase;

FIG. 15 is a chart comparing ELISA immunogenicity data in guinea pigs after intradermal plasmid injections followed by various kinetic vacuum (KV) treatments that employed different vacuum pressures;

FIGS. 16A and 16B are charts comparing ELISA immunogenicity data in rabbits at Day 0 (FIG. 16A) and Week 2 (FIG. 16B) after intradermal plasmid injections followed by various treatments, including kinetic vacuum (KV), static vacuum (SV), vacuum electroporation (VEP), needle electroporation (NEP), and injection-only (INJ) treatments;

FIGS. 17A and 17B are charts in a follow-up study to that shown in FIGS. 16A-16B, again comparing ELISA immunogenicity data in rabbits at Day 0 (FIG. 17A) and Week 2 (FIG. 17B) after intradermal plasmid injections followed by various treatments, including kinetic vacuum (KV) treatments;

FIG. 18 is a chart comparing ELISA immunogenicity data in guinea pigs at two (2) weeks after intradermal plasmid injections followed by various vacuum treatments, including kinetic vacuum (KV) treatments and static vacuum (SV) treatments with and without hyaluronidase;

FIG. 19 is a chart comparing ELISA immunogenicity data in guinea pigs at two (2) weeks after treatment for evaluating the impact that the number of kinetic vacuum (KV) cup movements and skin thickness at the treatment site have on immune response;

FIGS. 20A and 20B show gene expression in guinea pig skin after intradermal injections of a plasmid encoding the gene for green fluorescent protein (GFP) according to different injection volumes, both with and without hyaluronidase, followed by kinetic vacuum (KV) or static vacuum (SV) treatments; FIG. 20A is a table of images showing visible GFP expression; FIG. 20B is chart showing quantified values of the measured florescence;

FIG. 21 is a chart comparing ELISA immunogenicity data in guinea pigs at two (2) weeks after treatment for evaluating the impact that high injection volumes with and without hyaluronidase have on immune responses (ELISA expression) produced by kinetic (KV) and static vacuum (SV) treatments;

FIG. 22 is a chart comparing ELISA immunogenicity data in guinea pigs at two (2) weeks after treatment for evaluating the impact that combining kinetic vacuum (KV) treatments with vacuum electroporation (VEP) has on immune responses (ELISA expression);

FIGS. 23A-23D are charts comparing binding ELISA data (FIGS. 23A and 23B), ELISpot data (FIG. 23C), and SARS-COV-2 pseudovirus neutralization data (FIG. 23D) produced by kinetic vacuum (KV) treatments versus mRNA treatments and needle electroporation (NEP) treatments

FIG. 24 is a table of images showing gene expression in guinea pig skin after various treatments involving injection partitioning that employ multi-injection (multi-bleb) sites enhanced by a single kinetic vacuum (KV) treatment.

FIG. 25 is a chart of an operator study showing binding ELISA immunogenicity data in guinea pigs at two (2) weeks after intradermal plasmid injections followed by the same kinetic vacuum (KV) treatments performed by four (4) different individuals (operators);

FIGS. 26A-26C are charts of a nearly one (1) year study comparing binding ELISA data (FIG. 26A) and SARS-COV-2 pseudovirus neutralization data (FIGS. 26B-26B) produced by kinetic vacuum (KV) treatments using DNA-launched nanoparticles (DNLP) versus mRNA treatments;

FIG. 27 is a chart showing comparative spreading effects (measured as a ratio of pre- to post-treatment bleb diameters) for kinetic vacuum (KV) treatments (with and without hyaluronidase added to the injection) and for static vacuum (SV) treatments;

FIG. 28A is a chart showing a vacuum cup device study comparing binding ELISA data in guinea pigs at two (2) weeks receiving the same kinetic vacuum (KV) treatment (e.g., movement pattern) administered by different devices, including a vacuum cup having a centerpost and four (4) off-the-shelf (OTS) vacuum cup devices;

FIG. 28B is a chart comparing binding ELISA data in guinea pigs at two (2) weeks produced by performing the same kinetic vacuum (KV) treatment using different vacuum cup devices having different cup sizes and geometries;

FIG. 29 is a chart comparing binding ELISA data in guinea pigs at two (2) weeks produced by partitioning injection volumes into multiple blebs, followed by performing the same kinetic vacuum (KV) treatments over the partitioned blebs in each group collectively;

FIG. 30 is a chart comparing binding ELISA data in guinea pigs at two (2) weeks produced by injections with reduced DNA plasmid dosages followed by kinetic vacuum (KV) treatments versus needle electroporation (NEP) treatments;

FIG. 31 is a chart comparing binding ELISA data in guinea pigs at two (2) weeks produced by reducing injection volume and DNA dosage across the tested groups, followed by performing the same kinetic vacuum (KV) treatments using the same vacuum cup device for each group;

FIGS. 32A-32E are charts comparing immune responses, particularly ELISA responses (FIGS. 32A-32C) and T-cell responses (FIGS. 32D and 32E), in rabbits following treatments involving kinetic vacuum (KV) versus needle electroporation (NEP) and intramuscular electroporation (IM-EP);

FIG. 33 is a chart showing a kinetic vacuum (KV) movement pattern study, comparing the effect of simplified KV movement patterns and repetitions (cycles) on binding ELISA data in guinea pigs at two (2) weeks;

FIGS. 34A and 34B are charts comparing binding ELISA data (FIG. 34A) and SARS-COV-2 pseudovirus neutralization data (FIG. 34B) produced in pigs by kinetic vacuum (KV) treatments versus needle electroporation (NEP) treatments;

FIGS. 35A and 35B illustrate a study exploring the effect of off-bleb kinetic vacuum (KV) movements on gene expression; FIG. 35A is a plan view showing the kinetic movements of a vacuum cup employed in this study; and FIG. 35B is a chart showing quantified values of visible gene expression in guinea pig skin after intradermal injections of a plasmid encoding the gene for green fluorescent protein (GFP) following similar KV movements progressively spaced further from the injection bleb;

FIGS. 36A-36C are charts comparing binding ELISA data at Week 3 (FIG. 36A) and Week 4 (FIG. 36B) and T-cell responses at Week 4 (FIG. 36V) in mice resulting from uniform injection volumes (and DNA doses) of a plasmid followed by kinetic vacuum (KV) treatments with hyaluronidase versus injection only (INJ), static vacuum (SV), and needle electroporation (NEP) treatments;

FIG. 37 is a chart comparing binding ELISA data at Week 2 in guinea pigs following different dosages of hyaluronidase added to otherwise uniform DNA-dosage injections of a plasmid followed by uniform kinetic vacuum (KV) treatments;

FIG. 38 is a chart showing quantified immune cell migration in guinea pigs following kinetic vacuum (KV) treatment versus needle electroporation (NEP) treatment, particularly showing the number of GFP-positive cells in subject lymph nodes at 3-days post-treatment for KV versus NEP treatment groups;

FIG. 39 is a chart showing binding ELISA data at Week 2 in rabbits following low-DNA-dosage injections of a plasmid followed by kinetic vacuum (KV) treatments, particularly comparing the immunogenic outcomes provided by adding hyaluronidase to the low-dosage injections;

FIG. 40 is a chart showing binding ELISA data at Week 2 in rabbits following medium-DNA-dosage injections of a plasmid followed by kinetic vacuum (KV) treatments, particularly comparing the immunogenic outcomes provided by adding hyaluronidase to the medium-dosage injections;

FIG. 41 is a chart showing quantified immune cell migration in rabbits following kinetic vacuum (KV) treatments, particularly showing the number of GFP-positive cells in subject lymph nodes at Days 1, 2, 3, and 7 post-treatment, thereby indicating the period(s) at which GFP-positive cell migration occurs;

FIG. 42 is a chart showing binding ELISA data at Week 2 in guinea pigs following low-DNA-dosage injections and subsequent kinetic vacuum (KV) treatments, particularly comparing the “tradeoffs” resulting from diluting the low-DNA-dosage injections with various hyaluronidase dosages;

FIG. 43 is a chart comparing the effect on binding ELISA data at Week 2 in guinea pigs produced by adjusting the number of repetitions (cycles) of a kinetic vacuum (KV) movement pattern;

FIG. 44 is a chart comparing the effect on binding ELISA data at Week 2 in guinea pigs provided by combining intradermal (ID) injections of mRNA-1273 (an mRNA vaccine formulated using lipid nanoparticles) at regular and low doses with kinetic vacuum (KV) treatments versus intramuscular (IM) injections of mRNA-1273 at regular and low doses without KV treatments;

FIG. 45 is a chart comparing the effect on binding ELISA data at Week 2 in guinea pigs provided by combining intradermal (ID) injections of mRNA-1273 (an mRNA vaccine formulated using lipid nanoparticles) at regular, medium, and low doses with kinetic vacuum (KV) treatments versus ID injections of mRNA-1273 at regular and low doses without KV treatments; and

FIG. 46 is a chart showing binding ELISA data at Week 2 in guinea pigs following high-DNA-dosage injections and subsequent kinetic vacuum (KV) treatments, particularly comparing the “tradeoffs” resulting from diluting the high-DNA-dosage injections with various hyaluronidase dosages.

### DETAILED DESCRIPTION OF ILLUSTRATIVE EMBODIMENTS

The present disclosure can be understood more readily by reference to the following detailed description taken in connection with the accompanying figures and examples, which form a part of this disclosure. It is to be understood that this disclosure is not limited to the specific devices, methods, applications, conditions, or parameters described and/or shown herein, and that the terminology used herein is for the purpose of describing particular embodiments by way of example only and is not intended to be limiting of the scope of the present disclosure.

Also, as used in the specification including the appended claims, the singular forms “a,” “an,” and “the” include the plural, and reference to a particular numerical value includes at least that particular value, unless the context clearly dictates otherwise.

The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

The terms “approximately”, “about”, and “substantially”, as used herein with respect to dimensions, angles, ratios, and other geometries, takes into account manufacturing tolerances. Further, the terms “approximately”, “about”, and “substantially” can include 10% greater than or less than the stated dimension, ratio, or angle. Further, the terms “approximately”, “about”, and “substantially” can equally apply to the specific value stated.

The term “kinetic vacuum” (KV), as used herein, means a moving field of vacuum pressure, and is caused by moving a vacuum applicator (e.g., a vacuum cup) with respect to a surface of target tissue.

The term “static vacuum” (SV), as used herein, means a field of vacuum pressure that does not move with respect to a surface of target tissue. An example of a static vacuum can include a vacuum cup that applies vacuum pressure to tissue while remaining stationary relative to the tissue.

The term “electroporation” (EP), as used herein, means employing an electrical field within tissue that temporarily and reversibly increases the permeability and/or porosity of the cell membranes of cells in the tissue, thereby allowing an agent to be introduced into the cells.

When used herein as a postposition to a primary reference character, the character “n” (e.g., Pn) indicates that the primary reference character (“P”) can have an open-ended number of counterparts. For example, when referring to various spatial positions P1, P2, P3, etc. shown in the Figures, the combined reference character “Pn” when used below indicates that there can be yet additional positions not shown in the respective Figure(s).

It should be understood that, although terms involving numerical prepositions (e.g., “first,” “second”) can be used herein to describe various features, such features should not be limited by these terms. These terms are instead used to distinguish one feature from another. For example, a first element could be termed a second element in another context, and, similarly, a second element could be termed a first element in another context, without departing from the scope of the embodiments disclosed herein.

The embodiments described herein pertain to systems and devices that perform kinetic vacuum treatment upon tissue, particularly to a targeted layer of tissue, particularly skin tissue, although the treatments herein can be adapted for adipose tissue and/or muscle tissue. These embodiments subject a targeted area of the tissue to a moving field of vacuum pressure (i.e., kinetic vacuum) to impose mechanical stress and strain upon the target tissue (such as by deforming the tissue) in a manner favorable for enhancing agent delivery in vivo, particularly by modes of increasing fluid dispersion (spreading) of the injectate within tissue, increasing cellular uptake of the agent directly into target cells of the tissue (e.g., transfection of nucleic acids), and increasing immune responses to injectates (i.e., injected agents). Accordingly, the embodiments described herein can be said to provide numerous modes for enhancing agent delivery. The agents deliverable by the kinetic vacuum treatments herein include, by way of non-limiting examples, plasmids (e.g., DNA vaccine plasmids), peptides, small molecules, nucleic acids, synthetic DNA-encoded monoclonal antibodies (DMAbs), synthetic DNA-encoded proteins, cancer antigens, viral antigens associated with chronic infection, bacterial or other microorganism antigens or proteins, and combinations thereof.

In the embodiments herein, an open end of a vacuum device, such as a vacuum cup, is placed in contact with an outer surface of the tissue (e.g., the “skin”) overlying a tissue volume, vacuum pressure is applied to an interior of the cup, thereby drawing a portion of the tissue within the target area into the vacuum cup and momentarily holding the tissue portion within the cup. While the vacuum field is applied to the tissue, the cup is moved relative to the tissue surface, which imparts mechanical stresses and strains (i.e., deformations) to at least some of the tissue that is momentarily and/or reciprocally drawn into the vacuum cup.

The inventors have observed that such kinetic vacuum treatments generate a predictable, substantially uniform zone of cellular uptake (e.g., transfection zone) within the target tissue area. The kinetic vacuum treatments provided by the embodiments described below have also been observed to provide favorable redistribution of fluid within the target tissue area, including favorable in vivo dispersion of an injectate within the tissue volume, and also favorable in vivo ingress and egress of fluid into and out of the target zone. For example, the kinetic vacuum treatments herein have been observed to enhance the dispersion of the injectate throughout the tissue to enlarge the transfection zone and also draw more in vivo fluids into the target tissue area, increasing the amount of cells that are exposed to transfected cells.

The inventors have observed that the kinetic vacuum treatments described throughout this disclosure have resulted in subjects' increased responses to injectates. Compared to a “static vacuum” procedure, wherein the vacuum pressure is applied and then removed without translating or rotating or otherwise moving the vacuum applicator, kinetic vacuum procedures generate stronger and broader transfection of nucleic acids, broader dispersion of injectate throughout the target tissue, and stronger resulting immune responses when the injectate is a vaccine, particularly a nucleic acid vaccine.

The inventors have also observed, surprisingly and unexpectedly, that the kinetic vacuum treatments described herein, without use of electroporation, can cause cellular uptake and immune responses that are comparable with, and can even surpass, those resulting from treatments involving electroporation. While wishing not to be bound by any particular theory, the inventors believe that the vacuum pressure gradient fluctuations and movements imparted by the kinetic use of the vacuum cup (i.e., moving the vacuum pressure field) imparts mechanical stresses and strains on the cell membranes within the tissue volume that increases cell membrane permeability and thus the observed cellular uptake within the tissue volume. The inventors further believe that the aforementioned fluid redistribution and mechanical stresses likely interact with one another to create a favorable environment within the tissue volume for cellular uptake (e.g., transfection) of external agents directly into the cells. The use of kinetic vacuum also appears to uniquely benefit from the addition of a “spreading agent” (for example, hyaluronidase) that permits injectate to flow more freely throughout the tissue, which the inventors believe is due to the aforementioned fluid redistribution capabilities of kinetic vacuum. For example, the inventors have observed that adding hyaluronidase to an injectate dramatically enhances the effect kinetic vacuum has on increasing injectate dispersion throughout skin tissue.

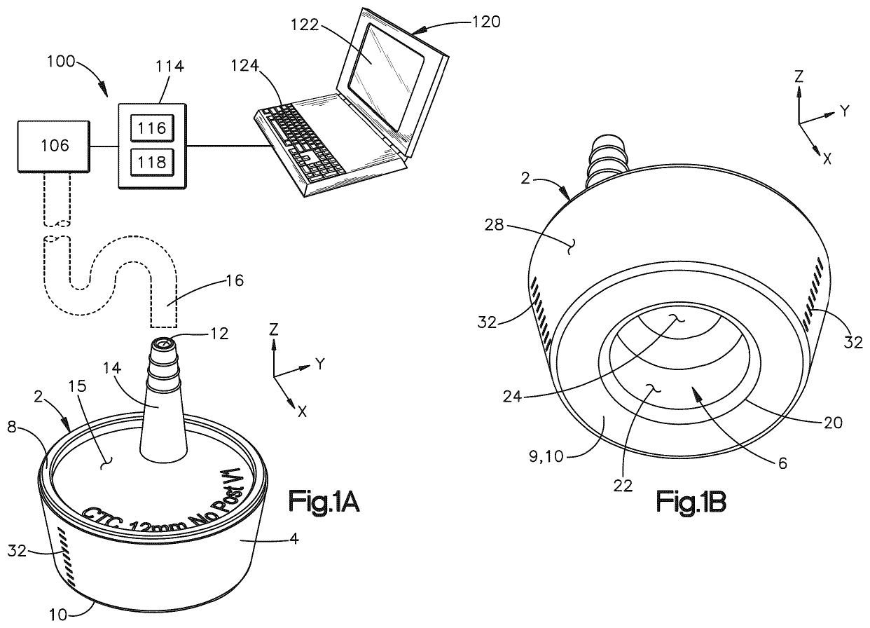

Referring to FIGS. 1A-1B, a vacuum treatment system 100 for treating a patient according to the present disclosure includes a vacuum applicator 2, which includes a housing body 4 that defines an internal vacuum chamber 6. The vacuum applicator 2 can also be referred to as a “vacuum cup” or simply a “cup.” The housing body 4 can be referred to as a “cup housing”. The cup housing 4 extends from a proximal end 8 to a distal end 9 along a transverse direction Z. The distal end 9 is spaced from the proximal end 8 in a distal direction D along the transverse direction Z, while the proximal end 8 is spaced from the distal end 9 in a proximal direction P that also extends along the transverse direction Z and is opposite the distal direction D. It should be appreciated that the proximal and distal directions P, D are mono-directional components of the transverse direction Z, which is bi-directional. The cup housing 4 also defines a central axis Z1 oriented along the transverse direction Z. The central axis Z1 can also be characterized as a central axis of the vacuum chamber 6 and/or a central axis of the vacuum cup 2.

The vacuum cup 2 is configured so that a user (such as a physician) can place a distal end surface 10 of the vacuum cup 2 at the distal end 9 thereof onto an outer surface of tissue (e.g., skin) targeted for treatment, such as a portion of the skin surface overlying a liquid injection (that was previously injected into the tissue). At such vacuum cup 2 position on the skin surface, the user can apply vacuum pressure to the vacuum chamber 6 to draw, pull, or otherwise induct the tissue (e.g., skin tissue) into the vacuum chamber 6. With tissue drawn into the vacuum chamber 6, the user can move the vacuum cup 2 along the tissue surface, thereby manipulating the tissue along the cup's path of travel, which manipulation is referred to herein as “kinetic vacuum treatment.” The target tissue manipulable according to the kinetic vacuum treatments described below include the dermal layers (epidermis and dermis) and can include additional layers, such as subcutaneous fat (i.e., the adipose layer), as described in more detail below.

The cup housing 4 can include a proximal surface 15 at or adjacent the proximal end 8. In the illustrated embodiment, the cup housing 4 includes a peripheral, annular lip 17 that extends proximally from the proximal surface 15 to the proximal end 8. In other embodiments, the cup housing 4 need not include the peripheral, annular lip 17, such that the proximal surface 15 also defines the proximal end 8 of the cup housing 4. Additionally, the proximal surface 15 can be substantially planar, as shown, although other surface geometries are within the scope of the present disclosure.

The vacuum cup 2 includes one or more couplings, such as ports, for connection to one or more external components. For example, the vacuum cup 2 has a port 12 for providing fluid communication between the vacuum chamber 6 and a vacuum source 106, such as a vacuum pump. The port 12 can be defined along a port coupling 14, such as a stem 14, for connection to tubing 16 that provides fluid communication between the vacuum chamber 6 and the vacuum source 106. The stem 14 can extend proximally from the proximal surface 15 of the housing body 4. The vacuum source 106 can be in electrical communication with a control unit 114 (also referred to herein as a “controller”), which can include a processor 116 configured to control operation of the vacuum treatment system 100, including operation of the vacuum source 106. The processor 116 can be in electronic communication with computer memory 118, and can be configured to execute software and/or firmware including one or more algorithms for controlling operation of the system 100. The processor 116 can also be in electrical communication with a user interface 120, which can include a display 122 for presenting information relating to operation of the system 100 and a keypad 124 allowing an operator, such as user, to input information, such as commands, relating to operation of the system 100. It should be appreciated that the display 122 can be a touchscreen display allowing the operator to input information directly at the display 122. It should also be appreciated that the interface 120 can be computer interface, such as a table-top computer or laptop computer, or a hand-held electronic device, such as a smart-phone or the like.

Referring now to FIGS. 1B-1C, the distal end 9 of the vacuum cup 2 defines at least one opening 20 leading into the vacuum chamber 6. The opening 20 can be circular as shown, although other opening shapes are within the scope of the present disclosure. The distal end 9 of the vacuum cup 2 (and thus also the opening 20) can be defined by the housing 4. In the illustrated embodiment, the distal end 9 of the vacuum cup 2 also defines the distal end of the vacuum chamber 6. Within the vacuum chamber 6, the housing 4 defines an interior surface 22 that extends from the distal end surface 10 of the housing 4 to a proximal end surface 24 within the chamber 6. The proximal end surface 24 can define a proximal end 26 of the chamber 6. In the illustrated embodiment, the chamber 6 is defined by the interior surface 22 and the proximal end surface 24. Additionally, the vacuum cup 2 of the present embodiment can be referred to as a “dome vacuum cup” 2 because of the generally dome-like geometry of the vacuum chamber 6. The chamber 6 has a chamber diameter D1 measured between opposing portions of the interior surface 22 along a radial direction R that is perpendicular to and intersects the central axis Z1. The chamber 6 also defines a chamber depth L1 measured from the distal end 9 to the proximal end 26 of the chamber 6 along the transverse direction Z. The chamber diameter D1 can be in a range from about 1.0 mm to about 50.0 mm, more particularly in a range of about 3.0 mm to about 20.0 mm, and more particularly in a range from about 5.0 mm to about 15.0 mm. The chamber depth L1 can be in a range from about 1.0 mm to about 50.0 mm, more particularly in a range of about 3 mm to about 20 mm, and more particularly in a range from about 5 mm to about 17 mm.

The housing 4 also defines an exterior surface 28 that is spaced from the interior surface 22 along the radial direction R. The housing 4 also defines a wall 30 that extends from the interior surface 22 to the exterior surface 28 of the housing 4 along the radial direction R. In the illustrated embodiment, the housing 4 has a substantially circular geometry in a reference plane that extends along first and second directions X, Y that are perpendicular to each other and also perpendicular to the transverse direction Z. Accordingly, the interior and exterior surfaces 22, 28 and the wall 30 can revolve uniformly about the central axis Z1 in circumferential fashion, as shown. It should be appreciated, however, that other housing geometries are within the scope of the present disclosure, including the housing (and chamber) geometries described in U.S. Patent Publication No. 2021/0290941 A1, published Sep. 23, 2021, entitled “VACUUM-ASSISTED ELECTROPORATION DEVICES, AND RELATED SYSTEMS AND METHODS” (hereinafter, “the '941 Reference”), the entire disclosure of which is hereby incorporated by reference as if set forth in its entirety herein.

The housing 4 can be formed of a material that is preferably transparent or semi-transparent, thereby allowing visualization of tissue drawn into the vacuum chamber 6 during use. As shown, the housing 4 can include graduations 32 along the exterior surface 28 and configured for providing a visual indication of the depth at which tissue is drawn into the chamber 6. The housing 4 material also can have a measure of flexibility, particularly via elastic deformation, which can reduce patient discomfort during use. The housing 4 material can be a polymeric material, including polyetheretherketones (PEEK), polyphthalamides (PPA), polyethylenes, polycarbonates, polytherimides (PEI), polyvinyl chlorides (PVC), polytetra-fluoroethylenes (PTFE), polyamides, polyimides, polysiloxanes (silicone), polyethylene terephthalates, polyurethanes, crosslinked or non-crosslinked rubbers (elastomers), polyesters, by way of non-limiting examples. It should be appreciated that other bio-compatible and/or medical-grade materials can be employed for the housing 4. The housing 4 can be a monolithic structure that defines the vacuum cup 2, as in the embodiments illustrated herein. In other embodiments, however, the housing 4 need not be a monolithic structure and can instead include two or more body components coupled together to define the housing 4. Additionally, the distal end surface 10 of the vacuum cup 2 can be subjected to one or more finishing processes for reducing the surface finish roughness or otherwise smoothening and/or polishing the distal end surface 10, thereby reducing friction with the skin surface during kinetic vacuum treatments, and thereby improving patient comfort during and after treatment.

Referring now to FIG. 1C, the interior surface 22 preferably has a generally bell-shaped geometry. The interior surface 22 has a primary surface portion 22a that extends intermediate the distal and proximal ends 10, 26 of the chamber 6. The primary surface portion 22a can have a linear profile in an axial reference plane, although other profile geometries are within the scope of the present disclosure. As shown in FIG. 1C, the primary surface portion 22a can have a linear profile that is oriented at an acute taper angle A0 relative to a linear axis oriented along the transverse direction Z in the axial reference plane. In this manner, the inner diameter of the chamber 6 diminishes toward the proximal end 26. The taper angle A0 can be in a range of about 0 degrees to about 60 degrees, and more particularly in a range of about 0.5 degrees to about 15 degrees, and more particularly in a range of about 5 degrees to about 10 degrees.

The interior surface 22 also preferably includes a distal lead-in portion 22b that extends from the distal end surface 10 to the primary surface portion 22a. The distal lead-in portion 22b preferably has a tapered, radiused contour for reducing or otherwise mitigating tissue damage, such as bruising, at the periphery of tissue draw into the chamber 6 during use of the vacuum cup 2. In the embodiments described herein, the chamber diameter D1 is measured at the interface between the distal lead-in portion 22b and the primary surface portion 22a. The chamber diameter D1 can optionally be measured at other locations along the interior surface 22. The interior surface 22 can include a proximal relief portion 22c that extends from the primary surface portion 22a to the proximal end surface 24. The proximal relief surface 22c is preferably radiused or otherwise shaped to reduce stress concentrations within the housing 4 during use. Interfaces between the primary surface portion 22a and the distal lead-in portion 22b and the proximal relief surface 22c are indicated in FIG. 1C by reference marks 23.

The distal end 9 of the vacuum cup 2 also preferably has a rounded or otherwise chamfered exterior relief surface 29 that extends between the distal end surface 10 and the exterior surface 28. In this manner, the exterior relief surface 29 is configured to reduce friction and/or abrasion (e.g., scraping) with the skin at the outer edge of the cup 2 during kinetic vacuum cup movements, thereby enhancing patient comfort. Interfaces between the exterior relief surface 29 and the distal end surface 10 and exterior surface 28 of the cup 2 are indicated in FIG. 1C by reference marks 25. The cup wall 30 defines a wall thickness W1 between the interior and exterior surfaces 22, 28. In the embodiments described herein, the wall thickness W1 is measured along the radial direction R between: (1) the interface 23 between the distal lead-in portion 22b and the primary surface portion 22a; and (2) the interface 25 between the exterior relief surface 29 and the exterior surface 28. The wall thickness W1 can optionally be measured at other locations along the wall 30. It should be appreciated that the exterior surface 28 of the vacuum cup 2, particularly at the interface 25 with the exterior relief surface 29, can be employed as a visual reference when performing kinetic vacuum cup movements. For example, this interface 25 can be used to visually determine when the vacuum cup 2 has translated across and/or off the injection site, as described in more detail below.

Referring now to FIG. 1D, the vacuum cup 2 is configured for placement atop a tissue surface, such as a surface 3 of skin tissue 1, and overlying a fluid injectate 7 in the tissue, with the distal end surface 10 of the cup 2 in contact with the tissue surface 3 or with an intermediary substance, such as a gel, oil, lotion, or lubricant for facilitating smooth cup movement along the tissue surface 3. With the vacuum cup 2 positioned in this manner, vacuum pressure can be supplied to the chamber 6 via the port 12 at sufficient magnitude to draw the tissue 1 into the chamber 6. As shown, the tissue 1 can assume a mound-like or domed shape within the chamber 6 responsive to the vacuum pressure. The tissue can be drawn into the chamber 6 to various depths L2, as measured from an apex of the tissue to the distal end 9 of the chamber 6, responsive to the magnitude of vacuum pressure communicated to the chamber 6. The vacuum pressure communicated to the chamber 6 can be in a range of about −0.1 psi to about −14.7 psi (about −0.70 kPA to about −100 kPA) (about −5 mmHg to about −760 mmHg), and more particularly in a range of about −3 psi to about −14.7 psi (about −20 kPA to about −100 kPA) (about −155 mmHg to about −760 mmHg), and more particularly in a range of −7.7 psi to about −14.7 psi (about −53 kPa to about −100 kPa) (about −400 mmHg to about −760 mmHg), and yet more particularly in a range of about −7.7 psi to about −11.6 psi (about −53 kPa to about −80 kPA) (about −400 mmHg to about −600 mmHg).

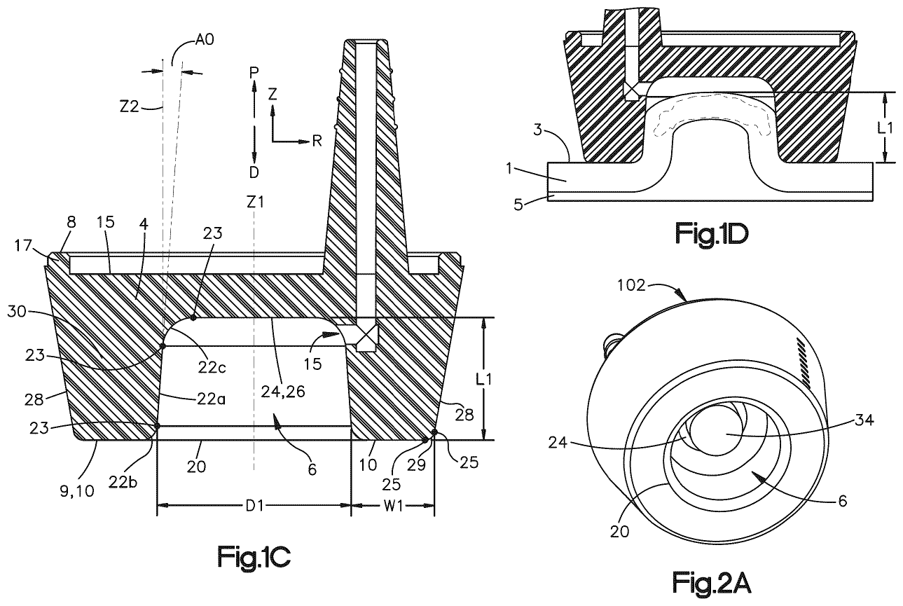

Referring now to FIGS. 2A-2C, another embodiment of a vacuum cup 102 is shown having a different interior vacuum chamber geometry than that of the vacuum cup 2 described above. In particular, the vacuum cup 102 of the present embodiment includes a protrusion 34 that extends within the chamber 6 from the proximal end surface 24 toward the opening 20. The vacuum cup 102 of the present embodiment can be otherwise similar to the vacuum cup 2 described above. Accordingly, features of the vacuum cup 102 that are similar to those of the vacuum cup 2 described above can use the same reference characters as above. The protrusion 34 preferably extends centrally along the central axis Z1. Thus, the protrusion 34 can be referred to herein as a “centerpost” 34. Additionally, the vacuum cup 102 of the present embodiment can be referred to as a “centerpost vacuum cup” 102.

As shown in FIG. 2B, the centerpost 34 has a length L3 measured from the proximal end surface 24 to a distal end 36 of the post 34. The vacuum cup 102 also defines a post depth L4 measured from the distal end 9 of the chamber 6 to the distal end 36 of the post 34. The centerpost 34 of the illustrated embodiment has a rounded distal end geometry. For example, as shown, the centerpost 34 has a rounded distal end surface having a radius R1. As shown in FIG. 2C, the centerpost 34 can be configured to contact the surface 3 of the tissue drawn into the chamber 6 and impart additional mechanical stress and strain to the injected tissue, such as by deforming the tissue that contacts the centerpost 34, which can lead to better treatment results, as described in more detail below. It should be appreciated that various other geometrical formations can be employed within the chamber 6 for increasing mechanical stress and strain on the tissue draw in to the chamber 6. For example, the centerpost 34 can have a textured outer surface (e.g., bumps and/or dimples). Additionally or alternatively, a vacuum cup can have a plurality of posts that extend distally within the chamber 6, which posts can have various sizes and geometries and can be arrayed in various patterns. Additionally or alternatively, a vacuum cup can have one or more posts that can move with respect to the cup housing 4, such as by being compressible or extensible, such as along the transverse direction Z.

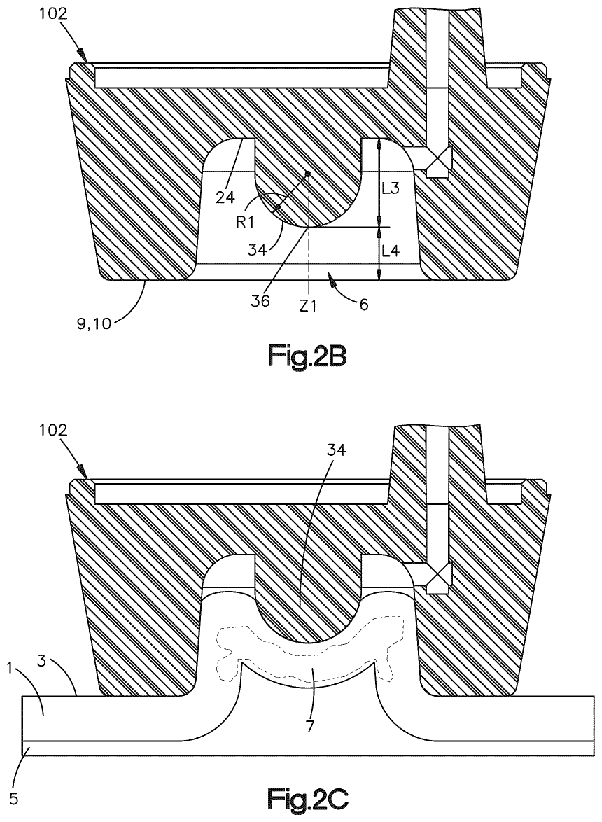

Referring now to FIG. 3A, an additional embodiment of a vacuum cup 202 has an injection channel 38 extending through the cup housing 4 and into the vacuum chamber 6. Such vacuum cups 202 can be referred to as “injection channel vacuum cups” 202. The injection channel 38 is configured for passage of an injectate from a proximal side of the vacuum cup 202, through the cup housing, and into the vacuum chamber 6. The injection channel 38 can be centrally located along the central axis Z1 of the respective vacuum cup 202, as shown, or can be offset from the central axis.

The vacuum cup 202 can be similar to the vacuum cup 2 described above with reference to FIGS. 1A-1D, with the injection channel 38 configured for passage of an injection needle 40 into the vacuum chamber 6. In this embodiment, the vacuum cup 202 preferably includes a septum 42 or other device to form a seal with the injection needle 40 while the needle 40 extends into the vacuum chamber 6. In this manner, the septum 42 or other device can maintain the desired vacuum pressure within the chamber 6 while the injection needle 40 extends through the injection channel 38.

Referring now to FIG. 3B, in further embodiments, a vacuum cup 302 can be configured for use with a jet injector 44, and can otherwise be similar to the vacuum cups 2, 102 described above. In the present embodiment, the injection channel 38 is wide enough to receive a nozzle 48 of the jet injector 44. As shown, the nozzle 48 can extend into the vacuum chamber 6 and can optionally be positioned to contact tissue drawn into the vacuum chamber 6. In this manner, the nozzle 48 can deform the tissue drawn into the chamber 6 in similar fashion to the centerposts 34 described above. In the present embodiment, the vacuum cup 302 can include a mount or other feature for guided coupling with the jet injector 44.

It should be appreciated that the vacuum cups 202, 302 shown in FIGS. 3A-3B can be adapted such that their injection channels 38 are configured for use with other types of injection devices. For example, the vacuum cup 202 shown in FIG. 3A can be adapted such that the injection channel 38 is configured for use with a jet injector or other type of injection device. Similarly, the vacuum cup 302 shown in FIG. 3B can be adapted such that the injection channel 38 is configured for use with an injection needle or other type of injection device.

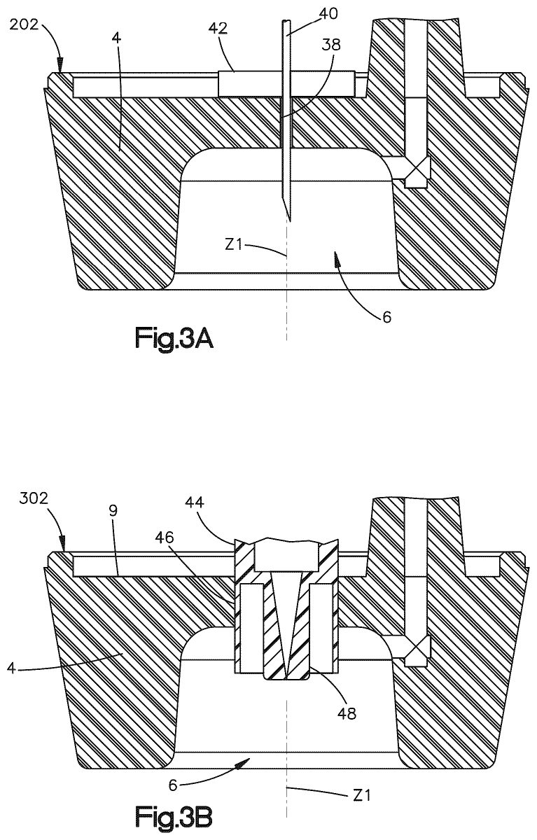

Referring now to FIGS. 4A-4B, another embodiment of a vacuum cup 402 is shown having one or more electrodes for electroporating tissue during a vacuum treatment, which treatment can be referred to herein as “vacuum electroporation” (VEP) treatment. Thus, the vacuum cup 402 of the present embodiment can be referred to herein as a VEP cup 402. The VEP cup 402 of the illustrated embodiment was used primarily for testing to compare treatment outcomes resulting from KV and SV treatments with VEP treatments. It should be appreciated, however, that the VEP cup 402 can be employed to provide kinetic and/or static VEP treatments within the scope of the present disclosure.

The VEP cup 402 of the present embodiment is similar to the vacuum cup 102 disclosed above with reference to FIGS. 2A-2C, with one difference being that the VEP cup 402 has a center electrode 50 at the centerpost 34 and a concentric ring electrode 52 at the distal end 9 of the cup 402. Accordingly, the VEP cup 402 of the present embodiment can also be referred to as a “centerpost-electrode VEP cup” 402. The center and ring electrodes 50, 52 are configured to deliver one or more electroporative pulses to tissue drawn into the vacuum chamber 6 during a vacuum treatment. The electrical parameters of the one or more electroporative pulses (e.g., electrical potential (voltage), electrical current magnitude (amperage), pulse duration, inter-pulse delay, and quantity of pulses) can include those more fully described in the '941 Reference.

The ring electrode 52 of the illustrated example extends inwardly from a first, outer electrode end 54, located near an interface with the exterior surface 28 of the cup housing 4, to a second, inner electrode end 56 located on the primary surface portion 22a of the interior surface 22. Thus, the ring electrode 52 can extend along the distal end 9 and the interior surface 22 of the VEP cup 402 and can also define the distal end surface 10 of the vacuum cup 402. A transmission member, such as a wire or conductive post 55, extends from the ring electrode 52 to an exterior of the cup housing 4 and is configured to transmit the one or more electroporative pulses to the ring electrode 52. As shown, the conductive post 55 can extend proximally through the cup housing 4 to a contact 57 at the proximal surface 15. The center electrode 38 of the illustrated embodiment defines the centerpost 34, although in other embodiments the center electrode 38 can extend along an exterior surface of the centerpost 34. A proximal end of the center electrode 38 defines a contact 59, which can be located at the proximal surface 15 of the cup housing 4. It should be appreciated that, in other embodiments, a VEP cup can employ various other electrode configurations, including any of those described in the '941 Reference. It should also be appreciated that the VEP cup 402 can be configured with an injection channel 38 for an injection needle 40 and/or a jet-injector 44, similar to the vacuum cups 202, 302 described above with reference to FIGS. 3A-3B.

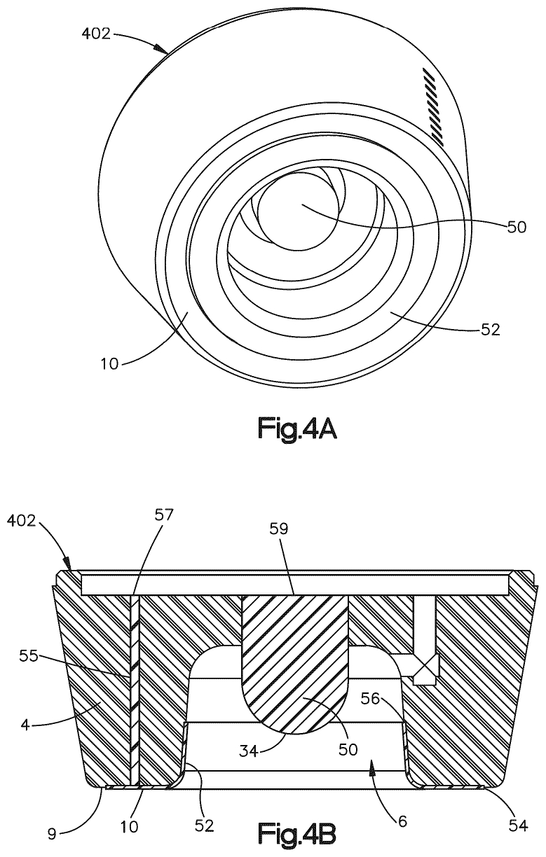

Referring now to FIGS. 5A-5B, any of the vacuum cups 2, 102, 202, 302, 402 described above can be adapted for attachment to a handle assembly 58 of the vacuum treatment system 100. For illustrative purposes, the handle assembly 58 is shown coupled to the vacuum cup 402 disclosed above with reference to FIGS. 4A-4B. The handle assembly 58 includes a handle member 60 having a first, distal end 62 and a second, proximal end 64 opposite the distal end 62. At the distal end 62, the handle member 60 has a mounting formation 66 that is configured to releasably couple with the proximal end 8 of a vacuum cup. For example, one or more an up to each of the vacuum cups 2, 102, 202, 302, 402 described herein can be configured to have a complimentary mounting structure 68 that is configured to releasably couple with (i.e., repeatedly and non-destructively couple with and decouple from) the mounting formation 66 of the handle assembly 58. In this manner, the handle assembly 58 can be configured to interchangeably couple with one or more an up to all of the vacuum cups 2, 102, 202, 302, 402 described herein. The handle assembly 58 can also include at least one button or trigger 67 that can be configured to control operation of the vacuum cup 402, such as for controlling commencement and termination of the application of vacuum pressure within the vacuum chamber 6, by way of a non-limiting example.

Referring now to FIG. 5B, an exemplary handle mounting formation 66 and complimentary cup mounting structure 68 will now be described. For illustrative purposes, the vacuum cup 402 is rotated into a bottom view, while the depicted portion of the handle member 60 remains in side view. The handle mounting formation 66 can include a base surface 70 and a peripheral landing surface 72 recessed proximally from the base surface 70. The peripheral landing surface 72 extends annularly along a periphery of the handle mounting formation 66. The base surface 70 of the handle mounting formation 66 is configured to interface with the proximal surface 15 of the vacuum cup 402 when the cup 402 is attached to the handle mounting formation 66. Additionally, when the cup 402 is attached to the handle mounting formation 66, the peripheral landing surface 72 of the handle mounting formation 66 is configured to interface with the proximal end 8 of the cup 402, with the cup peripheral, annular lip 17 extending proximally from the base surface 70 to the peripheral landing surface 72. In this manner, mating engagement between the cup peripheral, annular lip 17 and the peripheral landing surface 72 can provide a centering mechanism for the vacuum cup 402 relative to the handle mounting formation 66.

The handle mounting formation 66 includes one or more attachment members 74, such as attachment prongs 74, that extend from the base surface 70 and are configured to attach with one or more complimentary attachment formations 76, such as keyed slots 76, of the cup mounting structure 68. The keyed slots 76 are defined in the cup housing 4 along the proximal surface 15 and each have a first, wide slot portion 78 and a second, narrow slot portion 80 extending from the respective wide slot portion 78 in circumferential fashion. The attachment prongs 74 have an extension portion 82 and a latch portion 84 that protrudes laterally from the extension portion 82. During cup 402 coupling with the handle assembly 58, the latch portions 84 of the attachment prongs 74 are configured to advance distally through the wide slot portions 78 of the keyed slots 76 (while the cup peripheral, annular lip 17 is advanced to the peripheral landing surface 72 of the handle mounting formation 66). Subsequently, the vacuum cup 402 is rotated about the central axis Z1 such that the latch portions 84 overhang the housing body 4 alongside the narrow slot portions 80, thereby providing mechanical interference between the latch portions 84 and the housing body 4 in the proximal direction P in a manner maintaining attachment of the cup 402 to the handle mounting structure 66. To decouple the vacuum cup 402 from the handle mounting structure 66, the vacuum cup 402 is rotated in the opposite direction about the central axis Z1 until the latch portions 84 are aligned with the wide slot portions 78, and the cup 402 can then be moved in the distal direction D away from the handle mounting structure 66.

With continued reference to FIG. 5B, the handle member 58 includes a vacuum channel 86 configured to couple with, and provide fluid communication with, the port 12 of the vacuum cup 402 for communicating vacuum pressure to the vacuum chamber 6 when the vacuum cup 402 is coupled with the handle assembly 58. A distal end of the vacuum channel 86 and a proximal end of the port 12 can have one or more complimentary port coupling members for providing a sealed port connection therebetween when the vacuum cup 402 is coupled with the handle assembly 58.

Additionally, as shown, the handle assembly 58 can be adapted for use with VEP cups 402. Accordingly, the handle mounting structure 66 can include one or more electrical contacts 88, 90 configured for providing electrical communication with the contacts 57, 59 of the vacuum cup 402 when the vacuum cup 402 is coupled with the handle assembly 58. As shown, the handle mounting structure 66 can include a first contact 88 for contacting the contact 59 of the center electrode 50 and a second contact 90 for contacting the contact 57 of the ring electrode 52. The contacts 88, 90 of the handle assembly 58 can be in electrical communication with electronic circuitry, which can include a printed circuit board (PCB) 92, which can be in electronic communication with an electronic control device for controlling the one or more electroporative pulses, such as the controller 114, which can be located on-board or off-board the handle assembly 58.

Referring again to FIG. 5A, the handle assembly 58 can include one or more conduits 94 extending away from the handle member 60. The one or more conduits 94 can include tubing 16 that provides fluid communication between the vacuum channel 86 (and thus also the vacuum chamber 6) and the vacuum source 106. The one or more conduits 94 can also include electrical cable(s) for providing electrical communication between the handle assembly 58 and an off-board electrical device.

It should be appreciated that the handle assembly 58 and its handle mounting formation 66 and the complimentary mounting structure 68 of the vacuum cup described above are offered as non-limiting examples of such components and features, and that various other such designs and configurations are within the scope of the present disclosure.

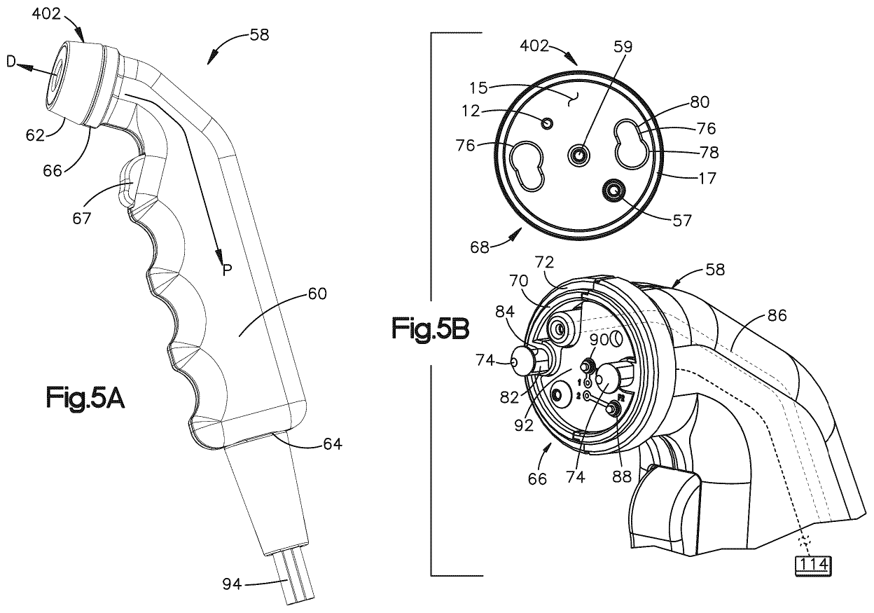

Referring now to FIGS. 6A-6G, a non-limiting example of a kinetic vacuum treatment will now be described. In this example, the kinetic vacuum treatment is shown performed by the vacuum cup 2 described above with reference to FIGS. 1A-1D, although it should be appreciated that similar vacuum treatments can employ any of the other vacuum cups 102, 202, 302, 402 described above. For illustrative purposes, the tissue in FIGS. 6A-6G has graduated markings (−4 to +4) superimposed thereon to provide a visual reference for the cup movements and distances shown in this non-limiting example of kinetic vacuum treatment. Each interval of these graduated markings is equivalent to the radius R2 of the vacuum chamber 6 (R2 =½ D1).

As shown in FIG. 6A, a fluid injectate containing a drug is delivered to intradermal tissue 1 (i.e., skin tissue) by an injection needle 40, such as via Mantoux injection, which creates a bolus of the injected fluid 7 in the intradermal tissue below the skin surface 3. The bolus of injection fluid 7 can cause a lump or “bleb” 11 to form on the skin surface 3. As shown in FIG. 6B, after the injection, the needle 40 is removed and, optionally, a substance 13 can be applied to the skin surface 3 along the target treatment site around the injected fluid 7 to facilitate smooth cup movement along the skin surface 3 during the kinetic vacuum treatment. The substance 13 can be a gel (e.g., ultrasound gel), oil, lotion, or other lubricant.

As shown in FIG. 6C, the vacuum cup 2 can be placed atop the substance 13 on the skin surface 3 at a first position P1 adjacent the injected fluid 7. In this example, the first position P1 is centered over the injected fluid 7 (i.e., the bleb 11). Alternatively, in the first position P1, the cup 2 can be placed at least partially over the bleb 11 but not centered thereupon, or can be placed entirely offset from the bleb 11.

As shown in FIG. 6D, vacuum pressure can be supplied to the vacuum chamber 6, thereby applying a vacuum field to the tissue (particularly the skin surface 3) beneath the vacuum chamber 6 sufficient to draw the tissue into the chamber 6. In this non-limiting example, the vacuum pressure can be supplied at about −500 mmHG (about −66.7 kPa). In the illustrated example, the tissue drawn into the chamber 6 includes intradermal tissue 1 (e.g., epidermis and dermis) and some subcutaneous tissue 5. It should be appreciated that the amount and type of tissue drawn into the chamber 6 can be determined by various factors, including the supplied vacuum pressure.

As shown in FIG. 6E, the vacuum cup 2 is translated at a first distance in a first direction along the skin surface 3 from the first position P1 to a second position P1, thereby also effectively pulling underlying tissue into and out of the chamber 6 as the chamber 6 passes overhead. As shown in FIG. 6F, the vacuum cup 2 is translated at a second distance in a second direction opposite the first direction along the skin surface 3 from the second position P2 to a third position P3, again effectively pulling underlying tissue into and out of the chamber 6 as the chamber 6 passes overhead. It should be appreciated that the vacuum cup 2 can be translated back-and-forth across the skin surface 3 between the positions P1-P3 shown in FIGS. 6E and 6F numerous times, including three (3) or more times, by way of a non-limiting example. As shown in FIG. 6G, the vacuum cup 2 can be removed from the skin surface 3 and the treatment can be completed.

As shown in the foregoing example, the translation distance from the first position P1 to the second position P2 is substantially equivalent to the chamber diameter D1 of the cup 2, thereby causing the trailing side of the interior surface 18 at P2 to assume the position that the leading side of the interior surface 18 occupied at P1. Thus, if the bleb 11 width is substantially equal to the chamber diameter D1 and the first position P1 is centered over the bleb 11, such movement from P1 to P2 causes the trailing side of the interior surface 18 to move from one lateral edge of the bleb 11 to the opposite lateral edge of the bleb 11. In these conditions, such movement also causes the chamber 6 to transition from substantially entirely encompassing the bleb 11 at P1 to being substantially entirely offset from the bleb 11 at P2 and P3. Thus, such movements at these distances can effectively cause the edges of the chamber 6 to transition between opposite edges of the bleb 11. In this manner, the user can reference or “index” the kinetic vacuum cup movements by the spatial relationship between the interior surface 18 and the edges of the bleb 11. Accordingly, during each translation in this example, the user can employ the trailing edge of the interior surface 18 as a reference for when to stop the translation. In embodiments where the housing body 4 is transparent or semi-transparent, the interior surface 18 can be used as a visual reference governing the translation.

Referring now to FIG. 6H, in another example of a kinetic vacuum treatment, the exterior surface 28 of the vacuum cup 2 can be employed as a visual reference governing the cup translation. For illustrative purposes, the tissue in FIG. 6H has graduated markings (−6 to +6) superimposed thereon to provide a visual reference for the exemplary cup movements and distances. In this example, the cup 2 is translated from a first position P1 centered in the bleb 11 to a second position P2 that is offset from the bleb 11, then to a third position P3 offset from the bleb 11 on the opposite side thereof, then back to a fourth position P4 that is coincident with the first position P1. At the offset positions P2, P3 of this example, the exterior surface 28 of the vacuum cup 28 spaced from the near-edge of the bleb 11 by a distance substantially equivalent to one of the graduated intervals (i.e., ½ D1). In other examples, the offset positions P2, P3 can be such that, at these positions, the near side of the exterior surface 28 of the cup 2 is substantially aligned with the proximate edge of the bleb 11. In such other examples, the user can translate the cup 2 from P1 to P2 and from P2 to P3 until the trailing side of the exterior surface 28 reaches the opposite edge of the bleb 11, as guided by visual observation. In these examples discussed with reference to FIG. 6H, the translation distances between the positions P1-P4 is determined by the width of the bleb 11 and the wall thickness W1 of the vacuum cup 2.

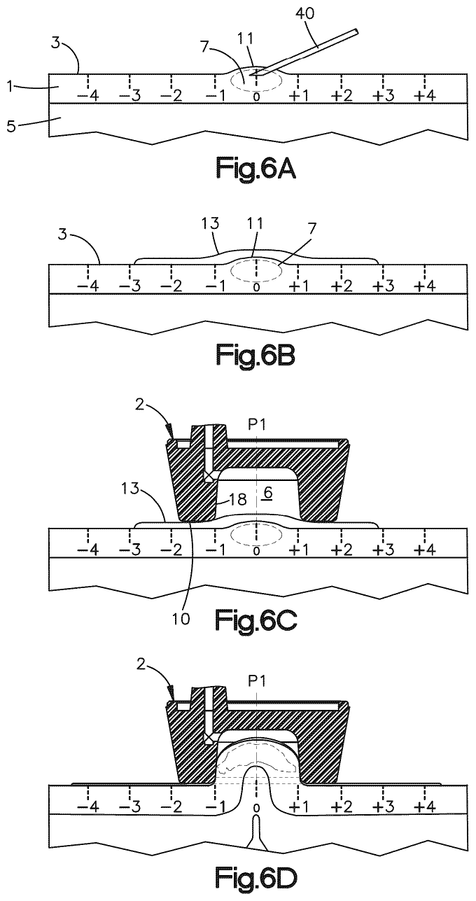

With reference to FIGS. 7A-7D, example cup movements will be described according to various techniques for providing kinetic vacuum treatments. It should be appreciated that the illustrated examples shown in these Figures are provided for illustrative purposes and for discussion of exemplary types of kinetic vacuum cup movements within the scope of the present disclosure. It should also be appreciated that these kinetic vacuum treatments are indicated in FIGS. 7A-7D by circles labeled with reference numeral 2z, which generally represent a vacuum cup 2z at the various positions described. In these illustrated examples, the references circles 2z more precisely represent the position of the vacuum chamber 6, thus these exemplary illustrated movements are indexed to the cup interior surface 18 that defines the lateral bounds of the chamber 6. However, as mentioned above, the cup movements can alternatively be indexed to the exterior surface 28 of the cup 2, in which case the reference circles 2z shown in FIGS. 7A-7D can analogously represent the exterior surface 28 of the cup 2z, depending upon how the user decides to index the cup positions. It should also be appreciated that the vacuum cup 2z shown in these examples represents any of the vacuum cups 2, 102, 202, 302, 402 described herein.

Referring now to FIGS. 7A-7B, example kinetic vacuum cup movements includes translating the cup 2z linearly back-and-forth, such as laterally side-to-side along the first direction X, as shown in FIG. 7A, or vertically up-and-down or horizontally forward-and-backward along the second direction Y, as shown in FIG. 7B. The translations can occur between various positions P1-Pn along a translation axis, such as linear axis X3 oriented along the first direction X (FIG. 7A) or a linear axis Y3 oriented along the second direction Y (FIG. 7B). In these illustrated examples, the cup 2z begins at a first position P1, which can be substantially centered at the injection site or bleb 11. From the first position P1, the cup 2z can be translated in a first direction (X1 in FIG. 7A; Y1 in FIG. 7B) to a second position P2 on one side of the bleb 11 and then the cup 2z can be translated in a second, opposite direction (X2 in FIG. 7A; Y2 in FIG. 7B) to a third position P3 on the opposite side of the bleb 11. In the illustrated examples, the second and third positions P2, P3 represent the lateral bounds or termini of the translational movements and are spaced from the first position P1 such that the chamber 6 is entirely offset from the bleb 11 at the second and third positions P2, P3. Alternatively, the cup 2z can remain partially over the bleb 11 at the second and/or third position P2, P3, as described below. It should be appreciated that the user can select the first translation direction based on various factors, such as to go with or against the hair grain at the skin surface, by way of a non-limiting example.

As shown, a translation distance T1 between the first position P1 to the second and third positions P2, P3 can be substantially equivalent to the chamber diameter D1, and a translation distance T2 between the second and third positions P2, P3 can be twice (x2) that of translation distance T1. Thus, in the illustrated examples, the cup 2z begins centered on the bleb 11 and translates to side positions P2, P3 that are remote from the bleb 11 (which positions can be referred to as “off-bleb”). For purposes of consistency herein, each translational movement in a consistent direction (e.g., X1, X2, Y1, or Y2) can be referred to as a “pass” or “swipe,” while each such movement between terminal bounds P2, P3 can be referred to as a “full pass” or “full swipe” and each such movement from an intermediate position (e.g., P1) to an end position (e.g., P2, P3) can be referred to a “partial” or “half” pass or swipe.

It should be appreciated that the foregoing translation parameters can be adjusted as needed. For example, the translation distances T1, T2 can vary as needed. Thus, the translation distance T1 need not be equivalent to the chamber diameter D1; for example, the translation distance T1 can be greater than or less than the chamber diameter D1. Additionally or alternatively, the translation distances T1, T2 can be such that the cup 2 remains at least partially over the bleb 11 during translation. Additionally or alternatively, the first position P1 need not be equidistantly spaced from the second and third positions P2, P3. Alternatively, the translational movement can be limited between only first and second positions P1, P2 that are located at the terminal ends of translation (i.e., the first position P1 need not be intermediate the terminal ends). It should also be appreciated that the user can perform various numbers of swipes and/or partial swipes during a kinetic vacuum treatment, and can pause cup movement for various durations between swipes.

Referring now to FIG. 7C, additional example cup movements (kinetics) can include translating the cup 2 along a circuitous path C1 to various positions P1-Pn. The circuitous path C1 can be repeated or partially repeated. Translating the cup 2 along a complete circuitous path C1 can be referred to as a “circuit” and along a portion thereof can be referred to as a “partial circuit.” In the illustrated example, the circuitous path C1 is circular and revolves (i.e., “orbits”) about an axis Z1 that intersects the skin surface along a direction orthogonal thereto. The axis Z1 can intersect the injection site or bleb 11, as shown, or can be offset from the injection site. It should be appreciated, however, that various other translation paths are within the scope of the present disclosure, including eccentric (not centered at axis Z1), elliptical, spiral, triangular or other polygonal, zig-zag, and virtually innumerable others. Additionally, the circuitous translation path C1 can include direction reversals; for example, with reference to the illustrated example, the circuitous path C1 can include translating the cup 2 along a circular orbital path in a first rotational direction R1 (e.g., counter-clockwise) about the axis Z1 and reversing the translation to a second, opposite rotational direction R2 (e.g., clockwise) about the axis Z1. It should be appreciated that numerous variations to the circuitous translation path C1 can be employed as needed during a kinetic vacuum treatment. Additionally or alternatively, during all or part of any of the foregoing orbital movements, the cup 2z can also be tilted relative to an axis that is oriented orthogonally with respect to the underlying tissue so as to cause the cup chamber 6 to keep facing substantially toward the injection site during the orbital movement(s). For example, during an orbit, the cup 2z can optionally be manipulated to undergo a rolling tilt that causes the cup central axis Z1 to substantially remain intersecting a geometric center of the injected bolus 7.

Referring now to FIG. 7D, additional example cup movements (kinetics) can include rotating, pivoting, or “twisting” the cup 2 back-and-forth between various angular positions P1-Pn about an axis Z1 that intersects the skin surface along a direction orthogonal thereto. As shown, the axis Z1 can be centered at the injection site or bleb 11, although it can alternatively be offset therefrom. Each rotational movement between positions can be referred to as a “pivot.” The cup 2 can be pivoted back and forth at various pivot angles A1, A2, A3, An between the angular positions, as needed. In the illustrated example, the cup 2 is shown pivoting from a first position P1 to a second position P2 at a pivot angle A1 of about 90-degrees, from the second position P2 to a third position P3 at a pivot angle A2 of about 180-degrees, and from the third position P3 back to the first position P1 at a pivot angle A3 of about 90-degrees. It should be appreciated, however, that the foregoing are provided as non-limiting examples and that numerous variations to the twisting motions can be employed as needed during a kinetic vacuum treatment.

It should also be appreciated that a user can employ various combinations of the foregoing translational (back-and-forth, circuitous) and rotational (twisting or pivoting) movements during a kinetic vacuum treatment.

Referring again to FIGS. 7A-7D, the various positions of the cup 2z can also be defined by reference to a coordinate system. For example, the linear back-and-forth translations shown in FIGS. 7A and 7B can be defined by reference to a coordinate system, such as a two-dimensional cartesian coordinate system (x,y) having x- and y-axes that extend along the X and Y directions, respectively. In the illustrated examples herein, the x- and y-axes intersect each other at the injection site; thus the coordinate system of these examples is centered at the injection site. Thus, in the examples illustrated in FIGS. 7A and 7B, when the cup 2z is centered over the bleb 11, such as at the first position P1, this cup position can also be denoted as the (0,0) position.

Referring again to FIG. 7A, an exemplary sequence of back-and-forth kinetic movements along the x-axis is further shown in FIG. 7E and can be characterized as follows (a pattern that is also referred to herein as “Pattern A”). From the (0,0) position centered over the bleb 11, the cup 2z can undergo a first translation at a distance “t” in the first direction X1 along the x-axis to the second position P2, which can also be denoted as the (t, 0) position. From the (t, 0) position, the cup 2z can undergo a second translation, at distance t in the second direction X2, back to the (0,0) position, after which the cup 2z can undergo a third translation, at distance t in the second direction X2, to the third position P3, which can also be denoted as the (−t, 0) position. From the (−t, 0) position, the cup 2z can undergo a fourth translation, at distance t along the first direction X1, back to the (0,0) position, which can be the final position in the translation sequence. Thus, the foregoing translation sequence can be characterized as: (0,0)→(t,0)→(0,0)→(−t,0)→(0,0). It should be appreciated that the third translation (0,0)→(−t,0) can occur with no discontinuity or slowing after the second translation (t, 0)→(0,0). In such instances, the second and third translations can be characterized as being two parts of a single translational movement from (t,0)→(−t,0) (i.e., from P2 to P3). It should also be appreciated that the cup 2z can be allowed to dwell at any of the positions (0,0), (t,0), (0,0), (−t,0), (0,0) in the sequence and/or at any intermediate positions therebetween.

Referring again to FIG. 7B, an exemplary sequence of back-and-forth kinetic movements along the y-axis can be characterized as follows. From the (0,0) position, the cup 2z can undergo a first translation, at distance t in a first direction Y1 along the y-axis to, the second position P2, which can also be denoted as the (0,t) position. From the (0,t) position, the cup 2z can undergo a second translation, at distance t in a second direction Y2, back to the (0,0) position, after which the cup 2z can undergo a third translation, at distance t in the second direction Y2, to the third position P3, which can also be denoted as the (0,−t) position. From the (0,−t) position, the cup 2z can undergo a fourth translation, at distance t along the first direction Y1, back to the (0,0) position, which can be the final position in the translation sequence. Thus, the foregoing translation sequence can be characterized as: (0,0)→(0,t)→(0,0)→(0,−t)→(0,0). As above, the third translation (0,0)→(0,−t) can occur with no discontinuity or slowing after the second translation (0,t)→(0,0). In such instances, the second and third translations can be characterized as being two parts of a single translational movement from (0,t)→(0,−t) (i.e., from P2 to P3). It should also be appreciated that the cup 2z can be allowed to dwell at any of the positions (0,0), (0,t), (0,0), (0,−t), (0,0) in the sequence and/or at any intermediate positions therebetween.

The linear back-and-forth translation sequences described above can be repeated any number of times, each of which can be referred to as a “cycle.” It should be appreciated that the sequence of positions can be maintained or reversed from one cycle to the next. Additionally, the cup 2z can be allowed to dwell between cycles or, alternatively, a subsequent cycle can begin immediately after completion of the previous cycle. It should also be appreciated that the linear back-and-forth translation sequences can be adapted in numerous ways without departing from the scope of the disclosed embodiments.

Referring again to FIG. 7C, the revolving (orbital) circuitous path C1 about axis Z1 can also be defined by reference to a coordinate system, such as a two-dimensional polar coordinate system (r,0) centered at axis Z1, where “r” denotes the radial distance from the axis Z1 and “0” denotes the polar coordinate (the angle from the zero-angle position). In the illustrated example, the (0,0) position is coincident with axis Z1. Thus, in the illustrated example, the cup 2z would be at position (0,0) if centered over the bleb 11. Using these polar coordinates, an exemplary sequence of a circular revolving (orbiting) translation will now be described. The cup 2z can be placed at a first position P1 that is offset from axis Z1 by distance t and positioned at the zero-angle polar position, which can be denoted by position (t,0). From this position, the exemplary sequence consists of translating the cup 2z along a circular path C1 that makes one (1) complete revolution (orbit) about axis Z1. Thus, the example revolution includes translating the cup 2z along the circular path C1, in the first rotational direction R1, from the first position P1 (t,0) to a second position P2 (t, π/2), then to a third position P3 (t, x), then to a fourth position P4 (t, 3π/2), and back to the first position P1 (t,0), which can also be denoted as (t, 2π). Thus, the foregoing revolving translation sequence can be characterized as: (t,0)→(t, π/2)→(t, π)→(t, 3π/2)→(t,0). In this example, each of the positions (t,0), (t, π/2), (t, π), (t, 3π/2) are evenly spaced from each other; however in other embodiments, the revolving translation sequence can employ un-evenly spaced angular positions.

Additionally, the revolving translation of this sequence can occur at a substantially constant velocity (i.e., with no discontinuity or slowing between positions), except perhaps with some acceleration at the beginning (from (t,0)) and deceleration at the end of the sequence (to (t,0)). It should also be appreciated that the cup 2z can be allowed to dwell at any of the positions (t,0), (t, π/2), (t, π), (t, 3π/2), (t,0) in the sequence and/or at any intermediate positions therebetween. The sequence can be repeated any number of times, each of which can be referred to as a “cycle.” As discussed above, the rotational direction (and thus the sequence of positions) can be maintained or reversed from one cycle to the next. Additionally, the cup 2z can be allowed to dwell between cycles or, alternatively, a subsequent cycle can begin immediately after completion of the previous cycle. It should be appreciated that the revolving (orbital) translations can be adapted in numerous ways without departing from the scope of the disclosed embodiments.

Referring again to FIG. 7D, the twisting motions of the cup 2z can also be characterized with reference to a polar coordinate system. Thus, the first, second, and third positions P1, P2, P3 of the illustrated example can also be denoted as (0, π/2), (0,0), (0, π), respectively. Similarly as described above, a sequence of twisting motions can be repeated any number of times, each of which can be referred to as a “cycle.” Additionally, the rotational direction (and thus the sequence of positions) can be maintained or reversed from one twist cycle to the next. Moreover, the cup 2z can be allowed to dwell at any position along a cycle and/or between cycles or, alternatively, a subsequent cycle can begin immediately after completion of the previous cycle. It should be appreciated that the twisting motions can be adapted in numerous ways without departing from the scope of the disclosed embodiments.

Referring now to FIG. 7F, an exemplary unidirectional cup translational movement is shown. In this example movement, the cup 2 can be placed at a first position P1 that is offset from a center Z2 of the bleb 11 at a first offset distance along a direction. From the first position P1, the cup can be translated across the bleb center Z2 along the direction to a second location P2, which can be spaced from the bleb center Z2 site at a second offset distance measured along the direction. Preferably, the first and second offset distances are both greater than or equal to (i.e., “no less than”) a maximum interior dimension of the chamber (such as the chamber diameter D1 for circular cup shapes). This offset spacing ensures that the entire vacuum chamber passes over the bleb center Z2 during a unidirectional swipe. However, other offset distances can be employed. When the first and second offset distances are substantially equivalent and both are greater than the maximum interior dimension of the chamber, the unidirectional KV movement across the bleb center Z2 can be referred to herein as “Pattern C”, which can also be denoted as: (−t,0)→(t,0).

Unidirectional kinetic vacuum (KV) movement patterns, such as Pattern C, can be repeated various times (cycles). Between such repetitions, the vacuum field need not be maintained on the tissue. For example, after performing a unidirectional swipe across the bleb, the cup 2 can be removed from the tissue (or the vacuum pressure can be otherwise discontinued) and the cup 2 can be relocated to the first position P1 (or a different position offset from the bleb center Z2), from which position another unidirectional swipe can be performed. It should be appreciated that the foregoing examples represent non-liming examples of a unidirectional translational kinetic vacuum (KV) movement.

In any of the foregoing examples shown in FIGS. 6A-7D, it should be appreciated that the cup can optionally be allowed to lose suction (such as by removing the cup from the tissue) between movements. Stated differently, kinetic vacuum (KV) treatments need not apply active vacuum pressure throughout the entire treatment duration. This can enhance ease of operation for the user, who can elect to remove or “pop” the cup from the tissue and replace it thereon (including at a different position) as needed during a kinetic vacuum (KV) treatment.

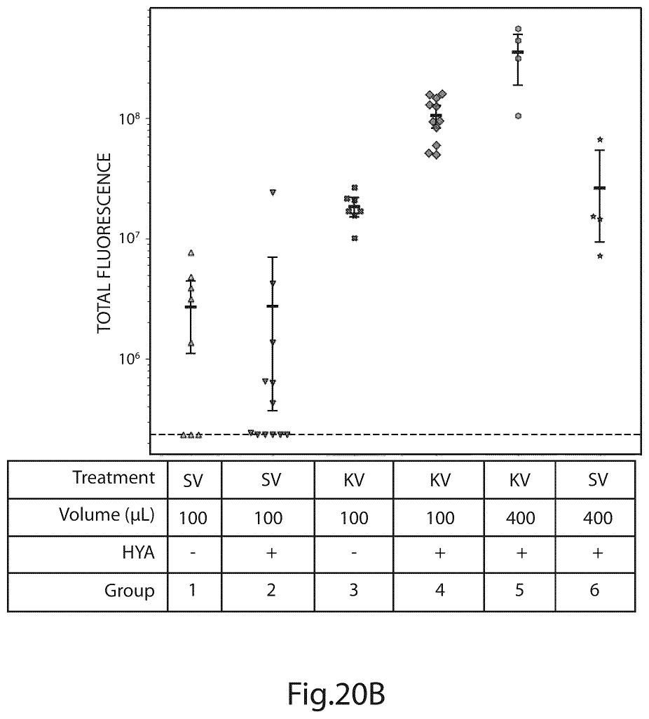

Additionally, although the foregoing examples of kinetic vacuum (KV) treatments illustrate each treatment being administered in connection with a single fluid injection 7, any of the KV treatments herein can be administered to treat multiple fluid injections 7, which injections 7 can be arranged in various patterns, as describe in more detail below with reference to FIG. 24.

It should also be appreciated that any of the foregoing examples of kinetic vacuum (KV) treatments can also employ cup movement in the proximal and/or distal directions P, D (i.e., away from the tissue and/or toward (or into) the tissue, respectively) during, before, or after application of vacuum pressure. Such proximal and/or distal movements can further stress and deform the suctioned tissue to enhance agent delivery.

### Test Results

### Overview

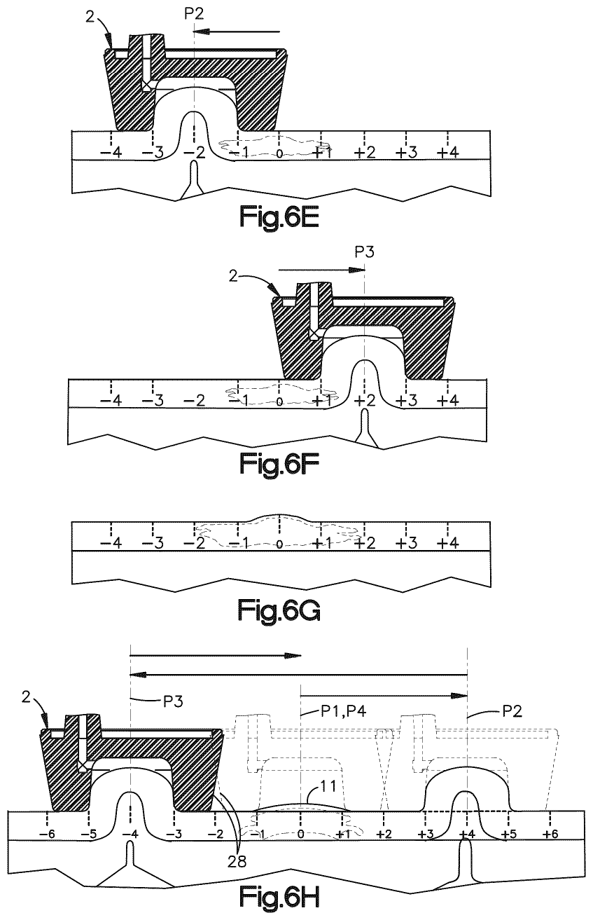

Test results relating to kinetic vacuum treatments are described below with reference to FIGS. 8-46. In these tests, various parameters pertaining to kinetic vacuum (KV) treatments were studied to evaluate, among other things, the efficacy and importance of such parameters, including injection volume and dosing, use of adjuvants, adding hyaluronidase, kinetic vacuum cup movements, cup geometry and diameter, vacuum pressure, and skin thickness at the treatment site, by way of non-limiting examples. Additionally, in these tests, various kinetic vacuum (KV) treatments were studied to evaluate their performance (e.g., gene expression, immune response, potential damage to skin surface) in comparison to other vaccine-related treatment techniques, including vacuum electroporation (VEP), static vacuum (SV) treatments, injection-only (INJ) treatments, needle electroporation (NEP), intramuscular electroporation (IM-EP), and mRNA vaccine injections. It should be appreciated that in the following description of the studies, all plasmid injections were performed via Mantoux injection into intradermal (ID) tissue (i.e., layers of the skin), unless stated otherwise (such as for intramuscular electroporation (IM-EP) treatments and intramuscular (IM) mRNA injections).

The following treatments and their test results include the following:

### Injection Only (INJ):

An intradermal injection. In subject guinea pigs, the injection is performed in the skin over the flank. In subject rabbits, the injection is typically performed in the skin over the quadricep. The injection only (INJ) treatments discussed below did not involve vacuum treatment or electroporation.

### Static Vacuum (SV):

An intradermal injection followed by the application of negative pressure (vacuum pressure) via a vacuum cup. The vacuum pressure is used to evacuate the vacuum cup and pull the targeted tissue (e.g., skin) into the cup. The skin is then acted upon by the vacuum pressure for a predetermined amount of time until the vacuum pressure ceases. After the pressure inside the vacuum cup returns to normal, the cup is removed from the skin. The static vacuum (SV) treatments discussed below did not involve kinetic vacuum movements or electroporation. Unless stated otherwise below, each static vacuum (SV) treatment applied vacuum pressure for a predetermined amount of time of about 10-20 seconds (which duration extends from vacuum start-up to shut off). This 10-20-second vacuum period was selected for correlation to certain VEP durations (including vacuum start, EP pulsing, and vacuum shut off).

### Kinetic Vacuum (KV):

An intradermal injection followed by the application of vacuum pressure via a vacuum cup. The vacuum pressure is used to evacuate the vacuum cup and pull the targeted tissue (e.g., skin) into the cup. After the skin has been pulled into the cup, the vacuum cup is manipulated by an external force causing the vacuum cup to move (i.e., translate, rotate, or a combination thereof) relative to the skin. After the treatment is completed, the vacuum pressure ceases and the external force is removed. Once the pressure inside the cup returns to normal, the vacuum cup is removed. The kinetic vacuum (KV) treatments discussed below did not involve electroporation. Additionally, unless stated otherwise below, the KV treatments evaluated below employed the following linear side-to-side sequence (also referred to below as “Pattern A”): (0,0)→(t,0)→(0,0)→(−t,0)→(0,0), which sequence was performed three (3) times (i.e., 3 cycles) per treatment. Also, unless stated otherwise below, vacuum pressure was applied at approximately −500 mmHg. Moreover, unless stated otherwise below, each tested kinetic vacuum (KV) treatment below resulted in vacuum pressure being applied for about 10-20 seconds (which duration includes vacuum start-up, reaching the desired vacuum pressure, followed by cup translation(s) and vacuum shut-off). As above, this 10-20-second vacuum period was selected for correlation to certain VEP durations (including vacuum start, EP pulsing, and vacuum shut off).

### Vacuum Electroporation (VEP):

An intradermal injection followed by non-invasive electroporation targeting the dermis and epidermis using a vacuum cup with at least one electrode within the chamber acting on the surface of the skin. Vacuum pressure is used to evacuate the vacuum cup to pull targeted tissue (e.g., skin) into contact with the electrode(s). All VEP test results discussed below were generated using the centerpost-electrode VEP cup 402 shown in FIGS. 4A-4B. Unless stated otherwise below, the electroporation component of the VEP treatments below involved three consecutive electroporative pulses applied, each pulse having a pulse duration of 50 milliseconds (ms), an electric current of 0.5 Amp, a maximum voltage of 200 Volts (V), with an interpulse delay of 250 ms between pulses. For each pulse, the ring electrode acts as the delivery electrode and the post electrode acts as the return electrode. Unless stated otherwise, the total duration of each VEP treatment was about 10-20 seconds (including vacuum start, EP pulsing, and vacuum shut off.). The vacuum electroporation (VEP) treatments discussed below did not involve kinetic vacuum movements.

### Needle Electroporation (NEP):

An intradermal injection followed by invasive electroporation using three (3) needle electrodes each having an outer diameter of 0.46 mm. The three (3) needle electrodes are arranged in an isosceles triangle, where one pair of the electrodes (i.e., the first and second electrodes) are spaced 3 mm apart from each other, while the third electrode is spaced from each of the first and second electrodes by 5 mm. The three (3) needle electrodes are inserted into the dermis and epidermis at a depth of 3 mm from the skin surface. Four (4) consecutive electroporative pulses are applied, each pulse having a pulse duration of 52 ms, an electric current of 0.2 Amp, a maximum voltage of 200 V, with an interpulse delay of 250 ms between pulses. During pulses 1 and 3, the first electrode acts as the source electrode while the second and third electrodes act as return electrodes. During pulses 2 and 4, the second electrode delivers the pulse and electrode 3 acts as a return electrode. The needle electroporation (NEP) treatments discussed below did not involve vacuum treatment (neither KV nor SV) or vacuum electroporation (VEP).

### Intramuscular Electroporation (IM-EP):

An intramuscular injection of pDNA at a targeted depth in quadriceps muscle using a bolus needle, followed by invasive electroporation using 5P array through the skin and into quadriceps muscle. The 5P array has five (5) needle electrodes arranged in an equilateral pentagon pattern having a pattern diameter of 10 mm. The five (5) needle electrodes are configured for an insertion depth of about 19 mm. Three (3) consecutive electroporative pulses are applied, each pulse having a pulse duration of 52 ms, an electric current of 0.5 Amp, a maximum voltage of 200 V, with an interpulse delay of 1 second between pulses. The three (3) electroporative pulses are delivered by the electrodes as follows:
Pulse 1: from electrode 1 (positive) to electrodes 3 and 4 (negative);
Pulse 2: from electrode 2 (positive) to electrodes 4 and 5 (negative); and
Pulse 3: from electrode 3 (positive) to electrode 5 (negative).

The intramuscular electroporation (IM-EP) treatments discussed below did not involve vacuum treatment (neither KV nor SV) or vacuum electroporation (VEP).

### Additional Test Details:

Some of the tests below involved Off-the-Shelf (OTS) vacuum devices. Details regarding these OTS devices are shown in Table 0 below:

TABLE 0

| | | Cup Size | Measured |
|---|---|---|---|
| Device | | (Chamber Dia.) | Vacuum Pressure |
| Label | Cup Shape | (mm) | (mmHg) |
| cup 102 | Circle | 12 | −500 |
| OTS-0 | Circle | 14 | −420 |
| OTS-1 | Circle | 10 | −508 |
| OTS-2 | Circle | 9.5 | −474 |
| OTS-3 | Rounded Rectangle | 7.75 × 6.33 | −474 |
| OTS-4 | Interchangeable | Interchangeable | −490 |
| | (e.g., Circle, Ellipse) | (e.g., 3.0-11.75) | |
| OTS-5 | Circle | 9.9 | −492 |
| OTS-6 | Circle | 6 | 489 |

### Study 1

Referring now to FIG. 8, gene expression in guinea pig skin is shown after intradermal injections of uniform volumes of a plasmid encoding the gene for green fluorescent protein (GFP) and then treatment according to four (4) treatment groups:
Group 1: VEP treatment using a vacuum cup having a chamber diameter D1 of 12 mm;
Group 2: SV treatment using a centerpost vacuum cup 102 (see FIGS. 2A-2C) having a chamber diameter D1 of 12 mm;
Group 3: SV treatment using a dome vacuum cup 2 (see FIGS. 1A-1D) having a chamber diameter D1 of 12 mm; and
Group 4: KV treatment, using the same dome vacuum cup design in Group 3.

Each Group involved two (2) samples (1a,b; 2a,b; 3a,b; 4a,b), each sample receiving three (3) injections aligned in a column in the skin over the flank. Each injection contained plasmid 5013 (which encodes the gene for GFP) at a volume of 100 uL with a DNA dose of 0.5 ug. Further details regarding the test parameters for this study are shown in Table 1 below:

TABLE 1

| Group Number (n/group) | Plasmid | Treatment | Center Post |
|---|---|---|---|
| 1. (n = 2) | 5013 | VEP | + |
| 2. (n = 2) | 5013 | SV | + |
| 3. (n = 2) | 5013 | SV | − |
| 4. (n = 2) | 5013 | KV | − |

In this study, SV treatments—both with the centerpost cup (2a,b) and dome cup (3a,b)—produced a detectable GFP signal, with the dome cup (3a,b) having somewhat of a donut-shaped signal with a void in the central area of the injection site, and the centerpost cup (2a,b) eliminating the central void (essentially “filling in” the donut, so to speak). In this study, KV treatments using the dome cup (4a,b) produced a comparable GFP signal to the static vacuum treatments (2a,b) but with larger signal areas. Regarding the VEP treatments (1a,b), the GFP signal appears lower than the other Groups, which likely results from the fact that tissue damage caused by electroporation has been shown to hide GFP signal.

### Study 2

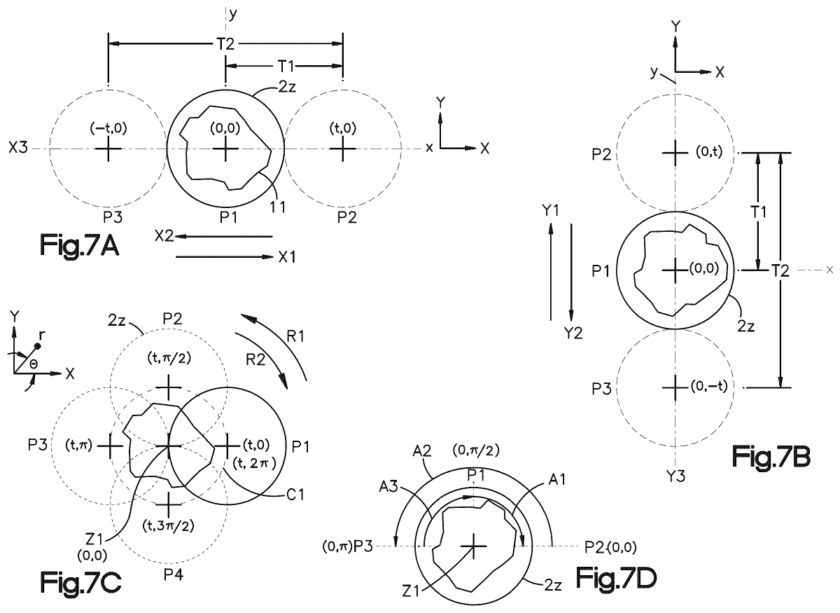

Referring now to FIG. 9, GFP gene expression in guinea pig skin is shown after treatments involving various kinetic vacuum cup movements applied by various vacuum cup designs. Subjects were injected intradermally with uniform volumes of a plasmid encoding the gene for GFP and were then treated according to seven (7) vacuum treatment groups:
Group 1: KV treatment using side-to-side (STS) movement (Pattern A, 3 cycles) (see FIG. 7A), performed by a centerpost vacuum cup 102 (see FIGS. 2A-2C) having a chamber diameter D1 of 12 mm;
Group 2: KV treatment using up-and-down (UD) movement (see FIG. 7B) performed by the same centerpost vacuum cup 102 design in Group 1;
Group 3: KV treatment using the same STS movement as Group 1 but performed by a dome vacuum cup 2 (see FIGS. 1A-1D) having a chamber diameter D1 of 12 mm;
Group 4: kinetic treatment without vacuum (K, no vac) using the same STS movement and cup 102 design as Group 1;
Group 5: KV treatment using the same STS movement as Group 1 but performed by a dome vacuum cup 2 having a chamber diameter D1 of 15 mm;
Group 6: KV treatment using a circuitous orbital (ORB) movement (see FIG. 7C), particularly by pulling the cup along a circular path around the injection site in two (2) orbits/cycles, counterclockwise and then clockwise (i.e., the first circular orbit in a counterclockwise rotational direction R1, the second along the same circular orbit but in a clockwise rotational direction R2, both cycles performed without twisting or pivoting motion about central axis Z1), performed by the same centerpost vacuum cup 102 design in Group 1; during the particular orbital movements employed for this Group, the cup 102 also effectively underwent a rolling vertical tilt that caused the cup central axis Z1 to substantially remain intersecting a geometric center of the bleb 11;
Group 7: KV treatment using a twisting (TWT) movement (see FIG. 7D), particularly by twisting the cup 360-degrees clockwise about the central axis Z1, then twisting the cup 360-degrees counterclockwise about the central axis Z1), performed by the same centerpost vacuum cup 102 design in Group 1.

Groups 1-5 involved two (2) samples (“a” and “b”) each, while Groups 6-7 involved one (1) sample (“a”) each, wherein each of the foregoing samples in this study received three (3) ID injections on the flank, shown in generally columnar fashion. Each injection site received an independent vacuum treatment. Accordingly, Groups 1-5 each have six (6) replicates spread across two (2) flanks, while Groups 6-7 each have three (3) replicates spread across one (1) flank. Each injection contained plasmid 5013 (which encodes the gene for GFP) at a volume of 100 uL with a DNA dose of 0.5 ug. For Groups 1-3 and 5-7, vacuum pressure for each treatment was applied for 15 seconds to correlate with VEP durations. Group 4 was the only treatment in this study that did not employ vacuum pressure. Further details regarding the test parameters for this study are shown in Table 2 below:

TABLE 2

| Group Number | | | Center- | |
|---|---|---|---|---|
| (n/group) | Plasmid | Treatment | Post | Movement Pattern |
| 1. (n = 2) | 5013 | KV | + | STS: (0, 0)→(t, 0)→(0, 0)→(−t, 0)→(0, 0), |
| | | | | Repeated 3 times |
| 2. (n = 2) | 5013 | KV | + | UD: (0, 0)→(0, t)→(0, 0)→(0, −t)→(0, 0), |
| | | | | Repeated 3 times |
| 3. (n = 2) | 5013 | KV | − | STS: (0, 0)→(t, 0)→(0, 0)→(−t, 0)→(0, 0), |
| | | | | Repeated 3 times |
| 4. (n = 2) | 5013 | K, No Vac | + | STS: (0, 0)→(t, 0)→(0, 0)→(−t, 0)→(0, 0), |
| | | | | Repeated 3 times |
| 5. (n = 2) | 5013 | KV, 15 mm | − | STS: (0, 0)→(t, 0)→(0, 0)→(−t, 0)→(0, 0), |
| | | | | Repeated 3 times |
| 6. (n = 2) | 5013 | KV | + | ORB: (0, 0) Orbited 360 degrees, |
| | | | | Repeated 3 times. |
| 7. (n = 2) | 5013 | KV | + | TWT: (0, 0), rotated 360 degrees, |
| | | | | Repeated 3 times |

From this study, it can be seen that KV treatments (Groups 1-3 and 5-7; images 1a,b, 2a,b, 3a,b, 5a,b, 6a,b, 7a,b) produce similar GFP responses, while kinetic treatment without vacuum (Group 4; images 4a,b) produced a weaker signal (GFP response) than the KV treatments. This study also demonstrates that, for vacuum cups having the same size, the centerpost cups 102 and the dome cups 2 produced similar GFP responses (compare results for the centerpost vacuum cups in Groups 1-2 and 6-7 with the dome cup results of Group 2, each cup having a chamber diameter D1 of 12 mm). This study further suggests that increasing the cup size (diameter) might reduce the GFP signal in some instances (compare Group 5 results 5a,b (dome cup 2 with D1=15 mm) with Group 3 results 3a,b (dome cup 2 with D1=12 mm), both groups 5 and 3 moving the cup side-to-side), although more testing would be necessary regarding the standalone effect of cup size/diameter on GFP signal. This study yet further shows that the circular orbital cup movement (Group 6) and twisting cup movement (Group 7) produced less of a GFP response than the linear translation cup movements (Groups 1-5).

### Study 3

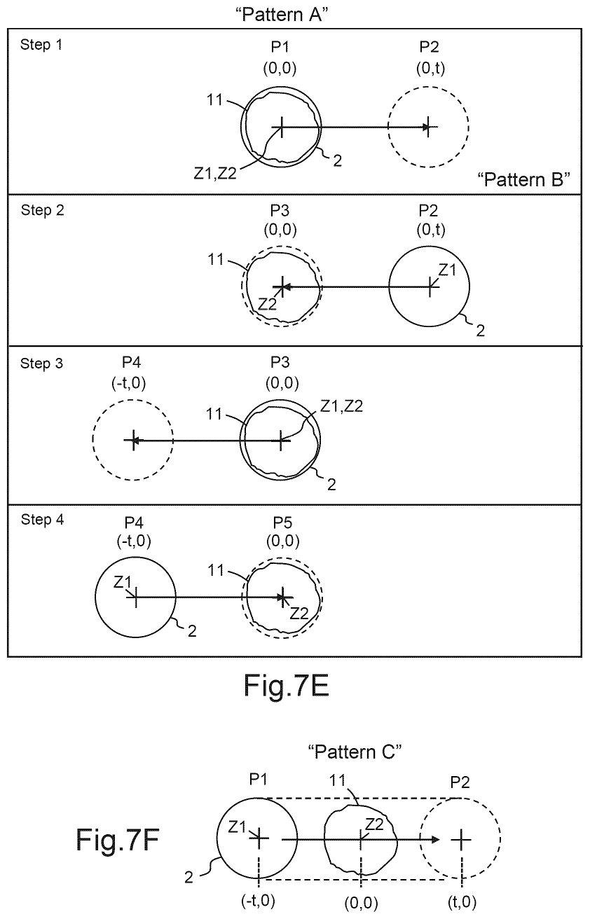

Referring now to FIGS. 10A and 10B, immune responses (ELISA expression) in guinea pigs was tested to evaluate immune responses following kinetic vacuum (KV) treatments in comparison to immune responses following various static vacuum (SV) treatments, vacuum electroporation (VEP) treatments, and injection-only (INJ) treatments.

Subjects were injected intradermally with uniform volumes of a plasmid and were then treated according to the following treatment groups:
Group 1: VEP treatment using a centerpost-electrode VEP cup 402 (see FIGS. 4A-4B);
Group 2: SV treatment using the same cup design from Group 1;
Group 3: SV treatment using a dome vacuum cup 2 (see FIGS. 1A-1D);
Group 4: KV treatment using the same centerpost cup design from Group 1; and
Group 5: INJ treatment.

Each Group involved six (6) samples, each sample receiving one (1) ID injection on the flank. Each injection contained plasmid 2027, which encodes an influenza antigen, at a volume of 100 uL with a DNA dose of 0.05 ug. Group 4 was the only treatment in this study that employed KV. Further details regarding the test parameters for this study are shown in Table 3 below:

TABLE 3

| Group Number | | | |
|---|---|---|---|
| (n/group) | Plasmid | Delivery | Center Post |
| 1. (n = 6) | 2027 | VEP | + |
| 2. (n = 6) | 2027 | SV | + |
| 3. (n = 6) | 2027 | SV | − |
| 4. (n = 6) | 2027 | KV | + |
| 5. (n = 6) | 2027 | INJ | N/A |

Combined binding titer data for all Groups is shown at Week 2 (FIG. 10A) and Week 4 (following second vaccination) (FIG. 10B). In the results of this study, the KV treatment (Group 4) produced an immunogenicity that was comparable to VEP treatment (Group 1) and superior to SV treatments (Groups 2 and 3). Among the SV treatments, the centerpost vacuum cup (Group 2) produced superior immunogenicity than that produced by the dome vacuum cup (Group 3). The inventors found it surprising and unexpected that the KV treatments (Group 4) produced an immunogenicity that was substantially equivalent to that produced by the VEP treatments (Group 1). The ability to use KV treatments without electroporation to produce immune responses substantially equivalent to those produced using electroporation (with vacuum or other techniques) could be revolutionary in the field of vaccine administration. For example, the inventors believe that the KV treatments disclosed herein (without electroporation) provide significant improvements to both the instantaneous pain/irritation and the healing period associated with electroporation treatments (particularly invasive electroporation treatments) and, in a more general sense, to those associated with viral vector-based, mRNA based, protein-based, nanoparticle based, and other methods which may benefit from the pain-free, localized agent delivery provided by KV treatments. The KV treatments disclosed herein are also believed to have the potential to be beneficial in medical fields beyond agent delivery.

Referring now to FIGS. 10C-10D, the visible effects on the skin imparted by the various treatments discussed above with reference to FIGS. 10A-10B are shown. In particular, FIG. 10C shows the treatment sites immediately after the applied treatment; and FIG. 10D shows the treatment sites at seven (7) days post-treatment. In these Figures, the images showing the treatment sites of the various Groups are labeled as follows: Group 1-275L-280L; Group 2-281L-286L; Group 3-287L-292L; Group 4-293L-298L; and Group 5-299L-304L. It can be seen that the KV treatment (Group 4, images 293L-298L) caused acute redness and irritation on the skin surface immediately following treatment (FIG. 10C), although by Day 7 there was no visible tissue damage (FIG. 10D). By comparison, the VEP treatment did not cause immediate acute visible tissue damage (FIG. 10C), but by Day 7 some scabbing and/or other superficial tissue damage was observed (FIG. 10D).

### Study 4

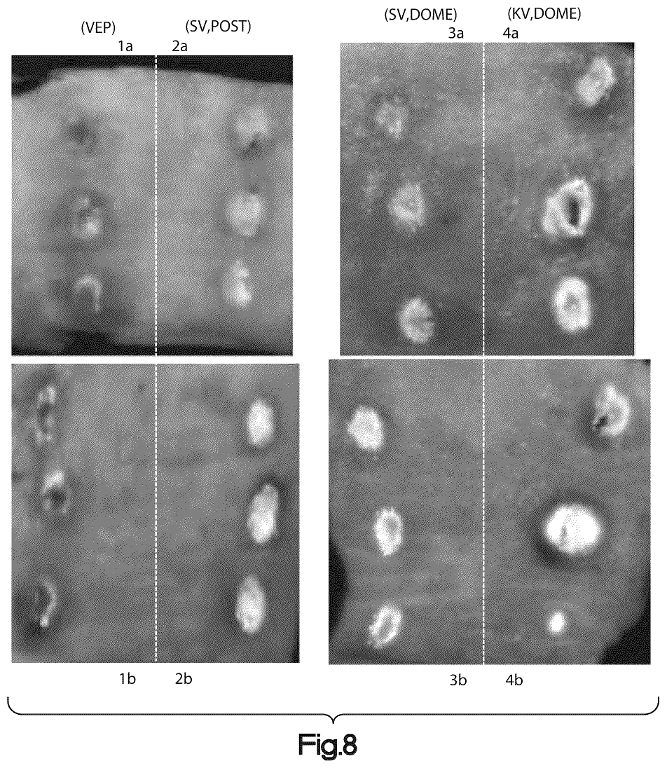

Referring now to FIGS. 11A and 11B, immune responses (ELISA expression) in guinea pigs were tested again to effectively replicate the study shown in FIGS. 10A-10B to confirm the ELISA results thereof. The same five (5) treatment groups were tested, with the main difference being in the plasmid used (plasmid 2303 in the present study) and the DNA dose (0.6 ug in the present study). Further details regarding the test parameters for this study are shown in Table 4 below:

TABLE 4

| Group Number | | | Center | DNA Dose |
|---|---|---|---|---|
| (n/group) | Plasmid | Delivery | Post | (ug) |
| 1. (n = 6) | 2303 | VEP | + | 60 |
| 2. (n = 6) | 2303 | SV | + | 60 |
| 3. (n = 6) | 2303 | SV | − | 60 |
| 4. (n = 6) | 2303 | KV | + | 60 |
| 5. (n = 6) | 2303 | INJ | N/A | 60 |

The ELISA data for Groups 1-5 are shown in FIGS. 11A and 11B. In particular, FIG. 11A shows combined titer data for all Groups at Week 2 and FIG. 11B shows combined titer data for all Groups at Week 4 (following second vaccination). As shown, as in the study shown in FIGS. 10A-10B, the KV treatments (Group 4) produced an immunogenicity that was comparable to VEP treatment (Group 1). In the present study, however, the KV treatment (Group 4) did not meaningfully outperform the SV treatments (Groups 2 and 3) at Week 4. The KV treatments (Group 4) did show increased ELISA response at Week 2 relative to the SV treatments (Groups 2 and 3), but showed similar responses at Week 4 after the second vaccination had been administered. Moreover, at Week 2, the KV treatments (Group 4) showed comparable ELISA responses to those of the VEP treatments (Group 1). Also shown in the present study, the INJ treatment (Group 5) demonstrated strong ELISA responses at Weeks 2 and 4. Typically, seroconversion is not observed at Week 2 for injection-only treatments like those shown in the present study. These unusually strong results for the INJ treatments (Group 5) might make it harder to differentiate the best and worst ELISA responses in this particular study considering that the low-end baseline is unusually high. Though wishing not to be bound by any particular theory, the inventors believe that the unusually strong results for the INJ treatments suggest that that the DNA dosages in this study (0.6 ug for each injection) were higher than necessary for this particular animal (guinea pig), which is supported by the Week 4 ELISA responses, which continue to overlap across all Groups, with even the weakest responses being substantially higher than the detection limit.

### Study 5

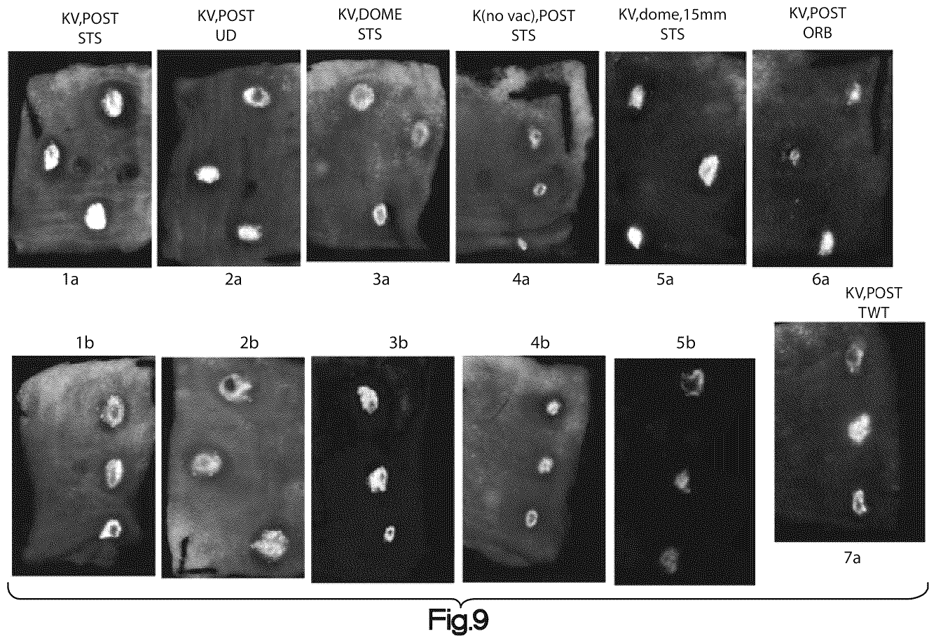

Referring now to FIGS. 12A and 12B, a study attempted to explore the effect of reducing DNA doses (via reduction in injection volume) with kinetic vacuum (KV) treatment on immune responses (binding ELISA responses) in guinea pigs. In this study, subjects in two groups (Group 1 and Group 2) were injected intradermally with a plasmid and then treated via KV using the same centerpost cup design. The only difference between the Groups was the agent volume: Group 1 administered an injection volume of 100 uL, while Group 2 administered a reduced injection volume of 50 μL. The agent in both Groups had a DNA concentration of 0.05 mg/mL. Both Groups involved five (5) samples, each sample receiving one (1) ID injection of plasmid 2027 on the right flank. The ELISA responses for both Groups at Week 2 are shown in FIG. 12A and at Week 4 are shown in FIG. 12B.

As indicated, the reduced dose (Group 2) had no detectable impact on ELISA response at Week 2 (FIG. 12A), but showed a mild reduction in titers at Week 4 (after the second vaccine dose) (FIG. 12B). Based on this study, it is not entirely clear whether KV is sensitive to half-dose since the difference between groups was small and non-significant, but this study provides some evidence that the immunogenicity of KV treatments may scale with injection volume.

### Study 6

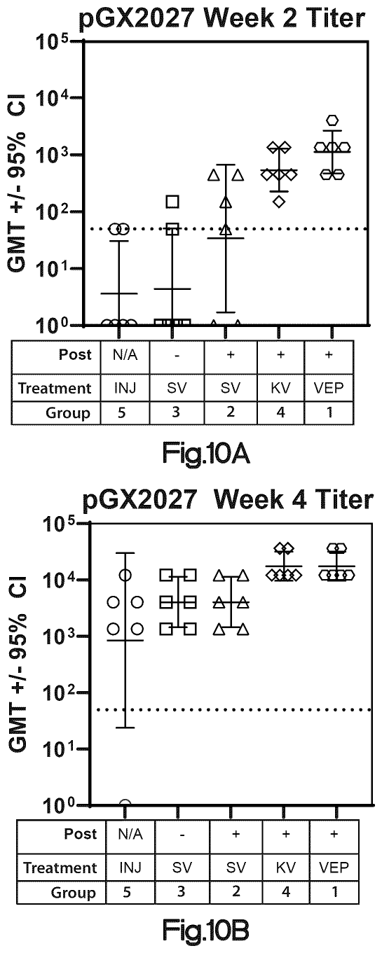

Referring now to FIG. 13, immune responses (ELISA expression) in naïve guinea pigs was tested to evaluate immune responses following kinetic vacuum (KV) treatments in comparison to immune responses following vacuum electroporation (VEP) treatments, static vacuum (SV) treatments, and injection-only treatments.

Subjects were injected intradermally with uniform volumes of a plasmid and were then treated according to the following treatment groups:
Group 1: VEP treatment using a centerpost-electrode VEP cup 402 (see FIGS. 4A-4B);
Group 2: KV treatment using a centerpost vacuum cup 102 design (see FIGS. 2A-2C);
Group 3: SV treatment using the same centerpost vacuum cup 102 design used in Group 2;
Group 4: INJ treatment.

Each Group involved eight (8) samples, each sample receiving one (1) ID injection in the left flank. Each injection contained plasmid 2027 at a volume of 100 uL with a DNA dose of 0.05 ug. Further details regarding the test parameters for this study are shown in Table 5 below:

TABLE 5

| Group Number | | |
|---|---|---|
| (n/group) | Plasmid | Delivery |
| 1. (n = 8) | 2027 | VEP |
| 2. (n = 8) | 2027 | KV |
| 3. (n = 8) | 2027 | SV |
| 4. (n = 8) | 2027 | INJ |

Titer data is shown for Groups 1-4 at Week 2. The results of this study are consistent with the studies above in non-naïve subjects, showing that ELISA responses produced by SV treatments are greater than those produced by INJ treatments, and that KV treatments produce ELISA responses that are greater than those produced by SV and INJ treatments and that are on par with those produced by VEP treatments. One item of note from this study: one subject in Group 2 was removed from the study because anesthesia was interrupted during vaccination, preventing the vacuum procedure from being properly applied after the injection. The removal of this subject does not affect the overall results described above.

### Study 7

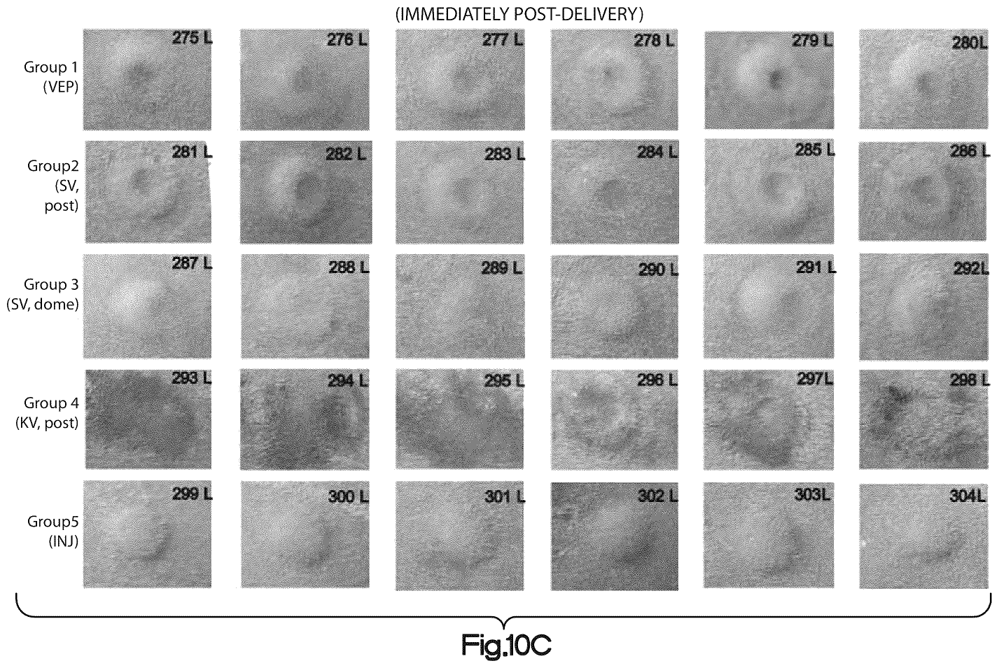

Referring now to FIG. 14, this study evaluates the effects that the number of translations or “swipes” and the usage of hyaluronidase has on immune response (ELISA expression) in guinea pigs that received kinetic vacuum (KV) treatments.

Subjects were injected intradermally with uniform volumes of a plasmid and then treated according to the following treatment groups:
Group 1: KV treatment (Pattern A, 3 cycles-see FIG. 7A);
Group 2: KV treatment employing one single linear translation (one swipe), (0,0)→(t,0), also referred to herein as “Pattern B”, 1 cycle, using the same centerpost vacuum cup design from Group 1; and
Group 3: KV treatment the same as Group 1 (Pattern A, 3 cycles) but using a dome vacuum cup 2 (see FIGS. 1A-1D);
Group 4: KV treatment, the same as Group 2 (Pattern B, 1 cycle) but using the same dome vacuum cup design from Group 3; and
Group 5: KV treatment, the same as Group 1 (Pattern A, 3 cycles), but with the injection including hyaluronidase.

Each Group involved six (6) samples, each sample receiving one (1) intradermal (ID) injection on the left flank. Each injection contained plasmid 9517 at a volume of 100 uL with a DNA concentration of 0.25 mg/mL. As mentioned above, the injection for Group 5 included hyaluronidase. Combined titer results are shown in FIG. 14. Further details regarding the test parameters for this study are shown in Table 6 below:

TABLE 6

| Group | | | | | | |
|---|---|---|---|---|---|---|
| Number | | Treat- | Center | Movement | # | |
| (n/group) | Plasmid | ment | Post | Cycle Pattern | Cycles | HYA |
| 1. (n = 6) | 9517 | KV | + | Pattern A: | 3 | − |
| | | | | (0, 0)→(t, 0)→ | | |
| | | | | (0, 0)→(−t, 0)→ | | |
| | | | | (0, 0) | | |
| 2. (n = 6) | 9517 | KV | + | Pattern B: | 1 | − |
| | | | | (0, 0)→(t, 0) | | |
| 3. (n = 6) | 9517 | KV | − | Pattern A: | 3 | − |
| | | | | (0, 0)→(t, 0)→ | | |
| | | | | (0, 0)→(−t, 0)→ | | |
| | | | | (0, 0) | | |
| 4. (n = 6) | 9517 | KV | − | Pattern B: | 1 | − |
| | | | | (0, 0)→(t, 0) | | |
| 5. (n = 6) | 9517 | KV | − | Pattern A: | 3 | + |
| | | | | (0, 0)→(t, 0)→ | | |
| | | | | (0, 0)→(−t, 0)→ | | |
| | | | | (0, 0) | | |

In the results, Groups 1, 3, and 5, which employed Pattern A, 3 cycles (6 total swipes), produced greater immune response than the Pattern B, 1 cycle (1 swipe) groups (Groups 2 and 4). The results also demonstrate that the addition of hyaluronidase to a KV treatment significantly improves immunogenicity. Additionally, by comparing the results of Group 1 with Group 2 and comparing Group 3 with Group 4, it is unclear from this study whether the centerpost vacuum cup 102 meaningfully improves immunogenicity over the dome cup 2 for KV treatments, although the data trends slightly that way.

### Study 8

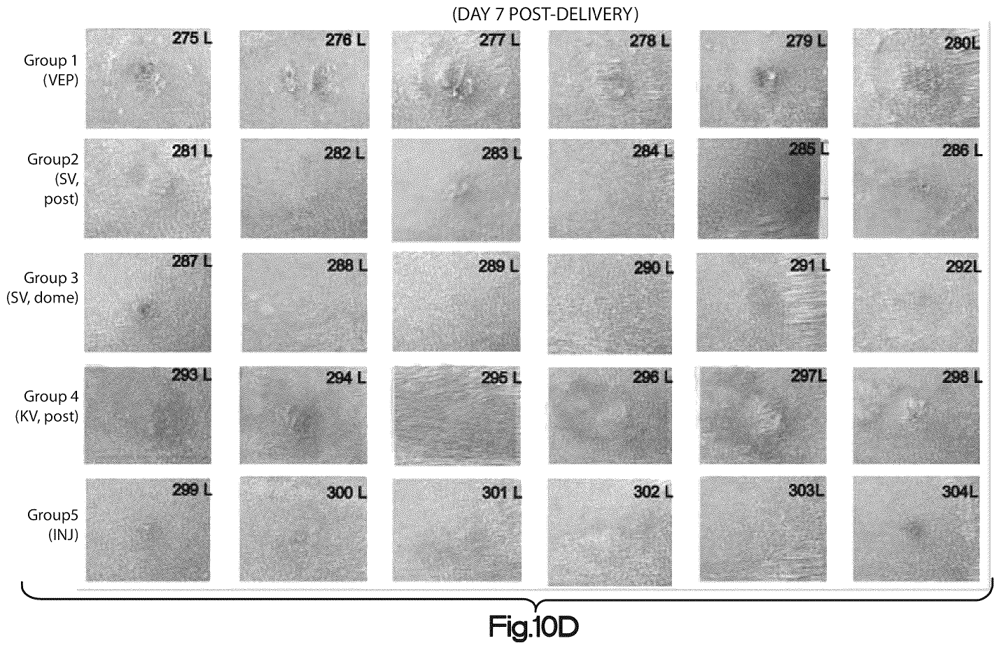

Referring now to FIG. 15, this study evaluates the effects that vacuum pressure has on immune response (ELISA expression) in guinea pigs that received otherwise similar kinetic vacuum (KV) treatments.

Subjects in five (5) groups were injected intradermally with uniform volumes of a plasmid and then administered KV treatments that employed different vacuum pressures but the same kinetic vacuum cup movements (Pattern A, 3 cycles). The groups employed the following vacuum pressures: Group 1 used −100 mmHG; Group 2 used −300 mmHG; Group 3 used −500 mgHg; Group 4 used −600 mmHg (the maximum vacuum pressure of the pump used for this study); and Group 5 used about −420 mmHg. Groups 1-4 performed the KV treatments using the same centerpost cup 102 design. Group 5 used an off-the-shelf (OTS) vacuum applicator, which is labeled “OTS-0” in Table 0, which is the MarvelouSlim applicator, produced by Zemits Kosmetik Experte, headquartered in Carlsbad, California, United States. The OTS-0 applicator uses a vacuum head having a chamber diameter of about 14 mm and having distal rollers (ball bearings seated in the distal surface) that facilitates translation of the vacuum head across the skin surface.

Each Group involved six (6) samples, each sample receiving one (1) intradermal (ID) injection on the left flank. Each injection contained plasmid 2303 at a volume of 100 uL with a DNA concentration of 0.3 mg/mL. For the kinetic vacuum cup movements, each Group employed three (3) sets of linear translations (six (6) total swipes) across the injection site. Further details regarding the test parameters for this study are shown in Table 7 below:

TABLE 7

| Group Number | | | Vacuum | Vacuum Pressure |
|---|---|---|---|---|
| (n/group) | Plasmid | Treatment | Device | (mmHg) |
| 1. (n = 6) | 2303 | KV | cup 102 | −100 |
| 2. (n = 6) | 2303 | KV | cup 102 | −300 |
| 3. (n = 6) | 2303 | KV | cup 102 | −500 |
| 4. (n = 6) | 2303 | KV | cup 102 | −600+ |
| 5. (n = 6) | 2303 | KV | OTS-0 | ~−420 |

Combined ELISA results for each Group are shown in FIG. 15. These results show a clear correlation between vacuum strength and immunogenicity. In particular, ELISA responses increased with increasing vacuum pressure. Additionally, in this study the OTS-0 applicator at about −420 mmHg (Group 5) produced generally comparable results to the −500 and −600 mmHg centerpost cups (Groups 3 and 4), although the inventors observed that the OTS-0 applicator tended to detach inadvertently from the skin surface during translation (swipes). That the OTS-0 applicator detached from the skin surface during use was not surprising, considering that the OTS-0 applicator literature mentions the ability to detach for ease of comfort during use. The inventors did find it surprising, however, that the OTS-0 applicator's detachment during use did not appear to meaningfully diminish the immunogenicity results.

### Study 9

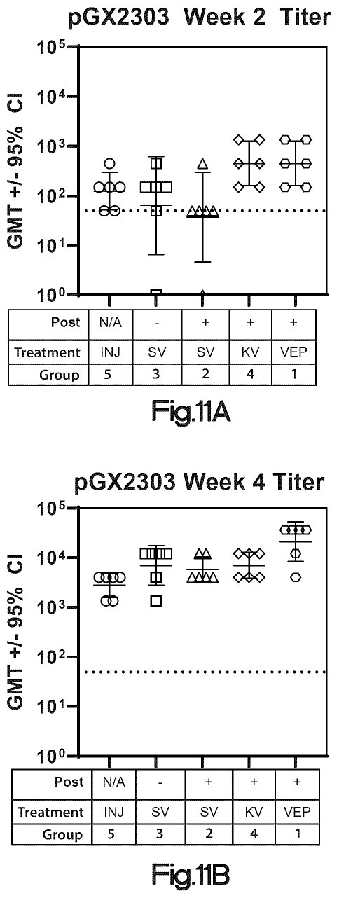

Referring now to FIGS. 16A-16B, this study evaluates immune responses (ELISA expression) in rabbits, particularly comparing the immune responses produced by kinetic vacuum (KV) treatments relative to the immune responses produced by treatments involving injection-only (INJ), static vacuum (SV), vacuum electroporation (VEP), and needle electroporation (NEP).

Subjects were injected intradermally with uniform volumes of a plasmid and were then treated according to the following treatment groups:
Group 1: INJ treatment;
Group 2: SV treatment using a centerpost vacuum cup 102 design (see FIGS. 2A-2C) having a chamber diameter D1 of 12 mm;
Group 3: KV treatment using the same centerpost vacuum cup 102 design used in Group 2;
Group 4: VEP treatment using a centerpost-electrode VEP cup 402 (see FIGS. 4A-4B); and
Group 5: NEP using the CELLECTRA 2000-3P device, produced by Inovio Pharmaceuticals, Inc., headquartered in Plymouth Meeting, Pennsylvania, United States.

Each Group involved five (5) samples, each sample receiving one (1) ID injection in the skin over the left quadriceps. Each injection contained plasmid 9517 at a volume of 100 uL with a DNA dose of 2.5 mg/mL. Further details regarding the test parameters for this study are shown in Table 8 below:

TABLE 8

| Group Number | | |
|---|---|---|
| (n/group) | Plasmid | Treatment |
| 1. (n = 5) | 9517 | INJ |
| 2. (n = 5) | 9517 | SV |
| 3. (n = 5) | 9517 | KV |
| 4. (n = 5) | 9517 | VEP |
| 5. (n = 5) | 9517 | NEP |

Titer data for all groups is shown at Day 0 (FIG. 16A) and Week 2 (FIG. 16B). The results of this study are consistent with the studies in guinea pigs described above, showing that ELISA responses produced by KV treatments (Group 3) are greater than those produced by SV treatments (Group 2), which are greater than those produced by INJ treatments (Group 1). Surprisingly and unexpectedly, in this study KV treatments produced greater ELISA responses than both VEP treatments (Group 4) and NEP treatments (Group 5).

### Study 10

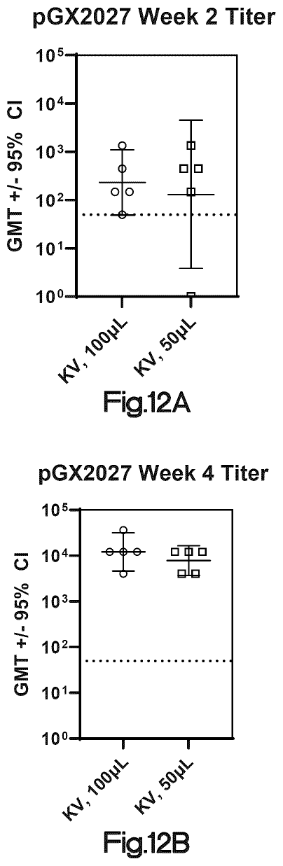

Referring now to FIGS. 17A-17B, a follow-up study replicates the study disclosed above with reference to FIG. 16A-16B evaluating immune responses in rabbits, but using a different plasmid (this time using plasmid 2303 at a DNA concentration of 3.0 mg/mL). All other parameters of this study were the same as in the study shown in FIGS. 16A-16B. In particular, FIG. 17A shows titer data at Week 0 (and is comparable to FIG. 16A), and FIG. 17B shows titer data at Week 2 (and is comparable to FIG. 16A). As with the previous study in rabbits, the present follow-up study again demonstrates that, for immune response, KV treatments (Group 3) outperformed VEP treatments (Group 4), with the latter being generally equivalent to NEP treatments (Group 5). In the present follow-up study, the INJ treatments (Group 1) was only slightly less immunogenic than SV treatments (Group 2), which was itself less immunogenic than the VEP and NEP treatments (Groups 4, 5), whereas in the previous study (FIGS. 16A-16B), the SV treatments had similar immunogenicity to the VEP and NEP treatments.

### Study 11

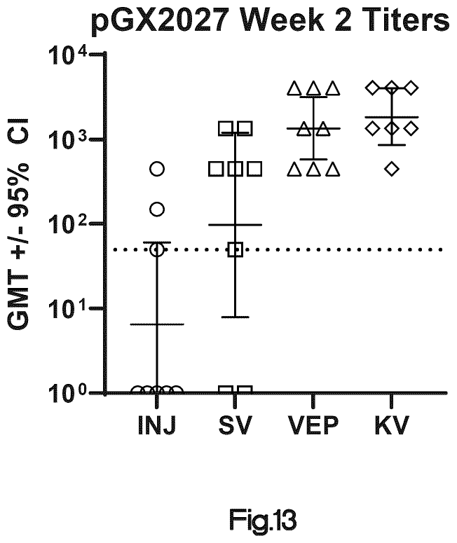

Referring now to FIG. 18, this study evaluates the impact that hyaluronidase has on kinetic vacuum (KV) and static vacuum (SV) treatments in guinea pigs, further in comparison with vacuum electroporation (VEP) treatments.

Subjects were injected intradermally with uniform volumes of a plasmid and were then treated according to the following treatment groups:
Group 1: SV treatment using a centerpost vacuum cup 102 design (see FIGS. 2A-2C);
Group 2: VEP treatment using a centerpost-electrode VEP cup 402 (see FIGS. 4A-4B);
Group 3: KV treatment (Pattern A, 3 cycles) using the same centerpost vacuum cup design used in Group 1;
Group 4: KV treatment using the cup design and kinetic movements as in Group 3, but with hyaluronidase added to the injection; and
Group 5: SV treatment with hyaluronidase added to the injection, but otherwise using the same SV treatment and cup of Group 1.

Groups 1-3 involved six (6) samples each, while Groups 4-5 involved seven (7) samples each. Each sample was administered one (1) ID injection over the left flank. Each injection contained plasmid 9517 at a volume of 100 uL with a DNA concentration of 0.25 mg/mL. Further details regarding the test parameters for this study are shown in Table 9 below:

TABLE 9

| Group Number | | | |
|---|---|---|---|
| (n/group) | Plasmid | Treatment | HYA |
| 1. (n = 6) | 9517 | SV | − |
| 2. (n = 6) | 9517 | VEP | − |
| 3. (n = 6) | 9517 | KV | − |
| 4. (n = 7) | 9517 | KV | + |
| 5. (n = 7) | 9517 | SV | + |

Titer data for all Groups is shown in FIG. 18. In this study, the KV treatments (both with and without hyaluronidase) produced greater ELISA responses than the VEP treatments. Additionally, in this study, hyaluronidase enhanced binding titers for KV treatments but not for SV treatments. In fact, the SV treatments produced virtually no detectable immune responses in this study, with or without hyaluronidase.

### Study 12

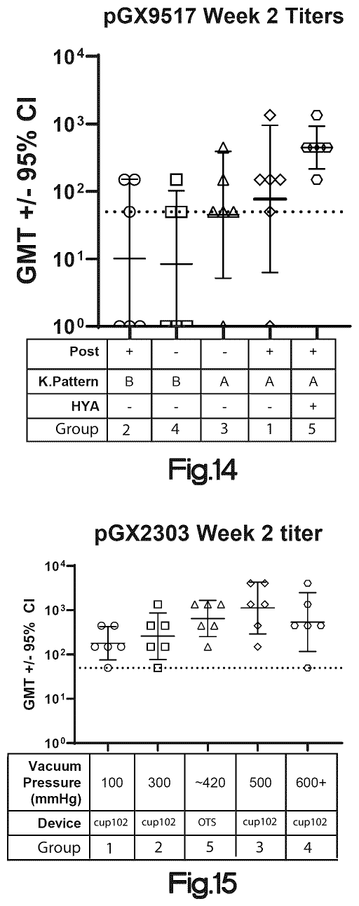

Referring now to FIG. 19, this study evaluates the impacts that different numbers of kinetic vacuum (KV) movements and, separately, skin thickness have on immune response (ELISA expression) in guinea pigs.

Subjects were injected intradermally with uniform volumes of a plasmid and were then treated according to the following treatment groups, each administering KV treatments using a centerpost vacuum cup 102 design (see FIGS. 2A-2C) moving the cup according to Pattern A (linearly back-and-forth across the injection site), but varying the number of cycles and the injection site:
Group 1: injection in the right flank, followed by Pattern A, 2 cycles (four (4) total swipes) with the vacuum cup 102;
Group 2: injection in the right flank, followed by Pattern A, 3 cycles (six (6) total swipes) with the vacuum cup 102;
Group 3: injection in the right flank, followed by Pattern A, 4 cycles (eight (8) total swipes) with the vacuum cup 102;
Group 4: injection in the back (where the subjects' skin is thicker than at the flank), followed by Pattern A, 3 cycles (six (6) total swipes) with the vacuum cup 102; and
Group 5: injection in the belly (where the subjects' skin is thinner than at the flank, followed by Pattern A, 3 cycles (six (6) total swipes) with the vacuum cup 102.

Groups 1-3 involved six (6) samples each, while Groups 4-5 involved seven (7) samples each. Each injection contained plasmid 2303, at a volume of 100 uL, and a DNA concentration of 0.3 mg/mL for each Group. Further details regarding the test parameters for this study are shown in Table 10 below:

TABLE 10

| Group Number | | | | # | Skin Tx |
|---|---|---|---|---|---|
| (n/group) | Plasmid | Treatment | Movement Cycle Pattern | Cycles | Location |
| 1. (n = 6) | 2303 | KV | (0, 0)→(t, 0)→(0, 0)→(−t, 0)→(0, 0) | 2 | Flank |
| 2. (n = 6) | 2303 | KV | (0, 0)→(t, 0)→(0, 0)→(−t, 0)→(0, 0) | 3 | Flank |
| 3. (n = 6) | 2303 | KV | (0, 0)→(t, 0)→(0, 0)→(−t, 0)→(0, 0) | 4 | Flank |
| 4. (n = 7) | 2303 | KV | (0, 0)→(t, 0)→(0, 0)→(−t, 0)→(0, 0) | 3 | Back |
| 5. (n = 7) | 2303 | KV | (0, 0)→(t, 0)→(0, 0)→(−t, 0)→(0, 0) | 3 | Belly |

Combined titer data for all Groups is shown in FIG. 19. In this study, differing the number of linear translations or swipes between 4-8 swipes across the injection site did not meaningfully impact binding titers. Moreover, the location of the treatment site (thin skin versus thick skin) did not appear to produce a meaningful difference in immunogenicity.

### Study 13

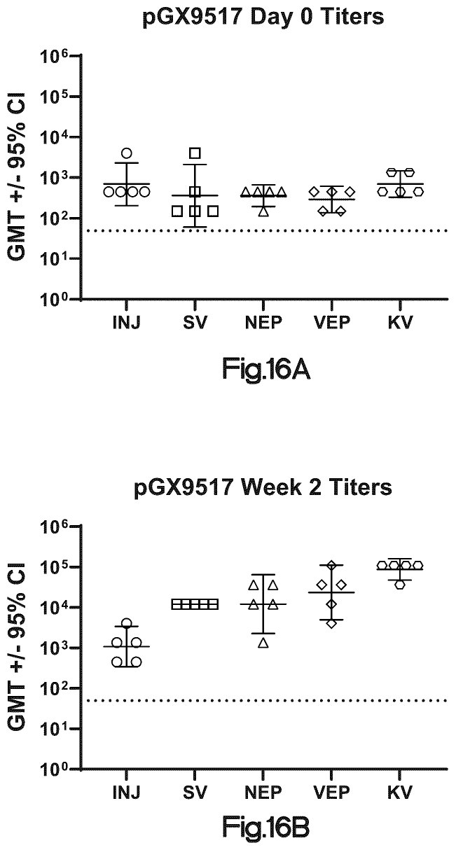

Referring now to FIGS. 20A-20B, this study evaluates the impacts that injection volume and hyaluronidase have on gene (GFP) expression following static vacuum (SV) and kinetic vacuum (KV) treatments administered to guinea pigs.

Subjects in six (6) Groups were injected intradermally on the right flank with a plasmid encoding the gene for GFP and were then treated according to the following treatment groups, each group being administering KV or SV treatments using a centerpost vacuum cup 102 design (see FIGS. 2A-2C) having a chamber diameter D1 of 12 mm:
Group 1: SV treatment after injection volume of 100 uL;
Group 2: SV treatment, same as Group 1 but with hyaluronidase;
Group 3: KV treatment after injection volume of 100 uL, the KV treatment used Pattern A, 3 cycles, with the vacuum cup 102;
Group 4: KV treatment, same as Group 3 but with hyaluronidase;
Group 5: KV treatment, same as Group 4 but using a plasmid injection volume of 400 uL;
Group 6: SV treatment, same as Group 2 but using a plasmid injection volume of 400 uL;

Groups 1 and 3 involved eight (8) samples each, Groups 2 and 4 involved twelve (12) samples each, and Groups 5 and 6 involved four (4) samples each. The following groups were paired together on test subjects: Groups 1-2, Groups 3-4, such that each group pair performed treatments on the same subjects side by side. Groups 5 and 6 were not paired with any other group on a test subject. Each injection contained plasmid 5013 (which encodes the gene for GFP) having a DNA concentration of 0.5 mg/mL. Further details regarding the test parameters for this study are shown in Table 11 below:

TABLE 11

| Group Number | | | Injection | |
|---|---|---|---|---|
| (n/group) | Plasmid | Treatment | Vol. (μL) | HYA |
| 1. (n = 8) | 5013 | SV | 100 | − |
| 2. (n = 12) | 5013 | SV | 100 | + |
| 3. (n = 8) | 5013 | KV | 100 | − |
| 4. (n = 12) | 5013 | KV | 100 | + |
| 5. (n = 4) | 5013 | KV | 400 | + |
| 6. (n = 4) | 5013 | SV | 400 | + |

GFP for the groups is shown in FIG. 20A, with all images at the same scale and same image settings (e.g., exposure time). GFP quantification for the groups is shown in FIG. 20B. In this study, KV treatments (Groups 3-5) significantly increased gene expression over SV treatments (Groups 1-2 and 6). Additionally, within the 100-uL injection volume groups (Groups 1-4), the KV treatments benefitted dramatically by the addition of hyaluronidase (compare Group 3 with Group 4), whereas the SV treatments that included hyaluronidase produced less gene expression on average than those without hyaluronidase (compare Group 2 with Group 1). Within the 400 uL injection volume (with hyaluronidase) groups (Groups 5 and 6), KV treatments (Group 5) produced significantly greater gene expression, and over a significantly larger expression area, compared to the SV treatments (Group 6). Within the KV kinetic vacuum treatments using hyaluronidase, the 400 uL injection volume treatments (Group 5) had a dramatic increase in gene expression area compared to the 100 uL treatments (Group 4). In the static vacuum treatments with hyaluronidase, the 400 uL treatments (Group 6) showed a mild improvement in gene expression over the 100 uL treatments (Group 2).

### Study 14

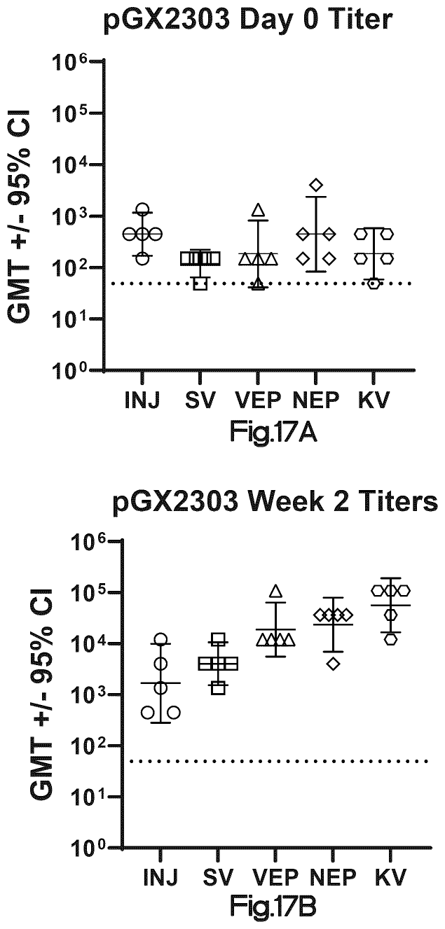

Referring now to FIG. 21, a further study evaluates the impact that high injection volumes with and without hyaluronidase have on immune response (ELISA expression) in guinea pigs that were administered kinetic vacuum (KV) or static vacuum (SV) treatments.

Subjects in four (4) Groups were injected intradermally on the left flank with a plasmid and were then treated according to the following vacuum treatment groups that each used a centerpost vacuum cup 102 design (see FIGS. 2A-2C) having a chamber diameter D1 of 12 mm:
Group 1: KV treatment after injection volume of 100 uL without hyaluronidase;
Group 2: KV treatment, same as Group 1 (no hyaluronidase) but with an injection volume of 400 uL;
Group 3: KV treatment after injection volume of 400 uL with hyaluronidase; and
Group 4: SV treatment, otherwise the same as Group 3 (400 uL with hyaluronidase).

All groups in this study involved five (5) samples each, with each injection containing plasmid 2303 having a DNA concentration of 0.3 mg/mL. Further details regarding the test parameters for this study are shown in Table 12 below:

TABLE 12

| Group Number | | | Injection | |
|---|---|---|---|---|
| (n/group) | Plasmid | Treatment | Vol. (μL) | HYA |
| 1. (n = 5) | 2303 | KV | 100 | − |
| 2. (n = 5) | 2303 | KV | 400 | − |
| 3. (n = 5) | 2303 | KV | 400 | + |
| 4. (n = 5) | 2303 | SV | 400 | + |

Combined titer data for all Groups is shown in FIG. 21. In this study, the treatments without hyaluronidase (Groups 1-2: KV after 100 μL and 400 uL injection volumes, respectively) had similar immune responses. The KV treatments after 400 uL injection volumes with hyaluronidase (Group 3) had the strongest immune response of all the groups in this study. The SV treatments after 400 uL injection volumes with hyaluronidase (Group 4) had the weakest immune response of all groups in this study, which was unexpected, particularly in light of the results from the study discussed above with reference to FIG. 20. The results in the present study shown in FIG. 21 suggest that the KV treatments may have an additive, adjuvanting effect on top of the raw gene expression benefit.

### Study 15

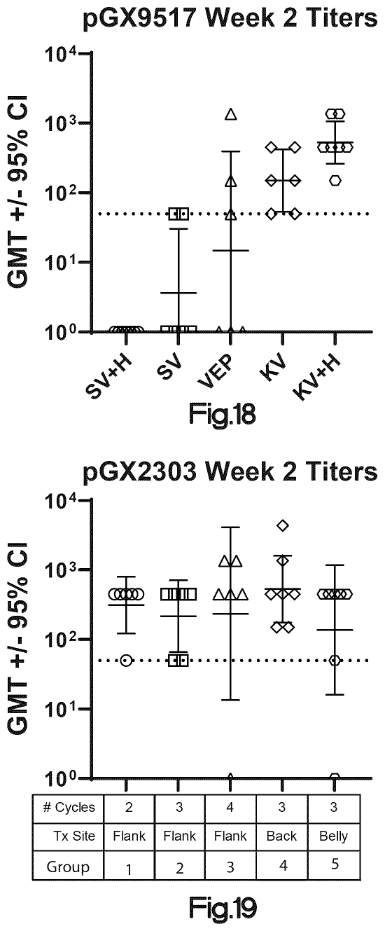

Referring now to FIG. 22, a study was conducted to evaluate the effects that combining kinetic vacuum (KV) treatments with vacuum electroporation (VEP) have on immune response (ELISA expression) in guinea pigs.

Subjects in four (4) Groups were injected intradermally on the left flank with uniform volumes of a plasmid and were then treated according to the following vacuum treatment groups, each group using a centerpost-electrode VEP cup 402 (see FIGS. 4A-4B) having a chamber diameter D1 of 12 mm:
Group 1: KV treatment only, administering three (3) sets of swipes (six (6) total swipes) with the vacuum cup;
Group 2: VEP treatment followed by the same KV treatment as Group 1;
Group 3: the same KV treatment as Group 1, followed by the same VEP treatment as in Group 2 (i.e., Group 3 used the same KV and VEP treatments in Group 2 but in the reserve sequence); and

All groups in this study involved five (5) samples each, with each injection containing plasmid 9517 having a DNA concentration of 0.25 mg/mL. Vacuum pressure for each treatment was −500 mmHg. Further details regarding the test parameters for this study are shown in Table 13 below:

TABLE 13

| Group Number | | Treatment | Treatment |
|---|---|---|---|
| (n/group) | Plasmid | Step 1 | Step 2 |
| 1. (n = 5) | 9517 | KV | N/A |
| 2. (n = 5) | 9517 | VEP | KV |
| 3. (n = 5) | 9517 | KV | VEP |

Combined titer data for all Groups is shown in FIG. 22. In this study, adding VEP before or after the KV treatment did not provide any additional immune benefit, and trended toward decreased immune response.

### Study 16

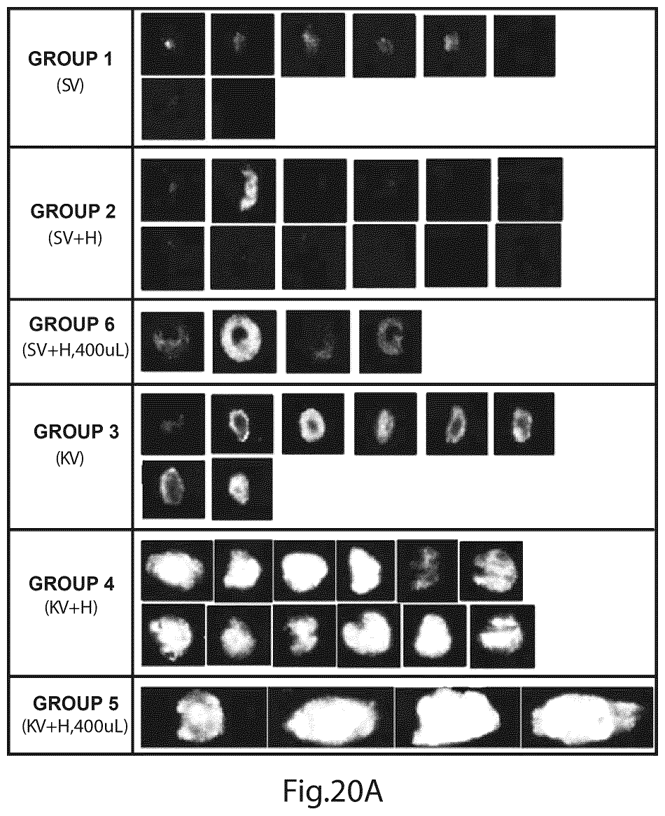

Referring now to FIGS. 23A-23B, a study was conducted in guinea pigs to compare the immune responses (ELISA expression), cellular responses (ELISpot data), and neutralization produced by kinetic vacuum (KV) treatments versus mRNA/lipid nanoparticle injection-only (INJ) treatments and needle electroporation (NEP) treatments.

Subjects received plasmid injections and treated according to the following treatment groups:
Group 1: NEP treatment after injection of plasmid 9501, wherein the NEP used the CELLECTRA™ 2000-3P device;
Group 2: KV treatment after injection of plasmid 9501, wherein the KV treatment used a centerpost vacuum cup 102 (see FIGS. 2A-2C) having a chamber diameter D1 of 12 mm;
Group 3: KV treatment, the same as Group 2 but with hyaluronidase added to the injection;
Group 4: INJ treatment of mRNA-1273 (an mRNA-based vaccine for SARS-CoV-2), produced by Moderna Inc., based in Cambridge, Massachusetts, United States.

Each Group involved five (5) samples, each sample receiving one (1) injection. Groups 1-3 received intradermal (ID) injections of the plasmid (9501) at an injection volume of 100 uL, with a DNA dose of 100 ug, and a DNA concentration of 1.0 mg/mL, in the skin over the left flank; Group 4 received intramuscular (IM) injections of the plasmid (mRNA-1273) at an injection volume of 50 uL, with a DNA dose of 10 ug, and a DNA concentration of 0.2 mg/mL, in the left quadriceps. Further details regarding the test parameters for this study are shown in Table 14 below:

TABLE 14

| Group | | | | | Inj. | DNA |
|---|---|---|---|---|---|---|
| Number | | Drug | | | Vol. | Dose |
| (n/group) | Agent | Type | Treatment | HYA | (μL) | (μg) |
| 1. (n = 5) | 9501 | DNA | NEP | − | 100 | 100 |
| 2. (n = 5) | 9501 | DNA | KV | − | 100 | 100 |
| 3. (n = 5) | 9501 | DNA | KV | + | 100 | 100 |
| 4. (n = 5) | mRNA-1273 | mRNA | Lipid | − | 50 | 10 |
| | | | Nanoparticles | | | |

FIGS. 23A and 23B show spike binding data for all groups at Week 2 (after the first dose/treatment) and at Week 5 (after the second dose/treatment), respectively. FIG. 23C shows ELISpot data for all groups at Week 5 (after the second dose/treatment). FIG. 23D shows SARS-COV-2 pseudovirus neutralization data for all groups at Day 0 and Day 14 (after the first dose/treatment).

In this study, in terms of binding titer results, as shown in FIGS. 23A-23B, the mRNA vaccine (Group 4) outperformed KV plus hyaluronidase treatments (Group 3), which outperformed KV treatments without hyaluronidase (Group 2), which outperformed NEP treatments (Group 1). In terms of cellular responses, as shown in FIG. 23C, each of the DNA plasmid treatments (Groups 1-3) outperformed the mRNA vaccine (Group 4). In terms of neutralization, as shown in FIG. 23D, the mRNA vaccine (Group 4) outperformed the KV plus hyaluronidase treatments (Group 3), which outperformed the KV treatments without hyaluronidase (Group 2), which was generally equivalent to the NEP treatments (Group 1).

In this study, KV plus hyaluronidase treatments (Group 3) achieved the closest to mRNA-level (Group 4) humoral binding responses (FIGS. 23A-23B) and pseudovirus neutralization responses (FIG. 23D), followed by KV without hyaluronidase (Group 2), with NEP treatments (Group 1) generating the weakest humoral responses (FIG. 23C). For neutralization (FIG. 23D), only KV plus hyaluronidase treatments (Group 3) and the mRNA vaccine (Group 4) generated 100% neutralizing activity after one vaccination. Regarding cellular responses (FIG. 23C), the DNA treatments (Groups 1-3) offered generally stronger cellular responses than the mRNA vaccine (Group 4), though KV plus hyaluronidase treatments (Group 3) trended towards slightly higher responses than NEP treatments (Group 1) for the wildtype assay.

### Study 17

Referring now to FIG. 24, a study evaluated whether a single kinetic vacuum (KV) treatment can effectively cause transfection of multiple injections (blebs). Subjects in this study were evaluated after receiving intradermal injections of various partitions of a uniform total combined volume of two (2) separate plasmids (one encoding the gene for green fluorescent protein (GFP) and the other encoding the gene for red fluorescent protein (RFP)) and then administer a single KV treatment over the blebs. The subjects were evaluated according to four (4) treatment groups:
Group 1 (Control): KV treatment of one (1) injection of plasmid 5013 (which encodes the gene for GFP) at an injection volume of 100 uL;
Group 2: KV treatment of two (2) side-by-side injections, one of plasmid 5013 and the other of plasmid 9902 (which encodes the gene for RFP), each at an injection volume of 50 uL;
Group 3: KV treatment of four (4) injections in a square pattern, in which the top row of injections used plasmid 5013 and the bottom row of injections used plasmid 9902, each injection at a volume of 25 uL; and
Group 4: KV treatment of four (4) injections in a linear row of alternating plasmids 5013 and 9902, each injection at a volume of 25 uL.

Each Group involved five (5) replicates, each replicate receiving their respective injection(s) in skin over the flank. Each injection included hyaluronidase (HYA). The KV treatment for each group employed Pattern A, 3 cycles, except for Group 3, which employed a modified version of Pattern A. In particular, for Group 3, the first position of the vacuum chamber was intermediate the left blebs in the top and bottom rows. When vacuum pressure was applied for Group 3, the chamber diameter was large enough so that the left blebs in the top and bottom rows were both drawn into the vacuum chamber prior to performing the kinetic vacuum translations, which translations were performed side-to-side in a manner that was otherwise consistent with Pattern A (3 cycles). For the multi-injection groups (Groups 2-4), the use of plasmids encoding GFP and RFP were employed to help visualize differences in expression for each injection site, and to help visualize the extent to which different injection sites may or may not blend together. Further details regarding the test parameters for this study are shown in Table 15 below:

TABLE 15

| Group | | | | | Bleb | |
|---|---|---|---|---|---|---|
| Number | | Treat- | | # of | Volume | |
| (n/group) | Plasmid(s) | ment | HYA | Blebs | (μL) | Bleb Pattern |
| 1. (n = 5) | 5013 | KV | + | 1 | 100 | Single Bleb |
| 2. (n = 5) | 5013, | KV | + | 2 | 50 | Two blebs in a |
| | 9902 | | | | | row-horizontal |
| 3. (n = 5) | 5013, | KV | + | 4 | 25 | Four blebs in a |
| | 9902 | | | | | square pattern |
| 4. (n = 5) | 5013, | KV | + | 4 | 25 | Four blebs in a |
| | 9902 | | | | | row-horizontal |

From the results shown in FIG. 24, it can be seen that a single KV treatment was able to transfect multiple injection sites in each group, including in subjects in the four-injection groups (Groups 3 and 4). This study demonstrates that the KV treatments herein can be employed successfully with injection partitioning in terms of gene expression.

### Study 18

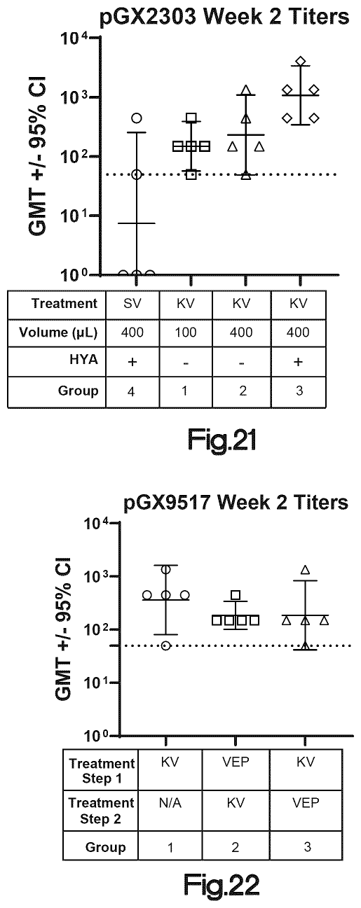

Referring now to FIG. 25, a study evaluated whether the same kinetic vacuum (KV) treatments performed by different individuals (operators) would produce different immune responses (ELISA expression) in guinea pigs. Subjects were injected intradermally in flank skin with uniform volumes of plasmid 2027 and were then treated according to Groups 1-4, wherein the subjects in each group were treated by a separate individual (i.e., Group 1 subjects were treated by Operator 1, Group 2 subjects were treated by Operator 2, etc.). Each Group involved five (5) replicates, with each Operator performing the KV treatment using Pattern A, 3 cycles, with a centerpost vacuum cup 102 (see FIGS. 2A-2C) having a chamber diameter D1 of 12 mm. The results demonstrate that there was no significant Operator-driven difference in ELISA expression at 2 weeks when the same KV treatment pattern was employed.

### Study 19

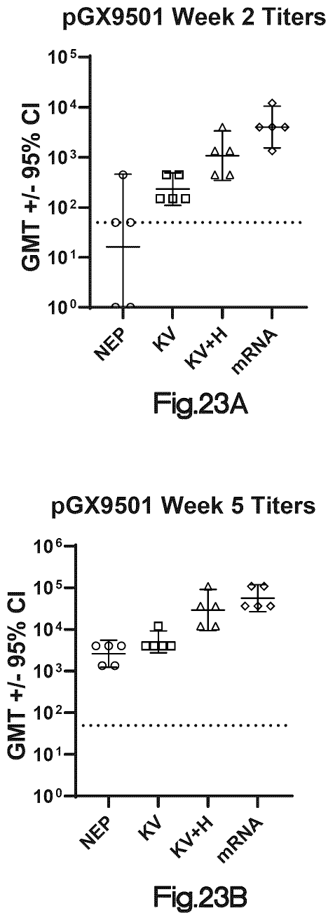

Referring now to FIGS. 26A-26C, a study was conducted in guinea pigs to compare the immune responses (ELISA expression) and neutralization produced by DNA-launched nanoparticles (DLNP) with kinetic vacuum (KV) treatments versus mRNA injection-only (INJ) treatments.

Subjects received plasmid injections and were treated according to the following treatment groups:
Group 1: KV treatment after injection of DLNP-based plasmid 9528 formulated with hyaluronidase (HYA), wherein the KV treatment employed Pattern A, 3 cycles using a centerpost vacuum cup 102 (see FIGS. 2A-2C) having a chamber diameter D1 of 12 mm; and
Group 2: INJ treatment of mRNA-1273.

Each Group involved five (5) subjects, each subject receiving one (1) injection. Group 1 received intradermal (ID) injections of the plasmid (9528) at an injection volume of 100 uL, with a DNA dose of 10 ug, and a DNA concentration of 0.1 mg/mL, formulated with HYA at a dose of 135 U/mL, in the skin over the flank; Group 2 received intramuscular (IM) injections of the plasmid (mRNA-1273) at an injection volume of 50 uL, with an mRNA dose of 10 ug, and an mRNA concentration of 0.2 mg/mL, in the tibialis anterior muscle.

Further details regarding the test parameters for this study are shown in Table 16 below:

TABLE 16

| Group Number | | | |
|---|---|---|---|
| (n/group) | Drug | Treatment | HYA |
| 1. (n = 5) | 9528 | KV | + |
| 2. (n = 5) | mRNA-1273 | Lipid Nanoparticles | − |

FIG. 26A shows spike binding data for both groups at Day 14 (Week 2), Day 28 (Week 4) and Day 46 (about Week 6 ½) post-treatment, with the first dose/treatment administered at Day 0 and a second dose/treatment administered at Day 21. FIGS. 26B-26C show SARS-COV-2 pseudovirus neutralization data for both groups over a period exceeding 300 days.

In this study, in terms of binding titer results, as shown in FIG. 26A, the DLNP plus KV plus HYA treatments performed comparably with the mRNA vaccine over the long course of this study. Furthermore, in terms of neutralization, as shown in FIGS. 26B-26C, the DLNP plus KV plus HYA treatments showed more stable neutralizing titers than the mRNA vaccine and maintained a higher level of neutralization for nearly one (1) year. Notably, the magnitude of the neutralizing titers for mRNA-1273 fell to less than 5% of their peak throughout the monitoring period, whereas DLNP plus KV plus HYA injections maintained 34% of peak response through the last time point measured. Though mRNA-1273 elicited higher peak neutralizing antibodies, the difference in this response drop-off between mRNA and DLNP with KV led to equivalent neutralization at approximately 100 days post-first vaccination, after which DLNP with KV exhibited superior titers than mRNA.

### Study 20

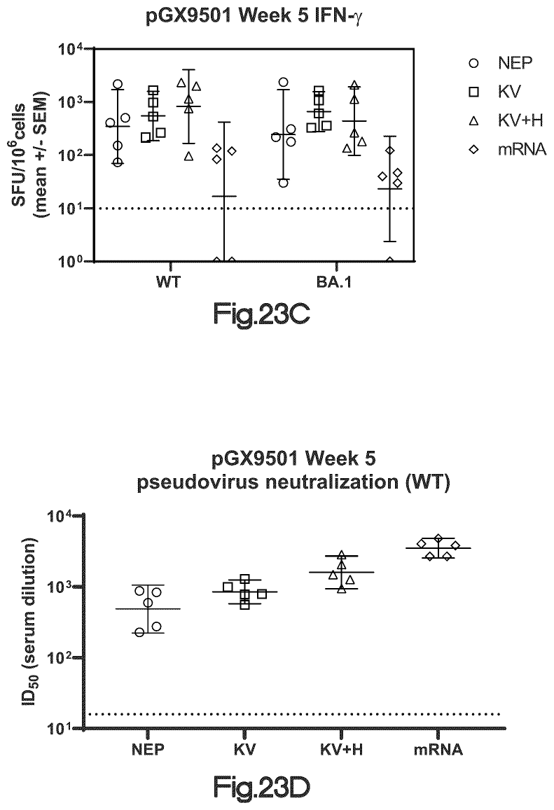

Referring now to FIG. 27, a study was conducted to compare the spreading effect that kinetic vacuum (KV) treatments have on an injected bolus in skin tissue (a bleb) compared to that produced by static vacuum (SV) treatments.

Subjects in each group were injected with a dye (Trypan Blue) in flank skin and then treated according to the following treatment groups: Group 1 received SV treatment; Group 2 received KV treatment employing Pattern A, 3 cycles, using a centerpost vacuum cup 102 (see FIGS. 2A-2C) having a chamber diameter D1 of 12 mm; and Group 3's injections included hyaluronidase (HYA) but otherwise received the same treatment as Group 2. Each Group involved five (5) subjects. Bleb diameters were measured in each subject after the injection, once before treatment and again after treatment to compare the physical spreading effect produced by KV and SV treatments. The spreading effect is quantified as a ratio of the pre-treatment vs. post-treatment diameter. The blue dye assisted with identifying the extent of spread during the measurement phase.

As shown, the SV treatments (Group 1) produced only a marginal spreading effect; the KV treatments (Group 2) produced a positive spreading effect; and the KV plus HYA treatments (Group 3) significantly outperformed Groups 1 and 2.

### Study 21

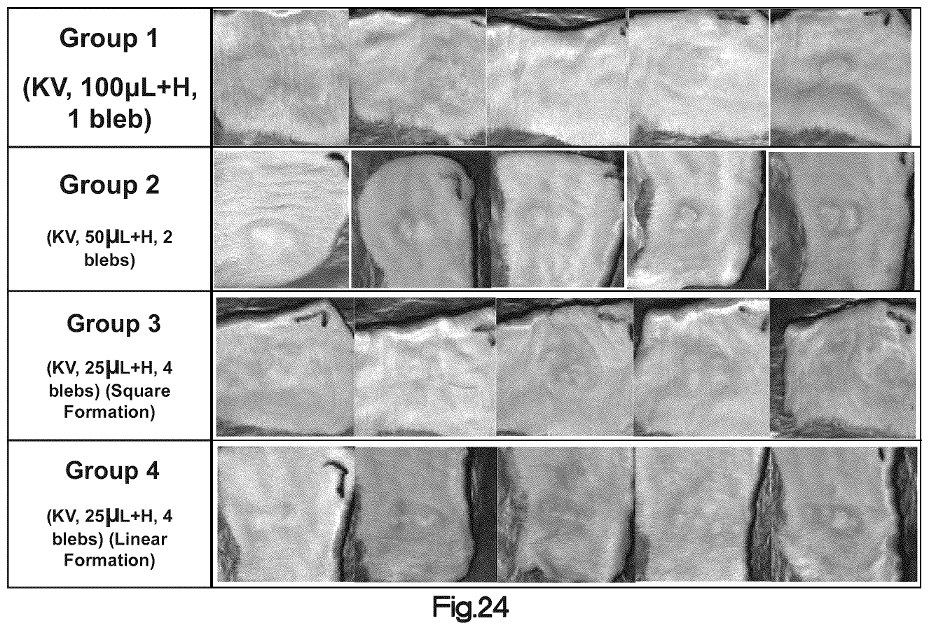

Referring now to FIGS. 28A, a study was conducted to compare immune responses (ELISA expression) in guinea pigs following kinetic vacuum (KV) treatments performed by different devices, particularly one (1) centerpost vacuum cup 102 (see FIGS. 2A-2C) and four (4) off-the-shelf (OTS) vacuum devices, all having similar chamber sizes and applying similar vacuum pressures.

All subjects in this study received injections of the same plasmid (2303) at the same dosage in skin over the flank. Subjects were grouped according to vacuum cup: (1) “cup 102”—a centerpost vacuum cup 102 (see FIGS. 2A-2C) having a chamber diameter of 12 mm; (2) OTS-1 device; (3) OTS-2 device; (4) OTS-3 device; and (5) OTS-4 device. Further details regarding the test parameters for this study are shown in Table 17 below:

TABLE 17

| | | Chamber | Measured Vacuum |
|---|---|---|---|
| Device | Cup Shape | Diameter (mm) | Pressure (mmHg) |
| cup102 | Circle | 12 | −500 |
| OTS-1 | Circle | 10 | −508 |
| OTS-2 | Circle | 9.5 | −474 |
| OTS-3 | Rounded | 7.75 × 6.33 | −474 |
| | Rectangle | | |
| OTS-4 | Circle | 8.8 | −490 |

Each Group involved five (5) subjects, and each KV treatments employed Pattern A, 3 cycles. Titer data is shown for the groups at Week 2. The results show that the five (5) vacuum devices produced similar immunogenic responses when using similar chamber sizes and applying similar vacuum pressures.

### Study 22

Referring now to FIG. 28B, a study similar to Study 21 (FIG. 28A) was conducted to determine the effect of cup size on immune responses (ELISA titers) in guinea pigs following kinetic vacuum (KV) treatments. All tests in this study were performed using the OTS-4 device (see Table 0), with five (5) different vacuum cups coupled interchangeably thereto. Using the same OTS-4 device with different interchangeable cups helped maintain uniform vacuum pressure while using different chamber sizes and geometries, which are found in Table 18 below:

TABLE 18

| Group Number | | | | Chamber Diameter |
|---|---|---|---|---|
| (n/group) | Plasmid | Delivery | Cup Shape | (mm) |
| 1. (n = 5) | 9501 | KV | Circle | 11.75 |
| 2. (n = 5) | 9501 | KV | Circle | 5.8 |
| 3. (n = 5) | 9501 | KV | Circle | 3.8 |
| 4. (n = 5) | 9501 | KV | Ellipse | 8.3 × 3.0 |
| 5. (n = 5) | 9501 | KV | Circle | 8.8 |

All subjects in this study received injections of the same plasmid (9501) at the same dosage in skin over the flank. Subjects were grouped according to the vacuum cup used. Each Group involved five (5) subjects, and each KV treatment employed Pattern A, 3 cycles. Titer data is shown for the groups at Week 2. For the cup sizes tested, the results show a strong correlation whereby immune response increased with cup size. At the lower end of cup size, the smallest cup tested (Group 3:3.8 mm chamber diameter) failed to produce a meaningful immune response. These results demonstrate that, in KV treatments using similar vacuum pressures and movement patterns, cup size is a significant factor in immune response.

### Study 23

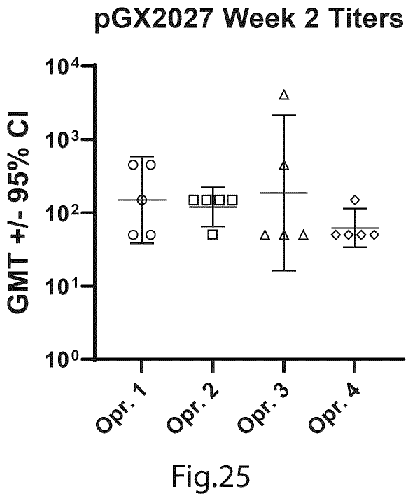

Referring now to FIG. 29, a study was conducted to determine whether partitioning a given injection volume into smaller volumes produced an immunogenic effect in guinea pigs following kinetic vacuum (KV) treatment over those injection sites.

Subjects received injections of plasmid 2027 and were treated according to the following treatment group:
Group 1: total injection volume of 100 μL in one (1) injection (i.e., 1 bleb partition);
Group 2: total injection volume of 100 μL partitioned over three (3) injections (i.e., 3 bleb partitions of 33.3 μL each);
Group 3: total injection volume of 300 μL in one (1) injection (i.e., 1 bleb partition); and
Group 4: total injection volume of 300 μL partitioned over nine (9) injections (i.e., 9 bleb partitions of 33.3 L each).

Each Group involved five (5) subjects, each subject being injected in flank skin then administer KV treatment using the OTS-3 device (see Table 0), employing Pattern A, 3 cycles. For those Groups having multiple bleb partitions (i.e., Groups 2 and 4), the KV treatment involved a single treatment performed over all bleb partitions in the group (i.e., in each cycle of Pattern A, the vacuum cup was moved over all bleb partitions in the group). Further details regarding the test parameters for this study are shown in Table 19 below:

TABLE 19

| | | | Total | | Volume |
|---|---|---|---|---|---|
| Group Number | | | Volume | Bleb | per Bleb |
| (n/group) | Plasmid | Treatment | (μL) | Partitions | (μL) |
| 1. (n = 5) | 2027 | KV | 100 | 1 | 100 |
| 2. (n = 5) | 2027 | KV | 100 | 3 | 33.3 |
| 3. (n = 5) | 2027 | KV | 300 | 1 | 300 |
| 4. (n = 5) | 2027 | KV | 300 | 3 | 33.3 |

Titer data is shown for the groups at Week 2. For Groups 1-2, the results do not show any immunologic effect by partitioning the 100 μL injection volume into three (3) injections, although this may be due to immune responses that were below detectable levels and not necessarily due to a lack immunologic difference between Groups 1 and 2. For the higher total volume groups (Groups 3-4), partitioning the 300 μL injection volume into nine (9) injections can be seen to have caused a higher trend in immune response. These results show a trend that injection volume partitioning with KV treatments in high volume blebs may increase immune responses. These results also support other test results and data showing that KV treatments are effective in enhancing immune response so long as the treatment involves passing the vacuum cup over the entire injection zone.

### Study 24

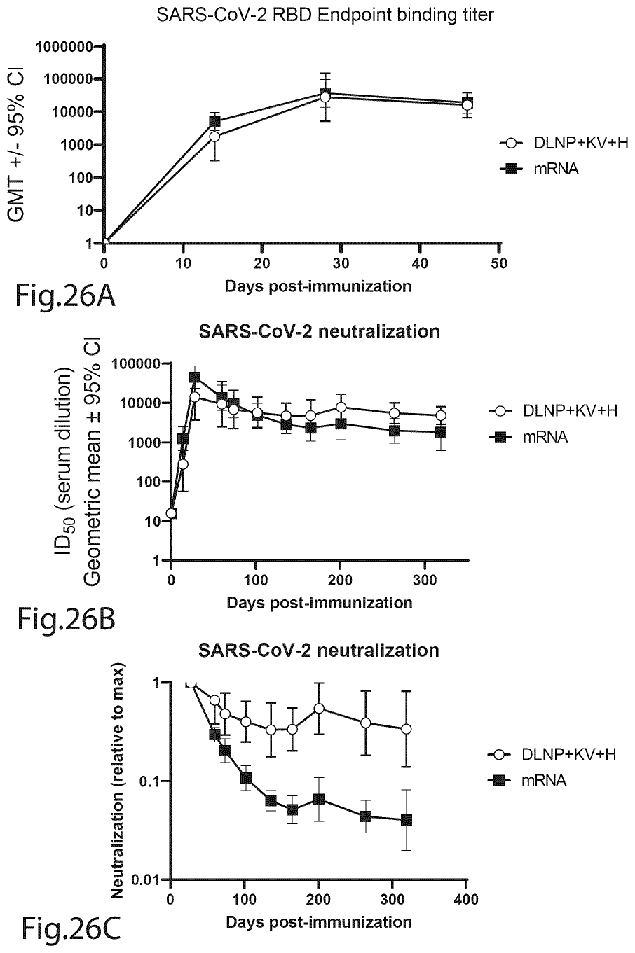

Referring now to FIG. 30, another study explores the effect of reducing DNA dosage (via reduction in injection volume) with kinetic vacuum (KV) treatment on immune responses (binding ELISA responses) in guinea pigs. Compare with Study 5 (FIGS. 12A-12B). In the present study, however, needle electroporation (NEP) treatments were also tested for comparison with KV treatments in reduced-dosage scenarios.

In this study, subjects in two groups (Group 1 and Group 2, each group with five (5) subjects) were injected intradermally in the flank with the same plasmid (9501) at uniform volumes (100 uL), uniform DNA doses (2.5 ug), and formulated with hyaluronidase (HYA) then administered either KV treatment (Group 1) or NEP treatment (Group 2). The KV treatment was administered using a centerpost vacuum cup 102 (see FIGS. 2A-2C) having a chamber diameter D1 of 12 mm, employing Pattern A, 3 cycles.

The ELISA responses at Week 2 are shown for both Groups in FIG. 30 (with the dashed line indicating the limit of detectable titers). The results show that, at the low doses tested, the KV treatment group produced a measurable immune response, while the NEP treatment Group did not. These results also provide evidence that KV treatments can have dose-sparing benefits compared to NEP.

### Study 25

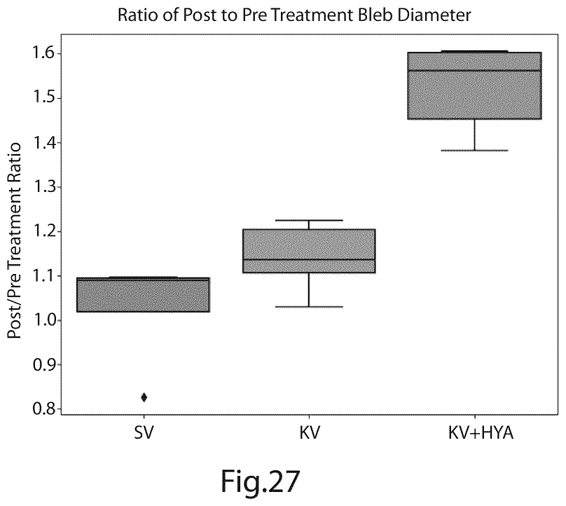

Referring now to FIG. 31, yet another study was conducted to explore the effect of reducing DNA dosage (via reduction in injection volume) with kinetic vacuum (KV) treatment on immune responses (binding ELISA responses) in guinea pigs. Compare with Study 24 (FIG. 30). In the present study, subjects in four (4) groups (five (5) subjects per group) received intradermal injections of plasmid 2303 in flank skin, wherein each successive group received one-half the dosage of the previous group. Group 1 received an injection volume of 100 uL with a DNA-dose of 30 ug; Group 2 received an injection volume of 50 uL with a DNA-dose of 15 ug; Group 3 received an injection volume of 25 uL with a DNA-dose of 7.5 ug; and Group 4 received an injection volume of 12.5 uL with a DNA-dose of 3.75 ug. All other factors were constant for each group: KV treatments were administered by the OTS-3 device (see Table 17), employing Pattern A, 3 cycles.

The ELISA responses at Week 2 are shown for all Groups. Interestingly, no measurable drop in ELISA immunogenicity was observed in the reduced-dosage groups, which provides further evidence that KV treatments have potential dose-sparing benefits.

### Study 26

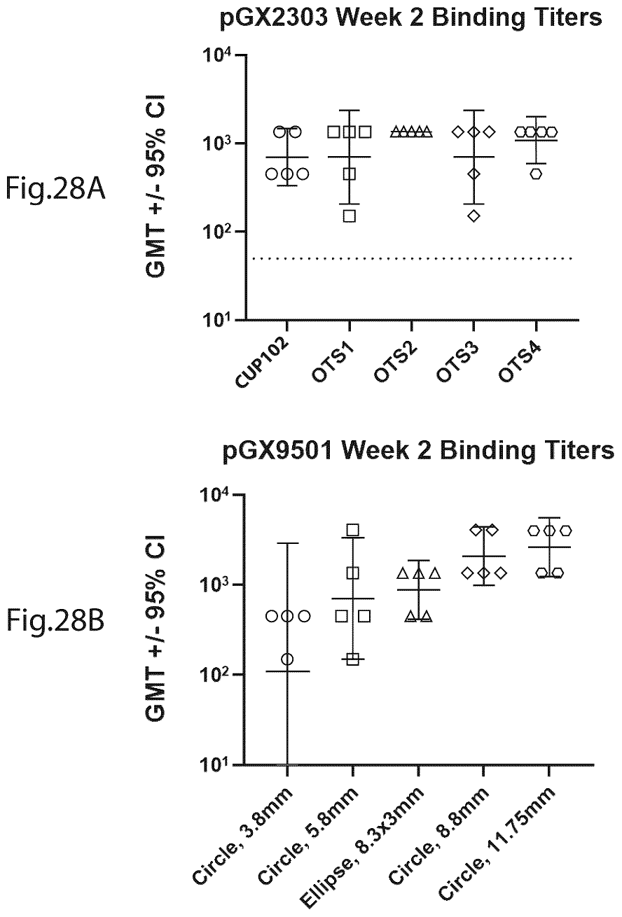

Referring now to FIG. 32A-32E, a study was conducted to compare immune responses, particularly ELISA responses (FIGS. 32A-32C) and T-cell responses (FIGS. 32D and 32E), in rabbits following treatments involving kinetic vacuum (KV), needle electroporation (NEP), and intramuscular electroporation (IM-EP).

In this study, subjects received injections of plasmid 9501 (which encodes for the entire length of the Spike glycoprotein of SARS-COV-2) and were treated according to the following treatment groups:
Group 1: KV treatment (using OTS-3 device, Pattern A, 3 cycles) after intradermal injection volume of 100 uL with DNA dose of 100 ug, with hyaluronidase (HYA);
Group 2: NEP treatment after intradermal injection volume of 100 uL with DNA dose of 100 ug; and
Group 3: IM-EP treatment after intramuscular injection volume of 1000 uL with DNA dose of 1000 ug.

Further details regarding the test parameters for this study are shown in Table 20 below:

TABLE 20

| | | | | Total | DNA |
|---|---|---|---|---|---|
| Group Number | | | | Volume | Dose |
| (n/group) | Plasmid | Treatment | HYA | (μL) | (μg) |
| 1. (n = 7) | 9501 | KV | + | 100 | 100 |
| 2. (n = 6) | 9501 | NEP | − | 100 | 100 |
| 3. (n = 6) | 9501 | IM-EP | − | 1000 | 1000 |

The ELISA responses are shown for all Groups at Week 2 (FIG. 32A), Week 4 (FIG. 32B), and Week 5 (FIG. 32C). These results show that KV treatment with HYA outperformed the NEP and IM-EP treatment groups at all time points in terms of ELISA immunogenicity. Although the IM-EP treatments equalized with the NEP treatments as time progressed, it was not able to match the results of the KV plus HYA treatments.

T-cell responses are shown for all Groups at Week 2 (FIG. 32D) and Week 5 (FIG. 32E). These results show similar T-cell immunogenicity for IM-EP, NEP, and KV plus HYA treatments.

### Study 27

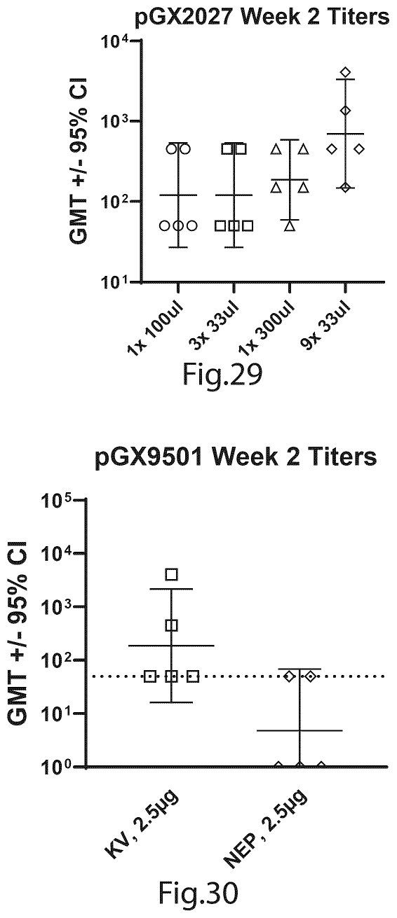

Referring now to FIG. 33, a study was conducted to compare the effects on immune responses (ELISA responses) in guinea pigs provided by different movement patterns and cycles of kinetic vacuum (KV) treatment. The KV movement patterns in this study were all performed using the same vacuum cup device, particularly the OTS-2 device found in Table 17. Subjects were injected intradermally with uniform volumes of plasmid 2303 in skin over the flank and then administered KV treatments according to four (4) movement-specific treatment groups (each Group having six (6) subjects):
Group 1: KV treatment using side-to-side (STS) movement (Pattern A, 3 cycles);
Group 2: KV treatment using a unidirectional (UNI) movement, starting off-bleb at the (−t, 0) position, employing one linear translation (one swipe) across the bleb: (−t,0)→(t,0), also referred to herein as “Pattern C” (see FIG. 7F), repeated six (6) times (i.e., 6 cycles);
Group 3: KV treatment using the same UNI movement as Group 2, but only 4 cycles thereof; and
Group 4: KV treatment using the same UNI movement as Group 2, but only 2 cycles thereof;
Details regarding the test parameters for this study are summarized in Table 21 below:

TABLE 21

| Group Number | | | | Pattern | # |
|---|---|---|---|---|---|
| (n/group) | Plasmid | Treatment | KV Movement Pattern | Acronym | Cycles |
| 1. (n = 6) | 2303 | KV | Pattern A: (0, 0)→(t, 0)→(0, 0)→(−t, 0)→(0, 0) | STS | 3 |
| 2. (n = 6) | 2303 | KV | Pattern C: (−t, 0)→(t, 0) | UNI | 6 |
| 3. (n = 6) | 2303 | KV | Pattern C: (−t, 0)→(t, 0) | UNI | 4 |
| 4. (n = 6) | 2303 | KV | Pattern C: (−t, 0)→(t, 0) | UNI | 2 |

The ELISA responses are shown for all Groups at Week 2. The results demonstrate that the unidirectional (UNI) movement patterns performed similarly with the side-to-side (STS) movement pattern. Surprisingly and unexpectedly, this held true even when reducing the number of cycles in the unidirectional (UNI) movements down to 2 cycles. These results provide evidence that the immunogenic response benefits provided by KV treatment are applicable even when employing as few as one (1) unidirectional swipe across the bleb. Moreover, when viewed in connection with the results of the Operator study discussed above (Study 18, FIG. 25), these results support a conclusion that the KV treatments disclosed herein are substantially operator-friendly, i.e., easy to perform and only require one (1) accurate swipe across the bleb.

### Study 28

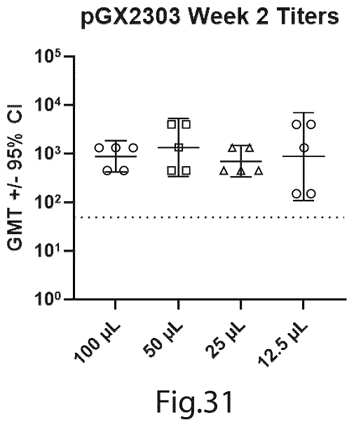

Referring now to FIGS. 34A-34B, a study compares immune responses (ELISA responses) and neutralizing activity in pigs resulting from kinetic vacuum (KV) versus needle electroporation (NEP) treatments. In this study, porcine subjects in two (2) groups (with three (3) subjects per group) were injected intradermally with uniform volumes of plasmid 9501 (which encodes the Spike glycoprotein of SARS-COV-2) and uniform DNA doses. Each subject received two (2) injections at separate locations in skin over the quadriceps, with the KV subjects (Group 2) also receiving hyaluronidase (HYA) with the injection. Following injection, Group 1 subjects were administered NEP treatment and Group 2 subjects were administered KV treatment with the OTS-2 device (see Table 0), using Pattern A, 3 cycles. For both groups, the ELISA responses at Week 2 are shown in FIG. 34A and the neutralizing titers are shown in FIG. 34B (with the dashed lines in both Figures indicating the limit of detectable titers). In terms of ELISA response (FIG. 34A), the NEP treatment responses verged on the threshold of detectability, while the KV treatments were comfortably detectable in all subjects. In terms of pseudovirus neutralization (FIG. 34B), the KV treatment group showed superior neutralizing activity over the NEP treatment group. Despite the fact that porcine subjects have been known to present challenges with demonstrating ELISA responses following intradermal injections (including with electroporation treatments), the results of this study are consistent with those showing positive ELISA responses in guinea pigs and rabbits resulting from KV treatments.

### Study 29

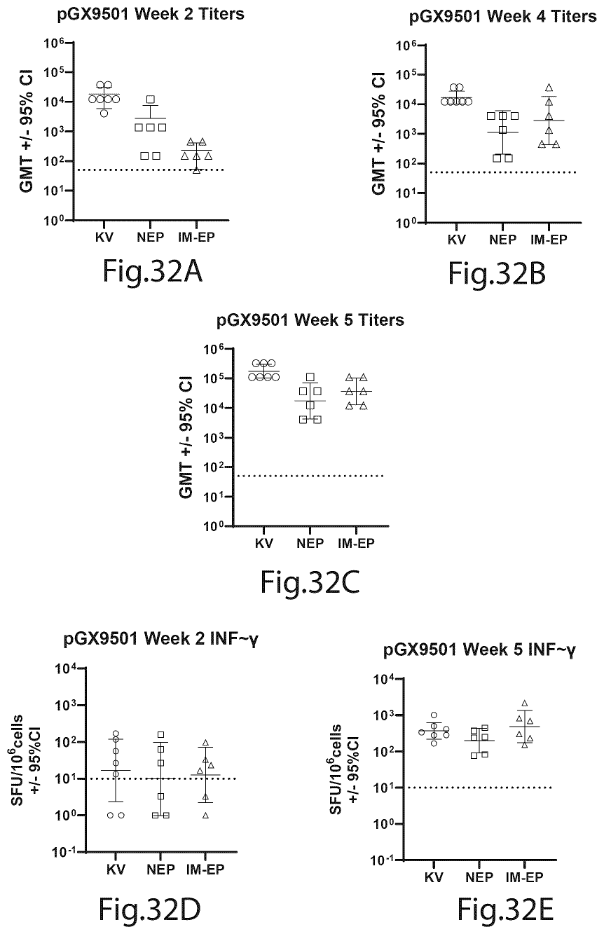

Referring now to FIGS. 35A and 35B, a study was conducted to evaluate the impacts that off-target kinetic vacuum (KV) treatments (i.e., where the vacuum cup does not contact a center of the injection bleb) has on gene (GFP) expression in guinea pigs. Subjects in four (4) Groups (with six (6) subjects per group) were injected intradermally in flank skin with a plasmid encoding the gene for GFP and then administered KV treatment employing the same movement pattern but at different offsets from the bleb center Z2 along the second direction Y perpendicular to the direction of cup translation X. The KV movements employed in this study are shown in FIG. 35A. The KV movement patterns are effectively the same as Pattern C (FIG. 7F) but at the different offset distances “d” along the second direction Y. For purposes of quantification, the offset distances d are measured along the second direction Y from the bleb center Z2 to the translation axis X3 of the vacuum cup. The KV treatments for each Group were performed using the same OTS-2 device (see Table 0), and grouped according to bleb offset distance d as follows, and as shown in FIG. 35A:
Group 1: offset distance d=0 (i.e., on-target, meaning the cup translation axis X3 intersects the bleb center Z2);
Group 2: offset distance d=t/2, wherein t is equivalent to the cup chamber diameter in this study;
Group 3: offset distance d=1, meaning the cup translation axis X3 is spaced from the bleb center Z2 by a distance equivalent to the cup chamber diameter t; and
Group 4: starting position at d=2, meaning the cup translation axis X3 is spaced from the bleb center Z2 by a distance equivalent to two (2) chamber diameters (2t).

GFP quantification for the groups is shown in FIG. 35B. The results show that the on-target KV movements (i.e., with no offset from the bleb center Z2) (Group 1) provides strong expression, with expression diminishing at an offset of d=t/2 (Group 2), and expression dropping off entirely when the offset is d=1 or greater (i.e., the vacuum cup is entirely spaced from the bleb during KV movements) (Groups 3 and 4). The results of Groups 3 and 4 are only marginally better (if at all) than GFP expression following injection only (INJ) of GFP. Notably, missing the bleb by a small distance (Group 3) performed similarly poorly to missing the bleb by a large distance (Group 4) in terms of gene expression. These results demonstrate that merely stretching the skin around the injection bleb (Group 3) is not sufficient to enhance gene expression. Instead, the results suggest that gene expression is enhanced if the injected tissue is pulled into the vacuum chamber, and increases with the percentage of injected tissue pulled into the vacuum chamber.

### Study 30

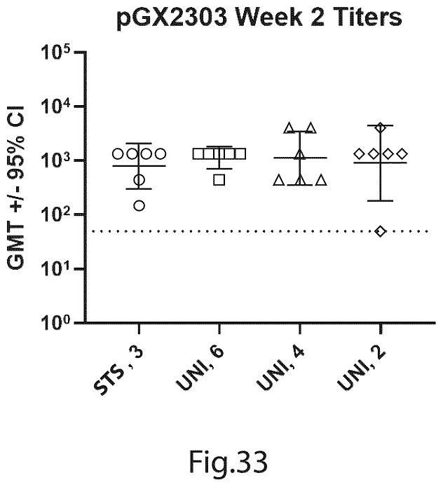

Referring now to FIG. 36A-36C, a study was conducted to compare immune responses (ELISA responses) and T-cell responses in mice following kinetic vacuum (KV) treatments with hyaluronidase (HYA) versus injection only (INJ) treatments, static vacuum (SV) treatments, and study-specific intramuscular (IM) needle electroporation (NEP) treatments, labeled below as “NEP-IM”. The subjects in this study received injections of plasmid 9517 at uniform volumes (30 uL) and uniform DNA doses (5 ug) and were treated according to the following treatment Groups (with five (5) subjects per Group):
Group 1: INJ treatment;
Group 2: KV treatment following intradermal plasmid injection with HYA in flank skin, KV treatment employed Pattern C, 4 cycles, performed by the OTS-6 device having 6 mm chamber diameter at pressure of 489 mmHg (see Table 0);
Group 3: SV treatment; and
Group 4: NEP-IM treatment after intramuscular injection, the NEP-IM was applied using three (3) needle electrodes each having 0.46 mm diameters and arranged in an isosceles triangle; four (4) consecutive pulses were applied, each having a 52 ms pulse duration, a set current of 0.2 A, a maximum voltage of 200V, and an inter-pulse delay of 250 ms.

The ELISA responses are shown for all Groups at Week 3 (FIG. 36A) and Week 4 (FIG. 36B). These results show that KV plus HYA treatments outperformed the NEP-IM treatment at all time points in terms of ELISA immunogenicity. For its part, the NEP-IM treatments showed immunogenicity at Weeks 3 and 4, though less than the KV plus HYA group. The INJ and SV treatments were not immunogenic in this study. T-cell responses are shown in FIG. 36C for all Groups at Week 4. In terms of T-cell immunogenicity, the NEP-IM treatments and KV plus HYA treatments performed similarly, which is consistent with studies in other species, and both of these treatments significantly outperformed the INJ and SV treatments. Interestingly, the NEP-IM treatments provide good T-cell responses comparable to the KV plus HYA treatments, but weaker antibody responses.

### Study 31

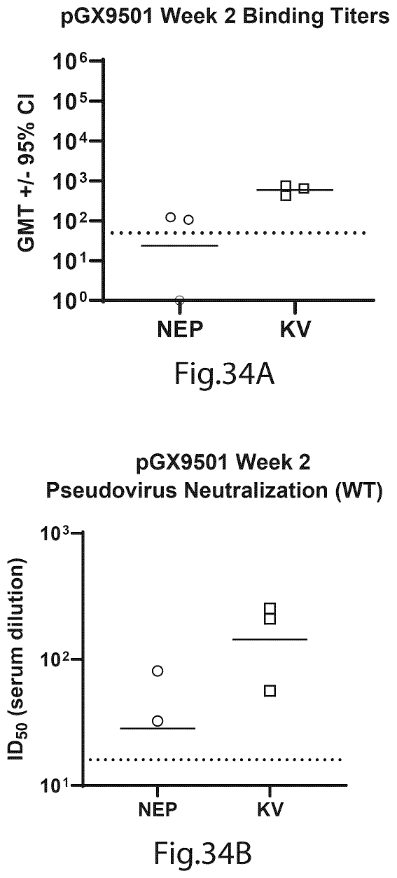

Referring now to FIG. 37, a study was conducted to evaluate the effect of hyaluronidase (HYA) dosage on kinetic vacuum (KV) treatments in terms of immune responses (ELISA responses) in guinea pigs. Subjects in five (5) groups were administered the same KV treatments following the same injections with the only difference between Groups being the HYA dosage, which was administered as follows:
Group 1: 135 U/mL;
Group 2: 75 U/mL;
Group 3: 25 U/mL;
Group 4: 10 U/mL; and
Group 5: 0 U/mL (i.e., no HYA).

Each Group involved five (5) subjects, which all received injections of plasmid 9501 in flank skin at uniform volumes (100 uL) and DNA doses (25 ug), and thereafter treated with the same KV treatment (Pattern C, 4 cycles), using the same vacuum cup, particularly the OTS-5 vacuum cup (see Table 0).

The results show that even small doses of HYA added to the plasmid injection provide significant benefits in terms of ELISA immunogenicity, even down to the 10 U/ml dose (Group 4), with a steep drop in immunogenicity from the 10 U/ml dose (Group 4) to the 0 U/ml dose (Group 5). These results demonstrate the benefit of pairing KV treatments with HYA, even at reduced dosages, such as 10% of typical HYA dosages in KV treatments discussed throughout this disclosure. Reducing HYA dosages can provide significant cost savings for KV treatments, and limits the dilutive impact of HYA on the DNA dose delivered to patients.

### Study 32

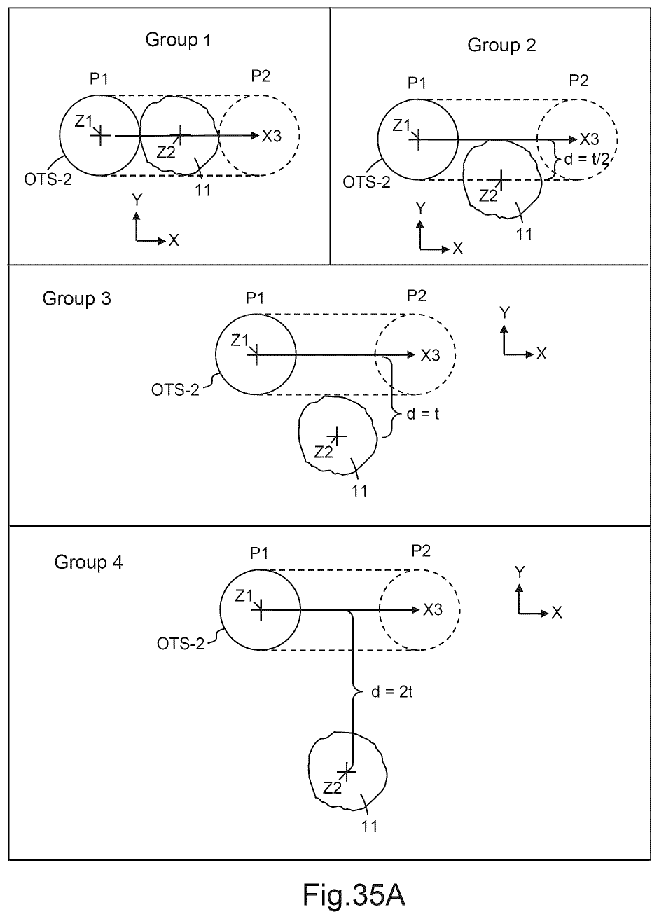

Referring now to FIG. 38, a study was conducted to compare the effects that kinetic vacuum (KV) treatments and needle electroporation (NEP) treatments have on immune cell migration in guinea pigs. Subjects in two (2) groups (with four (4) subjects per group) were injected with plasmid 5013 (which encodes the gene for GFP) at uniform volumes and DNA doses in skin over the flank. For the first group, the injections included hyaluronidase (HYA) and this group was subsequently administered KV treatment, using the OTS-5 vacuum cup (see Table 0), employing Pattern C, 4 cycles. The second group was administered NEP treatment after injection (which did not include hyaluronidase). At 3 days post-treatment, lymph nodes from both groups were analyzed to measure the number of GFP-positive cells therein to evaluate the extent of immune cell migration along the immunogenic pathway from skin, where transfection occurred, to the lymph nodes.

The results show that KV plus HYA treatments resulted in higher numbers of reporter gene-expressing cells in the lymph nodes compared to NEP treatments. These results are consistent with other studies described herein pertaining to gene expression and immune responses. Additionally, these results provide evidence that KV plus HYA treatments successfully enhance immune cell migration along immunogenic pathways (in this case, lymph node GFP trafficking) in a process downstream of the initial transfection events.

### Study 33

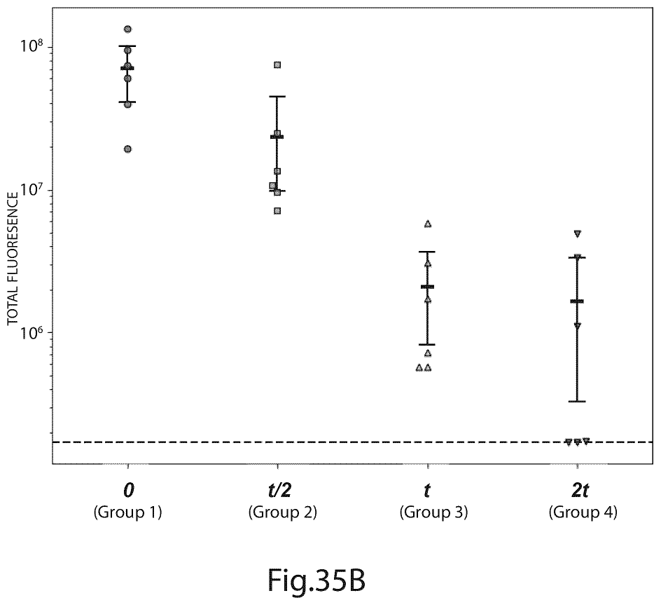

Referring now to FIG. 39, a study was conducted to evaluate the effect of hyaluronidase (HYA) in low pDNA dosage kinetic vacuum (KV) treatments in terms of immune responses (ELISA responses) in rabbits. See also Study 31 (FIG. 37). The pDNA dosage (1.5 ug) in this study was intentionally set low enough that one might expect the treatments to fail to produce a detectable ELISA response. In essence, this study evaluates whether use of HYA can “rescue” an otherwise low-success-rate KV treatment. In this study, subjects in two (2) groups (with nine (9) subjects per group) were injected with plasmid 9501 at uniform volumes and DNA doses (1.5 ug) in skin over the flank. The injections of the second group included HYA (135 U/mL), while those of the first group did not. After injection, both groups received KV treatment, using the OTS-5 vacuum cup (see Table 0), employing Pattern C, 4 cycles. The results show that even at low pDNA doses, HYA is able to improve detectable ELISA titer responses, with most subjects in the second group producing detectable titer levels, while only two (2) subjects in the first group (no HYA) having detectable levels. These results show trends that adding HYA to KV treatments can enhance or rescue immunogenicity conditions that may otherwise be poorly or non-immunogenic.

### Study 34

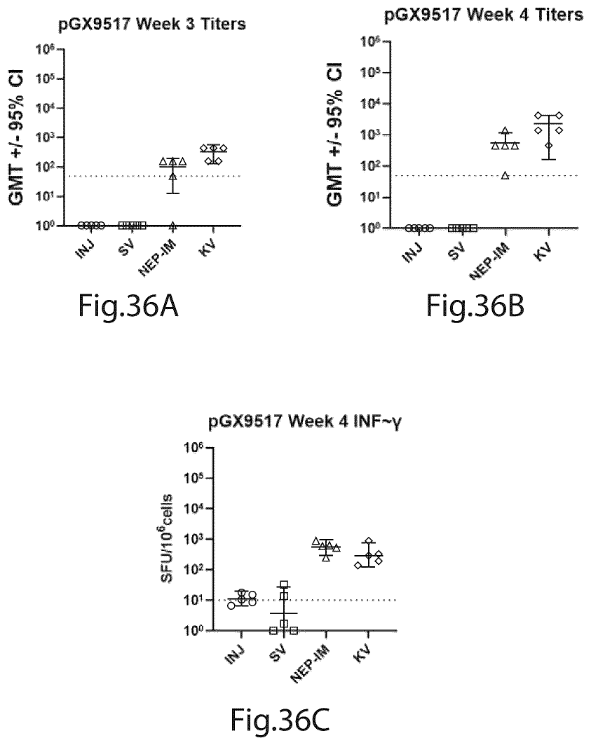

Referring now to FIG. 40, a study similar to Study 33 (FIG. 39) was conducted in rabbits but at a medium pDNA dosage, particularly at 15 ug (with is ten times (10×) the pDNA dosage used in Study 33). In the present study, a different DNA plasmid was used: plasmid 2303. All other conditions (beside pDNA plasmid and dosage) in this study were the same as in Study 33. Again, subjects in two (with nine (9) subjects per group) were injected with plasmid 2303 at uniform volumes and “medium” DNA doses (15 ug) in skin over the flank. The injections of the second group included hyaluronidase (HYA) at 135 U/mL, while those of the first group did not. After injection, both groups received KV treatment, using the OTS-5 vacuum cup (see Table 0), employing Pattern C, 4 cycles. The results are consistent with those of Study 33, showing here that at medium pDNA doses, HYA improves ELISA titer responses in KV treatments.

When combined, the results of Study 33 (FIG. 39) and Study 34 (FIG. 40) suggest that adding HYA to KV treatments not only improves immunogenicity (and may be seen as dose-sparing), but does so to the extent that KV treatments involving HYA may improve treatment outcomes for subjects that would otherwise be unlikely to achieve meaningful immune responses.

### Study 35

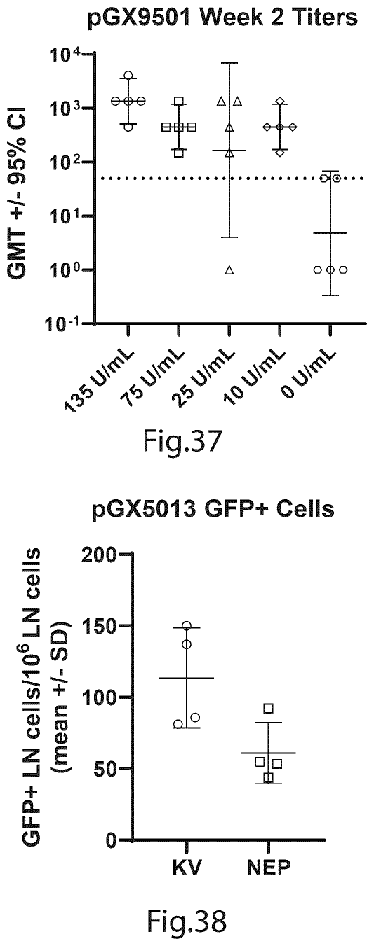

Referring now to FIG. 41, another study was conducted to evaluate the effects of kinetic vacuum (KV) treatments on immune cell migration, this time in rabbits. In this regard, this study has similarities to Study 32 (FIG. 38), which was conducted in guinea pigs. In the present study, subjects in four (4) groups (with six (6) subjects per group) were injected with plasmid 5013 (which encodes the gene for GFP) at uniform volumes and DNA doses in skin over the flank. After injection, each group received KV treatment, using the OTS-5 vacuum cup, employing Pattern C, 4 cycles. The lymph nodes were analyzed at different days post-treatment to measure the number of GFP-positive cells therein for each group as follows: Group 1 at Day 1; Group 2 at Day 2; Group 3 at Day 3; and Group 4 at Day 7. This analysis aims to identify the timing of immune cell migration from the skin to the lymph nodes.

The results show strong immune cell migration occurring at Days 1 and 2, with a drop-off at Day 3, and then migration remaining level to Day 7. These results suggest that the majority of the immune cell migration (in this case, lymph node GFP trafficking) occurs within the first 48 hours. These results, along with those of Study 32 (FIG. 38), provide evidence that the immunogenic response involves antigen trafficking along immunogenic pathways (e.g., lymph node trafficking) concentrated in the first 2 days following transfection.

### Study 36

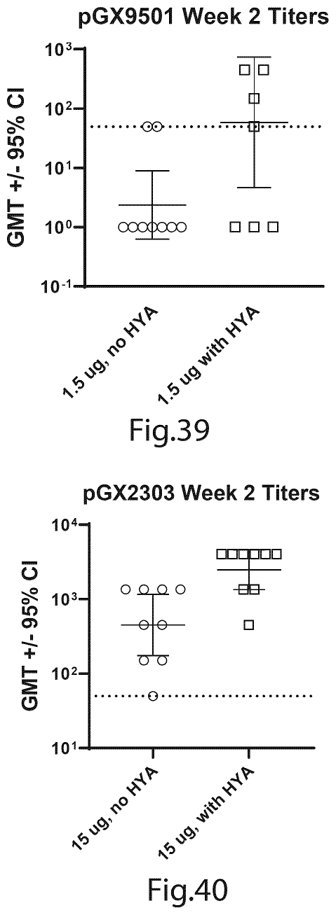

Referring now to FIG. 42, a study was conducted to evaluate the “tradeoff” relationship between DNA dosage and hyaluronidase (HYA) dosage in a given total injection volume for kinetic vacuum (KV) treatments in guinea pigs. Subjects in four (4) groups (with five (5) subjects per group) were administered the same KV treatments following injections of plasmid 2303 of various percentages of DNA dose to HYA dose. For Groups 1-3, the DNA and HYA doses were constrained within a uniform total injection volume (thus, the HYA dosage was made at the sacrifice of DNA dosage, and vice versa). For Group 4, the total injection volume was unconstrained and involved a high dose for both DNA and HYA dosage. The dosages in Groups 1-4 were as follows:
Group 1: DNA dose of 5 ug; HYA dose of 75 U/mL (i.e., a 50/50 mix, that is, a half dose of DNA and a half dose of HYA);
Group 2: DNA dose of 9 ug; HYA dose of 12.5 U/mL (i.e., a DNA dose diluted by 10% by the presence of a low dose of HYA); and
Group 3: DNA dose of 10 ug; HYA dose of 0 U/mL (i.e., 100% DNA dosage, no HYA);
Group 4: DNA dose of 10 ug; HYA dose of 135 U/mL (i.e., high HYA dosage with no sacrifice to DNA dosage).

Details regarding the test parameters for this study are summarized in Table 22 below:

TABLE 22

| Group Number | | | DNA Dose | HYA dose |
|---|---|---|---|---|
| (n/group) | Plasmid | Treatment | (ug) | (U/mL) |
| 1. (n = 5) | 2303 | KV | 5 | 75 |
| 2. (n = 5) | 2303 | KV | 9 | 12.5 |
| 3. (n = 5) | 2303 | KV | 10 | 0 |
| 4. (n = 5) | 2303 | KV | 10 | 135 |

For all Groups, the injections were performed intradermally in skin over the flank. All KV treatments in this study were performed using the OTS-5 vacuum cup (see Table 0), employing Pattern C, 4 cycles.

The results demonstrate that the presence of HYA at each of the dosages tested (Groups 1, 2 and 4) enhanced the ELISA titer immune response. Comparatively, the no-HYA subjects (Group 3) barely produced any measurable ELISA response, if at all. Interestingly, the high-DNA, high-HYA formulation (Group 4) performed similar to the 50/50 formulation (Group 1), wherein both Groups 1 and 4 demonstrated measurable ELISA responses, with Group 2 (the 90% DNA, low-HYA formulation) outperforming both of Groups 1 and 4. These results suggest that DNA dosage need not be sacrificed for increased HYA dosage in order to produce enhanced immunogenicity, so long as some HYA is added. Moreover, these results demonstrate that a low-HYA dosage (Group 2) can outperform a high-HYA dosage (Group 4), interestingly, even a high-HYA dosage with no sacrifice to DNA dosage (again Group 4). Another interesting observation from these results is that adding HYA to plasmid injections produced beneficial immunogenicity, even if DNA dosage is sacrificed in the process (e.g., compare Group 1 (50/50 mix) to Group 3 (100% DNA dose, no HYA). With consideration towards potential clinical use of KV+HYA delivery, these results support the inclusion of HYA at low doses (such as, but not limited to, 10% of the total injection volume) in the plasmid injections for KV treatments. Overall, these results provide exciting implications regarding the potential for significant cost-savings to be gained from reducing HYA to even a small fraction of historical dosages in plasmid injections for KV treatments without sacrificing its beneficial effects.

### Study 37

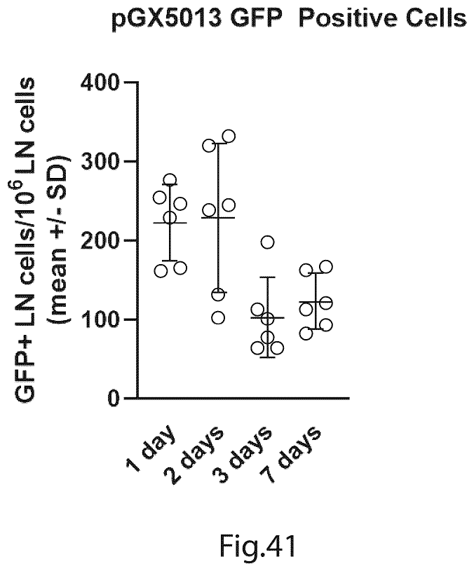

Referring now to FIG. 43, a study was conducted to compare the effect on ELISA immune response provided by adjusting the number of kinetic vacuum (KV) translational movements (cycles, in this case “swipes”) on otherwise similar KV treatments. Subjects in four groups (with five (5) subjects per group) were intradermally injected with plasmid 2027 at uniform volumes and DNA dosages in skin over the flank, and subsequently received KV treatments by the same OTS vacuum cup, particularly the OTS-5 vacuum cup (see Table 0), employing different numbers of cycles of the same KV movement pattern, particularly Pattern C across the injection bleb (see FIG. 7F). The cycles administered to each group were as follows:
Group 1 received one (1) cycle (i.e., one swipe);
Group 2 received two (2) cycles;
Group 3 received three (3) cycles; and
Group 4 received four (4) cycles.

The Results show substantially equivalent immune responses in terms of ELISA responses regardless of the number of swipes employed across the injection bleb. Additionally, when viewed in connection with the results of the above-described Operator study (Study 18, FIG. 25), the unidirectional (UNI) movement study (Study 27, FIGS. 33A and 33B), and the off-target KV study (Study 29, FIGS. 35A and 35B), these results support a conclusion that the KV treatments disclosed herein are substantially operator-friendly, i.e., easy to perform and only require one (1) accurate swipe across the injection bleb to achieve a beneficial immune response. Stated in simpler terms, these studies provide evidence that one swipe across the bleb is as effective as multiple swipes across the bleb in order to administer an immunogenic KV treatment.

### Study 38

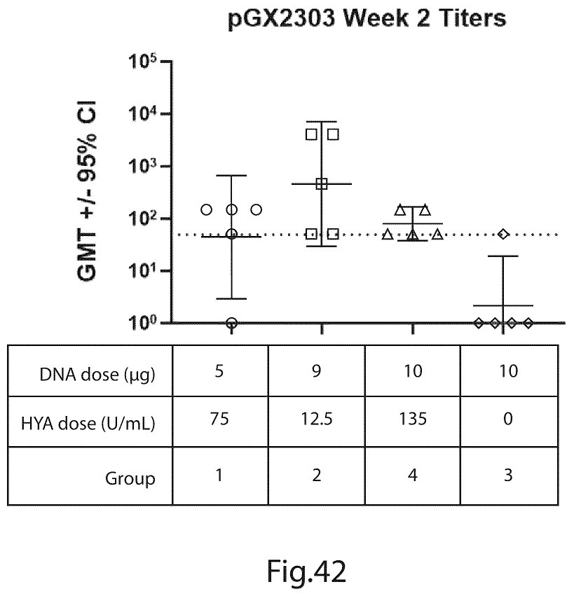

Referring now to FIG. 44, a study was conducted to explore the effects provided by kinetic vacuum (KV) treatments on reduced dosages of mRNA-1273 (an mRNA-based vaccine for SARS-COV-2) on immune responses (binding ELISA responses) in guinea pigs. In this study, subjects in four (4) groups (with five (5) subjects per group) received injections of mRNA-1273 at either a low mRNA dose (0.1 ug) or a high mRNA dose (1 ug). Additionally, for each dosage in this study, the mRNA-1273 plasmid was injected either via intramuscular (IM) injection (which is Moderna's protocol injection) or via intradermal (ID) injection, with the intradermal injections follows by KV treatments using the same vacuum cup (the OTS-5 vacuum cup, see Table 0) employing the same movement pattern (Pattern C), 4 cycles. The dosages in Groups 1-4 were as follows:
Group 1: mRNA dose of 1 ug (“high dose”, for purposes of this study), via intramuscular (IM) injection;
Group 2: mRNA dose of 0.1 ug (“low dose”, for purposes of this study), via intramuscular (IM) injection;
Group 3: mRNA dose of 1 ug (high dose), via intradermal (ID) injection followed by KV treatment; and
Group 4: mRNA dose of 0.1 ug (low dose), via intradermal (ID) injection followed by KV treatment.

Details regarding the test parameters for this study are summarized in Table 23 below:

TABLE 23

| Group Number | | Injection | Additional | mRNA Dose |
|---|---|---|---|---|
| (n/group) | Agent | Type | Treatment | (ug) |
| 1. (n = 5) | mRNA-1273 | IM | — | 1 |
| 2. (n = 5) | mRNA-1273 | IM | — | 0.1 |
| 3. (n = 5) | mRNA-1273 | ID | KV | 1 |
| 4. (n = 5) | mRNA-1273 | ID | KV | 0.1 |

The ELISA responses at Week 2 are shown for all Groups (with the dashed line indicating the limit of detectable titers). The results show that, at the high dose level (1 ug), both IM delivery (Group 1) and ID delivery plus KV treatment (Group 3) produced similar immunogenicity in terms of ELISA titers. At the low dose level (0.1 ug), the IM delivery (Group 1) had entirely undetectable ELISA responses, while the ID delivery plus KV treatment (Group 4) remained immunogenic, although experiencing a drop from its high-dose counterpart (Group 3). These results demonstrate that at low mRNA doses, delivering mRNA-1273 intradermally (ID) with supplemental KV treatment is far superior to intramuscular (IM) delivery in terms of ELISA response.

### Study 39

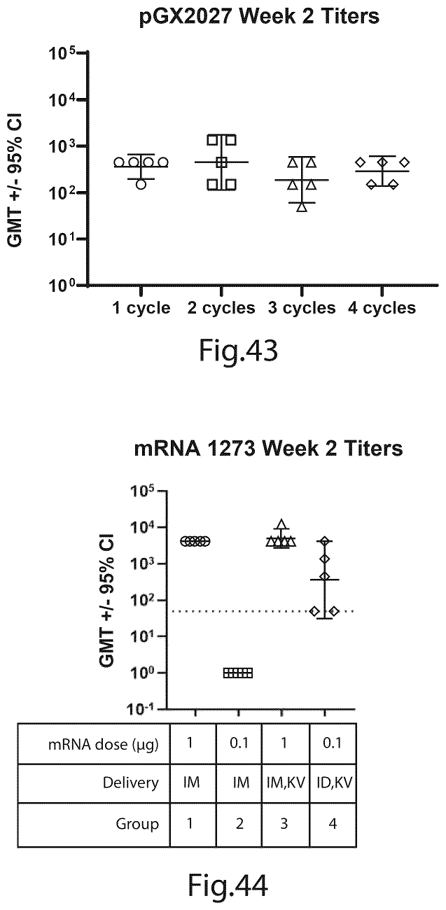

Referring now to FIG. 45, a follow-up study to Study 38 (FIG. 44) was conducted to further explore the effects provided by kinetic vacuum (KV) treatments on reduced dosages of mRNA-1273 on immune responses (binding ELISA responses) in guinea pigs. In this follow-up study, KV treatments following intradermal (ID) injections of mRNA-1273 at, for purposes of this study, “high”, “medium”, and “low” doses (1 ug, 0.5 ug, and 0.1 ug, respectively) were compared to high- and low-dose intradermal (ID) injection-only (INJ) treatments of mRNA-1273 (as opposed to intramuscular (IM) injection-only treatments of the mRNA, as in Study 38). Thus, in the present study, all injections were intradermal (ID). The KV treatments were administered using the same vacuum cup and same movement pattern and cycles as in Study 38, i.e., the OTS-5 vacuum cup (see Table 0), using Pattern C, 4 cycles. The dosages in Groups 1-5 were as follows:
Group 1: mRNA dose of 1 ug (high dose);
Group 2: mRNA dose of 0.1 ug (low dose);
Group 3: mRNA dose of 1 ug (high dose), followed by KV treatment;
Group 4: mRNA dose of 0.1 ug (low dose), followed by KV treatment; and
Group 5: mRNA dose of 0.5 ug (medium dose), followed by KV treatment.

Details regarding the test parameters for this study are summarized in Table 24 below:

TABLE 24

| Group Number | | Injection | Additional | mRNA Dose |
|---|---|---|---|---|
| (n/group) | Agent | Type | Treatment | (ug) |
| 1. (n = 5) | mRNA-1273 | ID | — | 1 |
| 2. (n = 5) | mRNA-1273 | ID | — | 0.1 |
| 3. (n = 5) | mRNA-1273 | ID | KV | 1 |
| 4. (n = 5) | mRNA-1273 | ID | KV | 0.1 |
| 5. (n = 5) | mRNA-1273 | ID | KV | 0.5 |

The ELISA responses at Week 2 are shown for all Groups (with the dashed line indicating the limit of detectable titers). The results show that, at both the high dose (1 ug) and low dose (0.1 ug) mRNA ID injections, the KV treatments (Groups 3 and 4) outperformed the ID injection-only treatments (Groups 1 and 2) head to head in terms of ELISA titers, although the high mRNA dose ID injections (Group 1) were more immunogenic than the low mRNA dose KV treatments (Group 4). The medium mRNA dose (0.5 ug) KV treatments (Group 5) modestly outperformed the high mRNA dose ID injection-only treatments (Group 1). Interestingly, at low mRNA dose levels (0.1 ug), the ID injection-only treatments (Group 2) were more immunogenic than the intramuscular (IM) injection-only treatments from Study 38 (Group 2 therein).

When the results of the present study are combined with those of Study 38, the data demonstrates that at low mRNA doses, delivering mRNA-1273 intradermally (ID) with supplemental KV treatment is far superior to both intramuscular (IM) and intradermal (ID) injection-only treatments in terms of ELISA response. The combined results also show that the medium mRNA dose KV treatments provided comparable immunogenicity to both ID and IM injection-only treatments of high-dose mRNA-1273. Thus, the combined results suggest that injecting mRNA-1273 intradermally followed by KV treatment can provide dose-sparing effects for mRNA-1273 injections, whether injected intramuscularly (IM) or intradermally (ID), without sacrificing immunogenicity. Additionally, the delivery of lipid nanoparticle mRNA into skin using KV was shown to be more immunogenic than delivery into muscle, or into skin without using KV, suggesting that KV generally enhances cellular uptake of foreign cargo, not only plasmid DNA

### Study 40

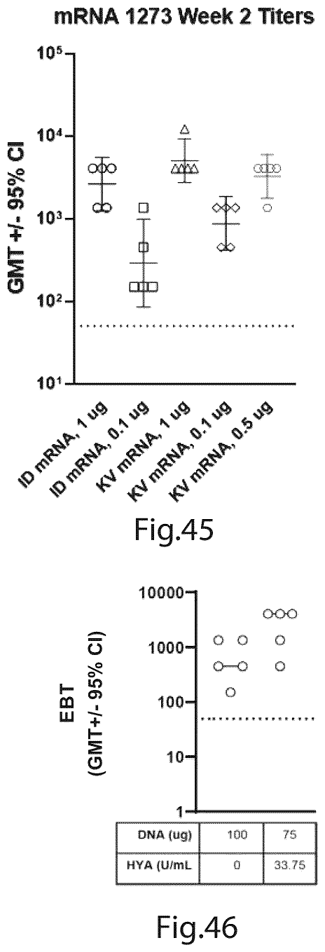

Referring now to FIG. 46, another hyaluronidase dosing study was conducted, particularly to evaluate the “tradeoff” relationship between DNA dosage and hyaluronidase (HYA) dosage in high-DNA dosage scenarios for kinetic vacuum (KV) treatments in guinea pigs. Subjects in two (2) groups (with five (5) subjects per group) were administered the same KV treatments following injections of plasmid 2303 at high doses, as follows:
Group 1: DNA dose of 100 ug; HYA dose of 0 U/mL (i.e., 100% DNA dosage, no HYA); and
Group 2: DNA dose of 75 ug; HYA dose of 33.75 U/mL (i.e., a DNA dose diluted 25% by the presence of HYA).

The results show that at high DNA doses, adding HYA improved immunogenicity, even at the expense of reducing the DNA dosage by 25%.

### Observations

Based on the data and results from the studies described above with reference to FIGS. 8-46, the inventors had made the following observations. Kinetic vacuum treatments provide comparable immune responses to vacuum electroporation (VEP) or needle electroporation (NEP) across multiple studies, and greater responses in guinea pigs or rabbits in multiple studies (see FIGS. 17-18, 23A-23B, 30, 32A-32E, 34A-34B). Additionally, the kinetic vacuum treatments improved immune responses compared to the static vacuum treatments in all studies herein, in both rabbits and guinea pigs. Moreover, kinetic vacuum treatments provided superior gene expression relative to vacuum electroporation (VEP) treatments and, in the study shown in FIG. 8, kinetic vacuum treatments produced a larger transfection zone. Although kinetic vacuum treatments were observed to cause acute redness at the treatment site, it vanished rapidly and did not cause any scabbing, burning, or other tissue damage, unlike the electroporation treatments in the studies herein that caused more tissue damage and required longer healing times to recover to normal compared to the kinetic vacuum treatments. KV also led to superior antigen trafficking into the lymph nodes than NEP (FIG. 38), and was shown to be dose-sparing, generating stronger immune responses compared to NEP when suboptimal (poorly or non-immunogenic) DNA vaccine doses were used.

Furthermore, for kinetic vacuum treatments, six (6) swipes (three back-and-forth cycles) outperformed a half swipe, though even a single swipe was subsequently shown to be similarly immunogenic to patterns of multiple swipes. Furthermore, the magnitude of gene expression was associated with the percentage of the injection site contacted by the swipe; missing a percentage of the injection site correspondingly reduced the gene expression. It was also observed that the addition of hyaluronidase (HYA) significantly improved both gene expression and immune responses for kinetic vacuum treatments, but did not similarly benefit static vacuum or electroporation treatments. HYA concentrations ranging from 12.5 U/mL to 135 U/mL were all similarly effective in enhancing immunogenicity of KV delivery of DNA vaccines, suggesting that even a small amount of HYA can trigger this benefit. The magnitude of the immune response following KV delivery increased with increasing vacuum strength, and with increasing cup diameter, until cup diameter was roughly similar to the diameter of the injection site in skin. These studies also demonstrated that decreasing the injection volume in kinetic vacuum treatments may slightly reduce immunogenicity. It was also observed that supplementing kinetic vacuum treatments with vacuum electroporation (VEP) did not produce beneficial results over kinetic vacuum treatments. Furthermore, kinetic vacuum treatments that include hyaluronidase were observed to approach parity with lipid nanoparticle mRNA-level immune responses, with superior cellular responses, although DNA-based kinetic vacuum treatments still required about ten times (10×) the dose of mRNA to achieve near parity. The use of a DNA-launched nanoparticle plasmid, rather than a plasmid encoding monomeric antigen, delivered using KV+HYA, was capable of eliciting more durable immune responses than lipid nanoparticle mRNA using the same dose of nucleic acid in each group. Lastly, the delivery of lipid nanoparticle mRNA into skin using KV was shown to be more immunogenic than delivery into muscle, or into skin without using KV, suggesting that KV generally enhances cellular uptake of foreign cargo, not only plasmid DNA.

### Modifications

It should be appreciated that the various parameters of the vacuum cups, systems, and kinetic vacuum treatment techniques described above are provided as exemplary features for providing vacuum-enhanced transfection of tissue. These parameters can be adjusted as needed without departing from the scope of the present disclosure. For example, although the kinetic vacuum treatments and vacuum cups described above are mainly described in relation to enhancing transfection of skin tissue, the kinetic vacuum treatments and vacuum cups herein can be adapted for treating adipose tissue and/or muscle tissue for enhancing transfection therein.

It should also be appreciated that in additional embodiments, the various vacuum cups 2, 102, 302, 402 and supporting components (see FIGS. 1A, 3A-3B, and 5A-5B) can be provided in a kit that includes a plurality of vacuum cups 2, 102, 302, 402 that a user can employ interchangeably.

Although the disclosure has been described in detail, it should be understood that various changes, substitutions, and alterations can be made herein without departing from the spirit and scope of the invention as defined by the appended claims. Moreover, the scope of the present disclosure is not intended to be limited to the particular embodiments described in the specification. In particular, one or more of the features from the foregoing embodiments can be employed in other embodiments herein. As one of ordinary skill in the art will readily appreciate from that processes, machines, manufacture, composition of matter, means, methods, or steps, presently existing or later to be developed that perform substantially the same function or achieve substantially the same result as the corresponding embodiments described herein may be utilized according to the present disclosure.

What is claimed:

1. A method of enhancing delivery of an agent into tissue, comprising:
placing a housing that defines a chamber adjacent to a surface of the tissue, wherein the placing step locates the chamber adjacent to an injection site at which the agent was injected into the tissue;
applying vacuum pressure to the chamber, thereby drawing a portion of the tissue through an opening of the chamber and into the chamber; and
moving the housing relative to the tissue while the vacuum pressure is applied for enhancing agent delivery in the tissue.

2. The method of claim 1, wherein the moving step comprises translating the housing along a path of travel that causes at least a portion of the chamber to traverse a center of the injection site.

3. The method of claim 1, wherein the placing step locates the chamber at a first location that is spaced from a center of the injection site at a first offset distance measured from the center of the injection site along a direction, the translating step comprises translating the housing from the first location and across the center of the injection site along the direction to a second location, wherein the second location is spaced from the center of the injection site at a second offset distance measured along the direction.

4. The method of claim 3, wherein at least one of the first and second offset distances is no less than a maximum interior dimension of the chamber measured along the direction.

5. The method of claim 4, wherein the first and second offset distances are each no less than the maximum interior dimension of the chamber.

6. The method of claim 3, wherein the step of translating the housing from the first location to the second location is performed at least twice.

7. The method of claim 2, wherein the translating step comprises a first step of translating the housing along a straight path at least partially across the injection site in a first direction, and subsequently a second step of translating the housing along a straight path at least partially across the injection site in a second direction opposite the first direction.

8. The method of claim 3, wherein the first and second translating steps are repeated a plurality of times.

9. The method of claim 1, wherein the moving step comprises at least one of:
translating the housing along a substantially circular path around the injection site, such that at least a portion of the chamber overlies a center of the injection site while the housing translates along at least a portion of the substantially circular path; and
pivoting the housing about a central axis of the vacuum chamber, the central axis oriented substantially orthogonal to the surface of the tissue over which the chamber is placed.

10. The method of claim 1, wherein the applied vacuum pressure is in a range from about −300 mmHg to about −760 mmHg during the moving step.

11. The method of claim 11, wherein the applied vacuum pressure is in a range from about −400 mmHg to about −600 mmHg during the moving step

12. The method of claim 1, wherein the portion of the tissue drawn into the chamber is drawn into contact with at least one protrusion of the housing located within the chamber, thereby deforming the tissue that contacts the protrusion.

13. A method of enhancing delivery of an agent into tissue, comprising:
injecting an agent into tissue of the subject, thereby defining an injection site at a surface of the tissue;
placing a housing that defines a chamber at or adjacent the injection site;
applying vacuum pressure to the chamber, thereby drawing a portion of the tissue through an opening of the chamber and into the chamber; and
moving the housing relative to the tissue while the vacuum pressure is applied for enhancing agent delivery in the tissue.

14. The method of claim 13, wherein the moving step comprises translating the housing along a path of travel that causes at least a portion of the chamber to traverse a center of the injection site one or more times.

15. The method of claim 13, wherein the applied vacuum pressure is in a range from about −300 mmHg to about −760 mmHg during the moving step.

16. The method of claim 13, wherein the tissue is skin tissue, the surface of the tissue is a skin surface, and the injecting step comprises performing a Mantoux injection in the skin tissue.

17. The method of claim 16, wherein the injected agent includes a spreading agent.

18. The method of claim 17, wherein the spreading agent is hyaluronidase.

19. The method of claim 18, wherein the hyaluronidase comprises from about 5% to about 20% of a total injection volume of the agent.

20. The method of claim 13, wherein the injected agent comprises a plasmid containing mRNA and lipid nanoparticles.

21. The method of claim 13, wherein the injected agent comprises a plasmid containing DNA-launched nanoparticles.

22. The method of claim 13, wherein:
the injecting step comprises injecting a total volume of the agent into the tissue, wherein the total volume is partitioned into a plurality of sub-volumes, such that the injecting step comprises performing a plurality of injections into the tissue at a plurality of respective injection sites, each of the plurality of injections injecting a respective one of the plurality of sub-volumes into the tissue; and
the moving step comprises translating the housing along a path of travel that causes the chamber to traverse at least a majority of the plurality of injection sites.

23. A system for vacuum-enhanced agent delivery into tissue in vivo, comprising:
a housing defining a chamber and an opening into the chamber;
at least one port extending through the housing, wherein the at least one port is remote from the at least one opening and is connectable to a vacuum source, such that the at least one port is configured to communicate vacuum pressure from the vacuum source to the chamber,
wherein the housing is configured to communicate a vacuum field to a portion of the tissue and thereby draw the portion of tissue through the opening and at least momentarily hold the portion of the tissue in the chamber, and the housing is further configured to move relative to the tissue while the vacuum field is communicated to the tissue, wherein the relative motion deforms at least some of the tissue; and
one or more features disposable between a distal end of the housing and a surface of the tissue for reducing sliding friction between the housing and the tissue.

24. The system of claim 23, wherein the one or more features is selected from the group comprising:
a lubricant; and
rollers coupled to the distal end of the housing.

25. The system of claim 23, further comprising a handle member, wherein the housing is attachable to the handle member.

26. The system of claim 23, wherein the housing is configured to communicate the vacuum field at a vacuum pressure in a range from about −300 mmHg to about −760 mmHg during movement of the housing relative to the tissue.

27. The system of claim 26, wherein the chamber has a circular cross sectional shape in a plane orthogonal to a central axis of the chamber, and an interior surface of the housing within the chamber defines a chamber diameter in a range from about 5 mm to about 15 mm.
